(19) United States
(12) Patent Application Publication
(10) Pub. No.: US 2026/0265408 A1
(43) Pub. Date: Sep. 10, 2026

(54) GD2-SPECIFIC CHIMERIC ANTIGEN RECEPTOR EFFECTOR CELLS FOR USE IN THE TREATMENT OF SOLID TUMORS, POSSIBLY IN COMBINATION WITH ENHANCER OF ZESTE HOMOLOG 2 (EZH2) INHIBITORS

(71) Applicant: OSPEDALE PEDIATRICO BAMBINO GESU', Rome (IT)

(72) Inventors: Franco LOCATELLI, Rome (IT); Concetta QUINTARELLI, Rome (IT); Biagio DE ANGELIS, Rome (IT)

(21) Appl. No.: 19/489,204

(22) PCT Filed: May 31, 2024
(86) PCT No.: PCT/IT2024/050116

§ 371 Date: Dec. 1, 2025

(30) Foreign Application Priority Data
Jun. 1, 2023 (IT) 102023000011250

(51) Int. Cl. C07K 16/30; A61K 40/11; A61K 40/31; A61K 40/42; A61P 35/00; C07K 14/705; C12N 5/0783

(52) U.S. Cl. CPC C07K 16/3084; A61K 40/11; A61K 40/31; A61K 40/4258; A61P 35/00; C07K 14/7051; C07K 14/70517; C07K 14/70521; C07K 14/70578; C12N 5/0636; C07K 2317/622; C12N 2510/00

(57) ABSTRACT

GD2-specific Chimeric Antigen Receptor effector cells for use in the treatment of solid tumors, possibly in combination with Enhancer of Zeste Homolog 2 (EZH2) inhibitors. —The present invention concerns a GD2 Specific Chimeric Antigen Receptor (GD2.CAR) effector cells for the treatment of solid tumours, in particular both extracranial GD2+ tumours (as soft and bone sarcomas, neuroblastoma, melanoma, lung cancer, bladder cancer and retinoblastoma) and brain tumours. Moreover, the present invention concerns also the use of GD2.CAR genetically modified effector cells in combination with Enhancer of Zeste Homolog 2 (EZH2) inhibitors for use in the treatment of solid tumors.

The present invention concerns a GD2 Specific Chimeric Antigen Receptor (GD2.CAR) effector cells (means T cells, or innate cells: NK; NK-T cells, etc.) for the treatment of solid tumours, in particular both extracranial GD2+ tumours (as soft and bone sarcomas, neuroblastoma, melanoma, lung cancers, breast cancer, bladder cancer and retinoblastoma) and brain tumours. In particular, the present invention concerns a vector including the cassette coding for GD2.CAR gene, a method for the production thereof and GD2.CAR genetically modified effector cells (such as T cells or innate cells such as NK and NK-T cells) for the treatment of GD2+ solid tumours including brain tumours such as Medulloblastoma and Glioma tumor cells. Moreover, the present invention concerns also the use of GD2.CAR genetically modified effector cells in combination with Enhancer of Zeste Homolog 2 (EZH2) inhibitors for use in the treatment of solid tumors.

Gangliosides are a subfamily of glycosphingolipids that contain one or more sialic acid residues, consisting of a hydrophilic sialic acid-containing oligosaccharide chain and a hydrophobic neuraminide. Gangliosides can be divided into monosialogangliosides (GM), disialogangliosides (GD), trisialogangliosides (GT) and tetrasialoganglioside (GQ) according to the number of sialic acid residues per molecule¹. Disialoganglioside with two glycosyl groups (GD2) are markedly increased in pathological conditions such as cancers and neurodegenerative diseases. This glycosphingolipid involved in signal transduction, cell-cell recognition, and tumor cell metastasis.

While most normal tissues express very low levels of GD2, on the contrary many tumours are characterized by its very high expression². In particular, GD2 is found highly expressed in melanoma^{3, 4}, in sarcoma⁵, breast cancers⁶, bladder cancers², neuroblastoma⁷, retinoblastoma⁸, small cell lung cancers⁹, gliomas¹⁰. Of interest, H3K27M-mutant diffuse intrinsic pontine glioma (H3K27M-mutant DIPG) primary tumor cells showed the highest expression of GD211.

Sarcomas are rare mesenchymal tumors that include a variety of bone and soft-tissue tumors that affect all ages but are relatively more common in the paediatric age group, accounting for about 10-15% of childhood cancers¹². In children and adolescents, the most common sarcomas include Osteosarcoma (OS), Rhabdomyosarcoma (RMS), Ewing's sarcoma (EWS), and desmoplastic small round cell tumors (DSRCT), while other sarcomas, such as leiomyosarcomas and liposarcomas, typically present in older patients¹³. Despite their rarity, they constitute significant mortality burden of about 13% of cancer related deaths in patients 0-19 years of age¹⁴. Moreover, in spite of increasing efforts focusing on identification of targeted therapies, including tyrosine kinase inhibitors for soft tissue and bone sarcomas, clinical results have not significantly improved for these patients. Thus, novel treatment strategies to improve sarcoma patient outcomes are urgently needed¹⁵.

Ewing sarcoma (EwS) is an aggressive solid mesenchymal malignancy arising in bone and soft tissues¹⁶. EwS is characterized by a specific chromosomal translocation, most commonly of chromosomes 22 and 11 (t (11,22) (q24; 12)), resulting in the aberrant chimeric transcription factor EWSR1-FLI1¹⁷. Enhancer of Zeste Homolog 2 (EZH2) inhibitor Tazemetostat (E7438/EPZ6438)¹⁸ has been reported to up-regulate GD2 expression in Ewing sarcoma¹⁹. In particular, EZH2 acts as a histone methyltransferase, and it silences genes involved in cell differentiation in a highly context-dependent manner, by depositing repressive histone marks at histone 3 lysine 27 (H3K27me3). High-level EZH2 expression is induced in EwS cell lines as a direct consequence of EWSR1-FLI120. EZH2 modulates GD2 Expression in EwS Cells by regulating the expression of genes involved in GD2 biosynthesis. Pharmacological inhibition of EZH2 selectively upregulates surface GD2 expression in EwS cells¹⁹. Regarding the inhibition of EZH2, Wiebel M. et al. reported that the use of the inhibition of the histone methyltransferase Enhancer of Zeste Homolog 2 (EZH2) failed to increase GD2 expression or in OS cell lines²¹.

Rhabdomyosarcoma (RMS) is a family of soft tissue sarcoma of childhood and adolescence that includes Fusion Positive FP-RMS, harboring the fusion oncoprotein PAX3/7-FOXO1 and FN-RMS, often mutant in the RAS pathway.

Brain tumors are the most common solid cancers of children²². Standard therapy for these diseases includes surgical resection, radiation, and, in selected cases, chemotherapy. Despite aggressive treatment, many patients have a poor long-term outcome or, frequently, develop treatment-related long-term sequelae, including hormone dysfunction, sensory-motor and neurocognitive impairments²³.

Pediatric low-grade gliomas (pLGGs) are the most common pediatric brain tumor representing between 40 and 50% of all pediatric CNS tumors²⁴. The World Health Organization (WHO) classifies these as grade I and grade II tumors²⁵, with pilocytic astrocytoma being the most common subtype. Although these patients have a good overall survival²⁶, they often have poor progression-free survival (PFS)²⁷.

Pediatric high-grade gliomas (pHGGs) represent approximately 10-25% of all pediatric CNS tumors²⁸. The WHO classifies these as grade III and IV tumors, and although histologically they often appear identical to their adult counterparts, they are clinically and molecularly distinct²⁹. The survival outcomes for these patients are in stark contrast to pLGG as most patients will succumb to their disease within 2-3 years from diagnosis despite treatment with a five-year overall survival of approximately 15-20%³⁰.

Disialoganglioside GD2 was found to be expressed at high levels in all H3K27M+ DIPG including those with the H3F3A K27M mutation (SU-DIPG-6, 13, 17, 25, 29) and the less-common HIST1H3B K27M mutation (SU-DIPG-21); GD2 expression was far lower in histone-3 WT pediatric high-grade gliomas (pHGG), including a case of H3WT DIPG³¹.

Ependymomas are glial tumors that occur in approximately 10-12% of all pediatric CNS tumors. Although five-year survivals are favorable, ependymomas frequently recur later, with poor progression-free and overall-survival rates at 10 and 15 years³². So far, there are no results available on the expression GD2 in glial tumors.

Medulloblastoma (MB) is the most frequent malignant embryonal brain tumor of childhood, accounting for approximately 15-20% of all pediatric CNS tumors²². The World Health Organization (WHO) has classified MB as a grade four tumor, genetically originating in the posterior fossa³³. Histologically, MB is classified in four variants: classic, large cell/anaplastic (LCA), with extensive nodularity (MBEN) and desmoplastic/nodular (DN)^{25, 33, 34}. Molecularly, MB have been shown to comprise multiple subtypes defined with distinct driver events, clinical features and outcomes, leading to their reclassification as comprising four broad groups: wingless-activated (WNT) medulloblastomas, Sonic Hedgehog-activated (SHH) medulloblastomas, Group 3 (G3) and Group 4 (G4). Although subgroups within each subtype have also been shown to exist, the most accepted paradigm in clinical practice are these four subtypes. Nowadays, the mainstay of MB treatment is a combination of surgical resection of the tumor, craniospinal irradiation (avoided in children less than 3-5 years of age), and adjuvant chemotherapy³⁵⁻³⁷. Despite standard intensive care, in the high-risk group about 30-40% still succumb to the tumor^{38, 39}. Furthermore, long-term survivors often experience undesirable side effects that dramatically affect their quality of life. Moreover, patients belonging to distinct MB subgroups show a different outcome although receiving the same therapies^{34, 40}. In particular, the WNT subgroup, which accounts for 90% of the 5-year survival rate in MB pediatric populations, has a very good prognosis compared to other MB subgroups^{33, 41, 42}. SHH subgroup has an overall survival (OS) of about 76%⁴²; G3 subgroup is associated with the worst prognosis presenting an OS rate of about 50% of treated patients; G4 subgroup presents a general good prognosis⁴². However, as far as we know about the biology of paediatric MB, significant challenges remain to translate this knowledge into novel therapeutic approaches.

To date, very few data have been published regarding the expression of GD2 in the MB⁴³.

Brain atypical teratoid/rhabdoid tumor (AT/RT) is a highly aggressive CNS embryonal tumor defined by loss of function alterations in the SWI/SNF-related, matrix associated, actin-dependent regulator of chromatin, subfamily B, member 1 (SMARCB1) gene⁴⁴. While AT/RT is the most common malignant CNS tumor in children aged <1, cases in adults (i.e., age >18) are rare⁴⁵. AT/RT are associated with poor survival. There are no results available on the expression GD2 in AT/RT.

Embryonal tumor with multi-layered rosettes (ETMRs) is an aggressive, WHO-grade IV, pediatric CNS tumor most commonly seen in infants and young children (usually less than 3 years old) that were historically classified as CNS primitive neuroectodermal tumors. Most children survive less than a year regardless of therapy, and the reported five-year overall survival is less than 30% 46. So far, there are no results available on the expression GD2 in ETMRs.

The use of the chimeric monoclonal anti-GD2 antibodies ch14.18/SP2/0 (dinutuximab) and ch14.18/CHO (dinutuximab beta) is considered to be standard of care in the first-line treatment of children with high-risk neuroblastoma^{47, 48} Moreover, intrathecal and intraventricular administration of anti-GD2 antibody radioconjugates in patients with MB⁴⁹ and metastatic CNS neuroblastoma⁵⁰ has been well-tolerated in clinical trials⁴⁹.

The use of T cells genetically modified to express a chimeric antigen receptor (CAR) is a new promising approach of adoptive T-cell immunotherapy for cancer, combining antigen specificity of a monoclonal antibody (mAb) with effector function, active bio-distribution and long-term persistence of T cells. CARs are then artificial receptors composed of a region targeting a specific antigen linked, through an intracytoplasmic domain, to the T-cell activation domain CD3zeta chain (first-generation CAR). The intracytoplasmic domain can be enriched by either one or two costimulatory molecules (i.e. CD28, OX40, 4-1BB, or other) to generate second- or third-generation CARs, respectively.

In particular, in the past few years, the immunotherapy based on the transfer of T cells engineered to express anti-GD2 chimeric antigen receptors (GD2.CARs) to target tumor cells has emerged as an exciting new approach for several solid cancers, such as neuroblastoma⁵¹, H3K27M-mutated diffuse midline gliomas⁵², and lung cancers⁵³.

Moreover, it has been reported that GD2 is involved in tumor development through the increase of cell proliferation, growth, motility, migration, adhesion, and invasion, in small-cell lung cancers⁵⁴ and breast cancer⁵⁵.

A phase I clinical trial with a 1st generation of anti GD2-CAR T for patients with neuroblastoma showed to be safe with a transient clinical response^{56, 57}. A phase I clinical trial with a third generation GD2-CAR, incorporating both the CD28 and the OX40 costimulatory end domains (GD2-CAR3), for patients with relapsed or refractory NB patients showed to be safe and no dose-limiting toxicities occurred. However, antitumor responses at 6 weeks were modest⁵⁸.

Recently, a phase I clinical trial was also conducted with the same third generation GD2-CAR (incorporating both the CD28 and the OX40 costimulatory end domains) to treat metastatic melanoma patients in combination with BRAF/MEK inhibitor therapy, and as a monotherapy in patients with colorectal cancer and a patient with fibromyxoid sarcoma. No dose-limiting toxicities or severe adverse events were reported. However, limited clinical effect was reported⁵⁹. Although it has been reported that a normal brain expresses low levels of GD2, in both pre-clinical models⁵¹ and clinical trials, exploring the safety and efficacy of GD2-CAR T-cell whose single chain variable fragment (scFv) being derived from 14g2a monoclonal antibody (including our academic clinical trial NCT03373097), no cases of significant neurotoxicity were reported⁵⁶.

Recently Mount et al. showed, that intravenously administered second generation of GD2-CAR incorporates the single-chain variable fragment derived from the 14g2a monoclonal antibody, a CD8 transmembrane domain and 4-1BB (as co-stimulatory domains) and T cell receptor ζ signalling end-domains are able to cross the BBB and clear patient-derived H3K27M-mutated diffuse midline gliomas (DMG) tumors³¹.

Shumet al. showed that GD2.CAR T cells also engineered to express IL-7 receptor (GD2-CAR.C7R) promotes CAR T cells persistence, proliferation and anti-tumor activity in an orthotopic Glioblastoma (GBM) xenograft model⁶⁰. As an additional safety measure, they generated T cells co-expressing a clinically validated inducible caspase 9 (iC9) suicide gene that can electively eliminate T cells. After double transduction with iC9 and GD2-CAR. C7R retroviral vector, T cells remained sensitive to iC9 signalling and underwent apoptosis in vitro within 24 hours of exposure to the chemical inducer of dimerization AP20187⁶⁰.

All these studies have fastened the clinical translation of CAR.GD2 T cells in patients with Glioma (NCT04099797, NCT04196413).

Table 1 summarizes the known gene therapy products (GD2.CAR) developed for preclinical models of brain tumors (Table 1).

TABLE 1

| Platform | CAR | Single | trackable | Other | | Costimulatory | Brain' | Reference |
|---|---|---|---|---|---|---|---|---|
| Vector | generation | Chain | marker | Gene | TM | domains | s tumours | PMID |
| Retroviral | Second | 14g2a | ScFv | | CD8a | 4-1BB | DIPG | 29662203 |
| Retroviral | Second | 14g2a | ScFv | CR7 | CD8a | 4-1BB | Glioblastoma | 28830878 |

Regarding the first CAR described in Table 1, this CAR does not include any inducible suicide gene as an additional safety switch.

Regarding the second CAR described in Table 1, in this work, in order to obtain T cells that express both CAR-GD2 and the inducible suicide gene, the authors made a double co-transduction, i.e. they used two retroviral vectors. This approach involves the generation of T cells expressing either only the CAR or only the inducible suicide gene, or both. The disadvantage of this approach is that in case of toxicity there is the risk of not being able to control the cells effectively.

With regards to solid tumours, a pre-clinical study published by the inventors showed that third generation GD2.CAR incorporating CD28.4-1BB costimulatory domains is associated with improved anti-tumor efficacy against Neuroblastoma models and long-term persistence in vivo neuroblastoma xenograft mouse models, as compared with a GD2.CAR incorporating the combination of CD28.OX40 or OX40 and 4-1BB domains. In particular, it was demonstrated that the choice of 4-1BB signalling results into significant amelioration of several CAR T-cell characteristics, including: 1) T-cell exhaustion, 2) basal T-cell activation, 3) in vivo tumor control and 4) T-cell persistence. In addition, an improved persistence of IIICAR. GD2 T cells (third generation CAR-GD2 T cells) was observed with respect to IICAR. GD2 T cells (including as costimulatory molecule either CD28, or OX40 or 4-1BB) in in vivo mouse model. In fact, at day 30 the number of IICAR.GD2 T was significantly lower than the number of IIICAR. GD2 T cells. Moreover, the fine-tuning of T-cell culture conditions obtained using IL7 and IL15 was found to be synergic with the GD2.CAR design in increasing the anti-tumor activity of CAR T cells⁵¹. These CAR.GD2 T-cells of third generation were pre-clinically, in both neuroblastoma^{51, 61, 62} and high-grade glioma models⁶³. Based on the above-mentioned preclinical data⁵¹, an academic phase I/II clinical trial was started in 2017 using CAR.GD2 T-cells to treat pediatric patients affected by high-risk and/or relapsed/refractory neuroblastoma or other extracranial GD2-positive solid tumors, specifically GD2+ sarcoma (NCT03373097). The clinical response in neuroblastoma patients has been very promising for both the safety profile of infused CAR. GD2 T cells and for the therapeutic efficacy⁶⁴.

In the light of the above, it is apparent the need to provide for further GD2.CAR T-cells, which are able to overcome the disadvantages of the known GD2.CAR T-cells.

According to the present invention, novel GD2-specific chimeric antigen receptors (GD2.CAR) of third generation and related uses are provided.

In particular, according to the present invention it has surprisingly been found that specific third generation GD2-specific chimeric antigen receptors (GD2.CAR) are able to treat solid tumors such as brain tumors. In this regard, experimental results are provided below concerning efficacy and safety of use of third generation GD2-specific chimeric antigen receptors (GD2.CAR) according to the invention to treat solid tumors, such as sarcoma and medulloblastoma.

In addition, according to the present invention, it has been surprisingly found that the combination of an Enhancer of Zeste Homolog 2 (EZH2) inhibitor with a GD2.CAR according to the invention, in particular a pre-treatment with EZH2 inhibitor followed by the administration of GD2.CAR, can be used in the treatment of tumours which do not express or poorly express GD2 protein, i.e. in tumors against which GD2.CAR alone is not effective.

With regards to the structure of the GD2.CAR according to the invention, in the experimental reported below vectors have been designed with a SFG backbone using uses a Moloney murine leukemia virus (MoMLV)-based retroviral vector. All of env and gag-pol were removed except for the packaging sequence (psi). As a result, the vector produced according to the invention was replication incompetent. Particularly, according to the invention, the following clinical grade third generation retroviral vector (SFG) of GD2.CAR has been produced: SFG.iC9.2A.CAR-GD2 (14g2a) CD8tm.CD28.41BBζ (here after iC9.2A.GD2.CAR-28.4-1BB.ζ), which comprises:
an inducible Caspase 9 (iC9) suicide gene as safety switch⁶⁵
T2A self-cleaving peptides sequence, which can induce ribosomal skipping during translation of a protein in a cell;
a signal peptide;
a single chain variable fragment (scFv) from 14g2a;
a hinge sequence;
a link domain of 3 amino acid (aa);
a transmembrane domain from the transmembrane domain of CD28tm to improve molecule stabilization;
two costimulatory domains were added to the GD2.CAR vector: CD28 and 4-1BB⁶⁶ fused respectively to CD3-3 chain.

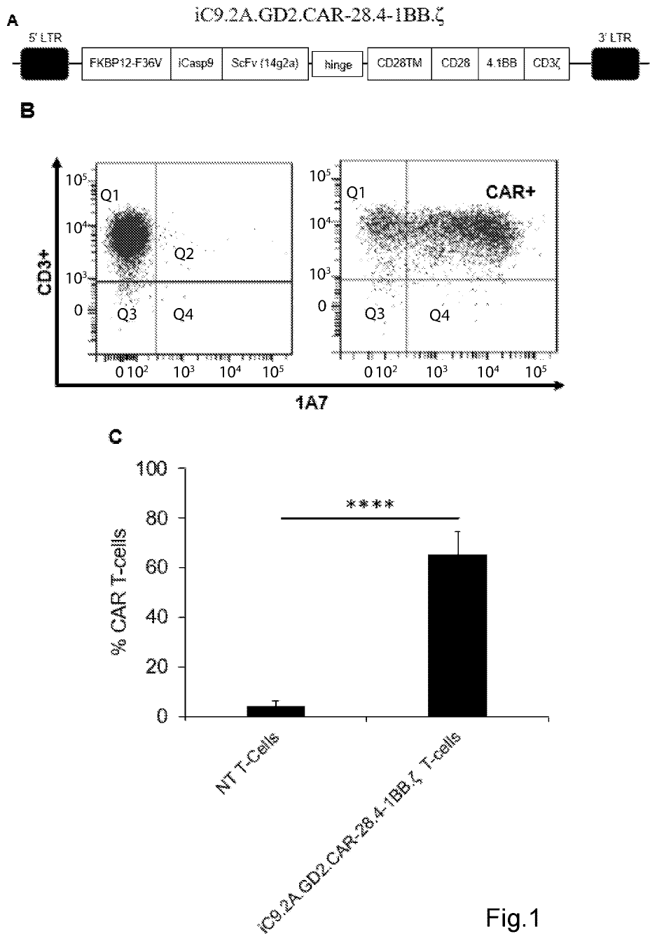

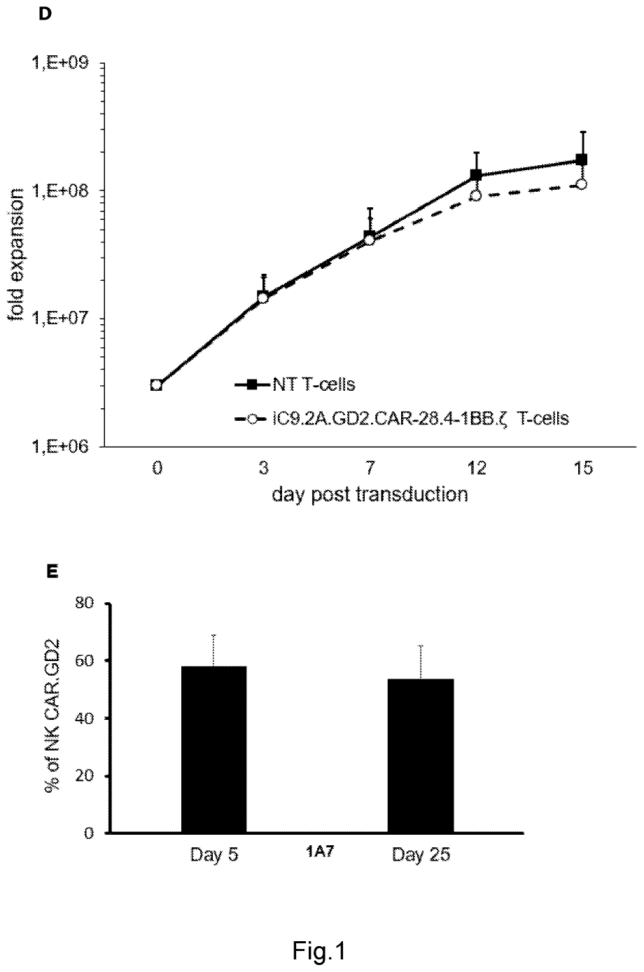

More specifically, the inventors have designed the bicistronic vector shown in FIG. 1A (SFG.iC9.2A.CAR-GD2(14g2a)CD8tm.CD28.41BBζ(iC9.2A.GD2.CAR-28.4-1BB.ζ)), allowing the simultaneous expression of two transgenes, namely the inducible Caspase 9 (iC9) and the third generation GD2.CARs.

Table 2 shows the peculiar elements present in the new GD2.CAR (iC9.2A.GD2.CAR-28.4-1BB.ζ) according to the present invention in comparison with known GD2.CAR reported in table 1, in particular the presence of an inducible suicide gene and the presence of the co-stimulatory domain CD28.4-1BB.

TABLE 2

| Platform | CAR | Single | trackable | Other | | Costimulatory | signalling | Brain' s |
|---|---|---|---|---|---|---|---|---|
| Vector | generation | Chain | marker | Gene | TM | domains | chain | tumours |
| Retroviral | Third | 14g2a | ScFv | Suicide | CD8a | CD28.4-1BB | CD37 | Medulloblastoma |
| | | | | gene | | | | DIPG |
| | | | | (IC9) | | | | |

More in detail, according to the present invention, the inventors generated a clonal retroviral producer cell line producing high title of retroviral vector containing:
5′ LTR—Retroviral long terminal repeats at 5′ end of vector (it functions as promoter sequence);
ψ—Retroviral encapsidation signal (psi; necessary for the packaging of RNA into virion particles);
SA—splice acceptor site;
iCasp9—the inducible caspase-9 expression cassette. iCasp9 consists of the human FK506-binding protein (FKBP12) with an F36V mutation, connected via a 6 amino-acid Gly-Ser linker to a modified CARD domain-deleted human caspase-9:
   FKBP12-F36V—an engineered FK506-binding protein containing F36V mutation to optimize binding affinity for AP1903. The FKBP12-F36V protein domain serves as the drug-binding/oligomerization domain of linked therapeutic proteins (AP1903). FKBP12-F36V functions as a regulator of caspase-9: in the absence of AP1903, iCasp9 has minimal activity; AP1903 binding to FKBP12-F36V promotes dimerization and brings two caspase-9 molecules into apposition to initiate apoptosis. Thus, the FKBP12-F36V moiety functionally replaces the endogenous dimerization/activation module (Caspase Activation and Recruitment Domain; CARD) of caspase-9 that mediates Apaf-1-associated oligomerization;
   Linker—synthetic Ser-Gly-Gly-Gly-Ser-Gly peptide linker used to fuse switch-regulator sequences to caspase-9;
   Caspase-9—Human caspase-9 cDNA sequence (critical pro-apoptotic regulator) and therapeutic component of the construct (regulated suicide gene). The endogenous dimerization/activation module (Caspase Activation and Recruitment Domain; CARD) was deleted to reduce spontaneous Apaf1-binding and hence background killing⁶⁵.
2A—encodes a synthetic 20 amino acid peptide from Thosea Asigna insect virus, which functions as a cleavable linker between the caspase-9 protein and CAR proteins;
Signal peptide—short amino acid sequence to allow the correct translocation of the secretory proteins from the Endoplasmic Reticulum to the cellular membrane;
CAR—CAR molecule comprising
   the single chain variable fragment (scFv) of the fused VH-VL region of the monoclonal antibody 14.G2A specific for the human antigen GD2, in frame with
   the hinge,
   a transmembrane domain from the transmembrane domain of CD8 (CD28 TM),
   a costimulatory domain comprising 4.1bb costimulatory domain and
   CD3ζ cytoplasmic domain.
3′ LTR—Retroviral long terminal repeats at 3′ end of vector (functions as terminator/polyadenylation sequences).

Table 3 shows the functional elements of iC9.2A.GD2.CAR-28.4-1BB.ζ Retroviral Vector designed according to the present invention:

TABLE 3

| Component | Start | End |
|---|---|---|
| LTR | 1 | 590 |
| iCasp9 cassette | 1880 | 3085 |
| FKBP12-F36V | 1880 | 2218 |
| Linker peptide | 2219 | 2236 |
| Casp9 | 2237 | 3085 |
| T2A peptide (2A) | 3086 | 3145 |
| CAR cassette | 3146 | 4723 |
| Signal peptide | 3146 | 3208 |
| scFv (14g2a) | 3209 | 3949 |
| hinge | 3950 | 4045 |
| CD28TM | 4055 | 4135 |
| CD28 | 4136 | 4258 |
| 4.1BB | 4259 | 4384 |
| CD3ζ | 4385 | 4723 |
| LTR | 4896 | 5485 |

The above-mentioned sequence according to the present invention provides unexpected advantages. In fact, according to the experimental results reported below, GD2.CAR-28.4-1BBζ (GD.CAR) T-cells according to the present invention significantly controlled tumor growth of sarcoma cells with high GD2 expression (in particular osteosarcoma (OS) cell lines 143B, MG-63 and U-20S and embryonal rhabdomyosarcoma (ERMS) cell line RD) and, surprisingly, they were also able to significantly control (although partially) tumor growth of sarcoma cell lines with lower expression of GD2, such as the OS cell line HOS, the Alveolar Rhabdomyosarcoma (ARMS) cell line RH41 and the Ewing sarcoma cell line A-673. In addition, as shown by the experimental results reported below, CAR.GD2 T-cells according to the invention exerted effective antitumor activity toward brain tumors as human GD2+ medulloblastoma (MB) cell lines.

In addition, according to the present invention, it has been shown in vivo that the survival of RMS-tumor-bearing mice treated with GD2.CAR-28.4-1BBζ T-cells was significantly longer compared to mice treated with the control. In addition, a long-lasting persistence of T cell up to 75 days was observed. In this regard, it is noted that there are no data in the prior art literature regarding effective GD2.CAR T cell control of soft sarcomas such as rhabdomyosarcomas.

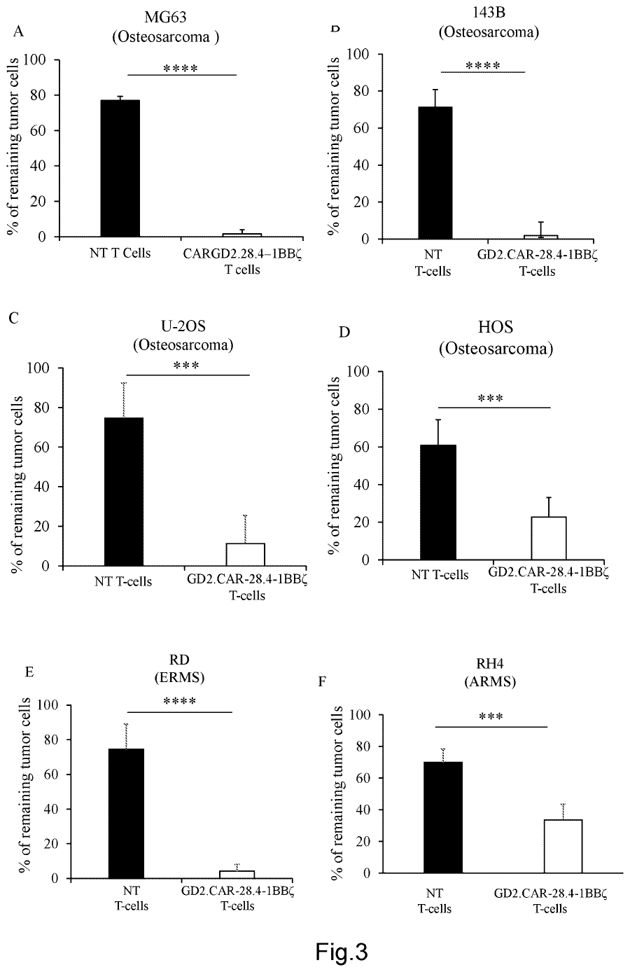

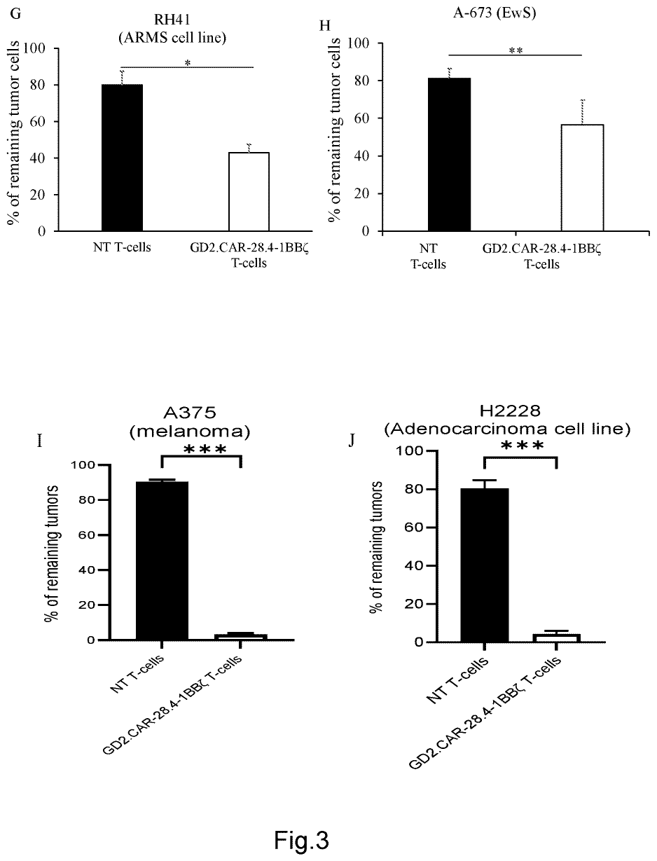

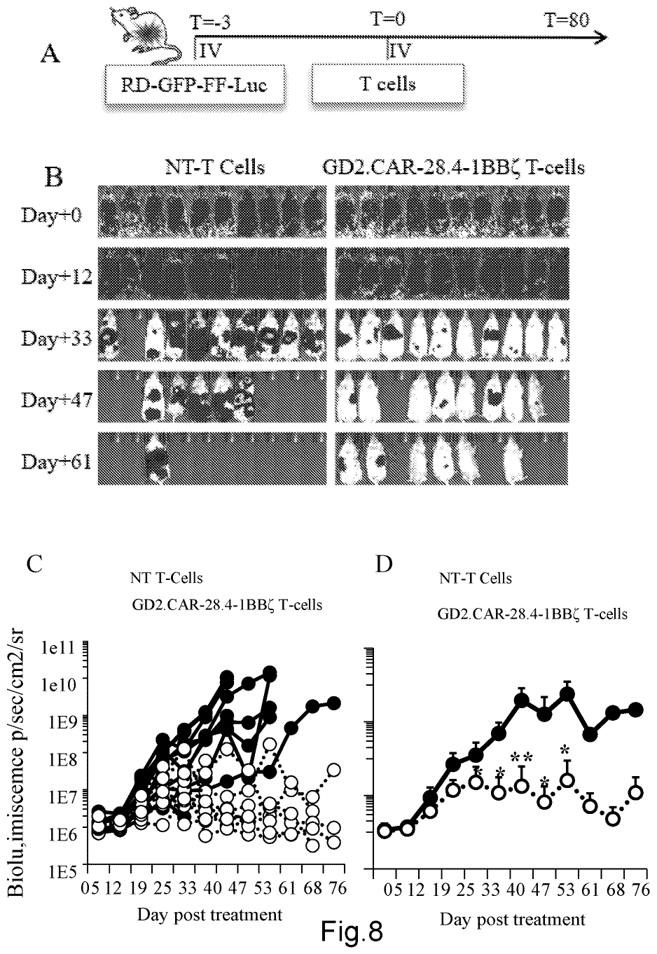

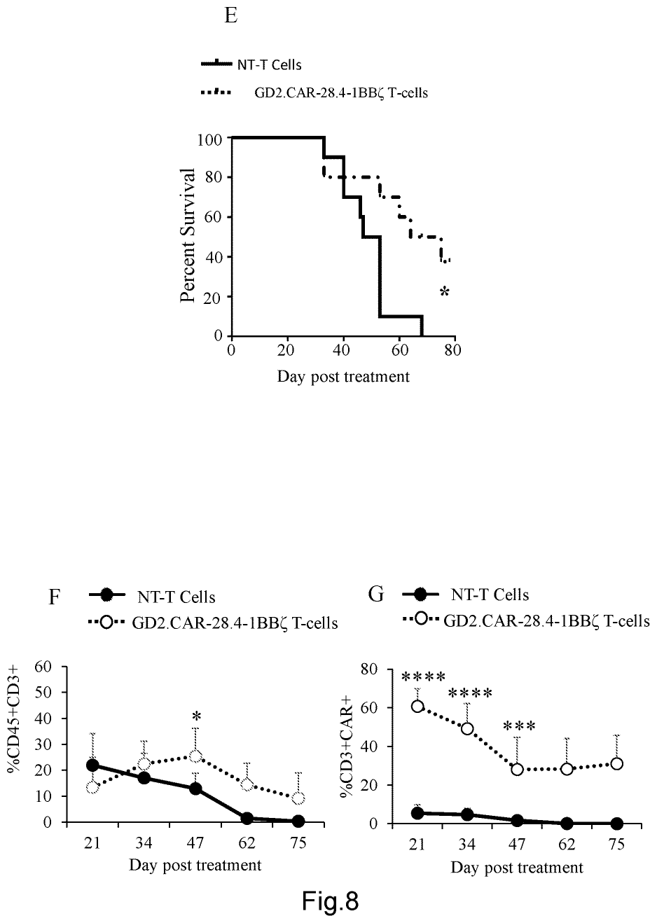

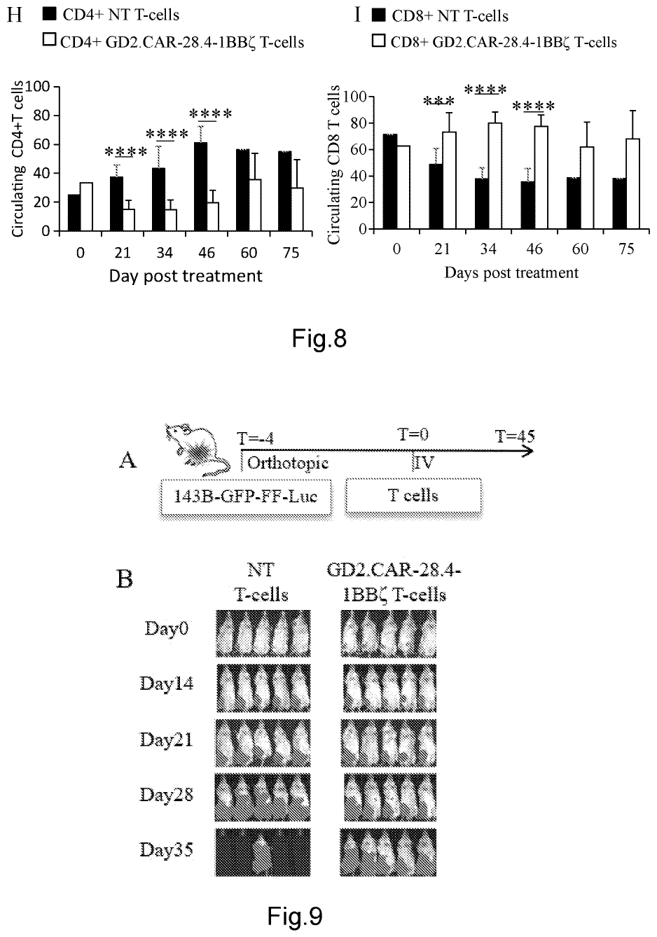

According to the present invention, in vivo data was produced also for OS, showing that GD2.CAR-28.4-1BBζ (GD2.CAR) T-cells significantly slowed down the tumor growth of primary tumor, increasing significantly the overall survival of the mice treated with CAR T-cells respect to NT T-cells. In this regard, it is noted that, by comparing the overall survival of OS mice treated with the GD2.CAR of the invention (FIG. 8E) to that reported in the literature by Adrienne H Long et al (see page 873, FIG. 3B on Cancer Immunology Res; 4 (10) October 2016), it can be deduced that GD2.CAR-28.4-1BBζ T-cells according to the invention show to be superior with respect to GD2-CAR incorporating the 14g2a-scFv with the CD28, OX40, and CD3zeta signalling domains (14g2a.CD28.0X40.ζ) described by Adrienne H Long et al⁶⁷.

In addition, according to the present invention it has been shown that CAR.GD2 T-cells exert an effective antitumor activity toward human GD2+ MB cell lines and in a xenogeneic mouse model and in a patient-derived xenograft mouse model of MB.

Moreover, as stated above, according to the present invention it has been surprisingly found that the combination of an Enhancer of Zeste Homolog 2 (EZH2) inhibitor with a GD2.CAR according to the invention can be used in the treatment of tumours which do not express or poorly express GD2 protein, i.e. in tumors against which GD2 alone is not effective. In particular, according the present invention it has been found that the inhibition of EZH2, by using the EZH2 inhibitor Tazemetostat, up-regulates GD2 expression in Ewing sarcoma, ARMS sarcoma, osteosarcoma and medulloblastoma cell lines and increase their sensibility to GD2.CAR T-cell killing activity.

As reported above, it was known that EZH2 inhibitor up-regulates GD2 expression in Ewing sarcoma. However, according to the above-mentioned articles^{21, 67}, an expert in the field would not be induced to use the EZH2 inhibitor in combination with an anti-GD2 CAR in solid tumors (such as RMS and OS), in particular in tumors that do not have the EWSR1-FLI1 fusion gene. In fact, as stated above, it was reported that the use of the inhibition of EZH2 failed to increase GD2 expression in OS cell lines²¹.

According to the present invention, it has been surprisingly found also that GD2.CAR-28.4-1BBζ T-cells significantly control also the growth of adult aggressive tumours, such as melanoma and lung adenocarcinoma.

It is therefore specific object of the present invention an anti-GD2 chimeric antigen receptor comprising or consisting of, from the N-terminus to the C-terminus:
a) a signal peptide,
b) an anti GD2 single chain antibody domain,
c) a hinge,
d) a trans membrane domain,
e) at least two co-stimulatory signalling domains, and
f) CD3Zeta chain sequence,
for use in the treatment of solid tumors,
wherein said anti GD2 single chain antibody domain comprises or consists of anti GD2 VL sequence and anti GD2 VH sequence linked each other by a linker; and
wherein said solid tumor is not neuroblastoma, diffuse midline glioma (DMG) H3K27M-mutant or osteosarcoma.

In addition, according to an embodiment of the invention, said solid tumor is not high grade glioma. According to a further embodiment of the invention, said solid tumor is not rhabdomyosarcoma or Ewing sarcoma. According to an embodiment of the invention, said solid tumor is not sarcoma. According to a further embodiment of the present invention, said solid tumor is not medulloblastoma.

According to an embodiment of the invention, said tumor is not glioma.

According to the present invention, the solid tumors to be treated are tumors expressing GD2 or tumors expressing GD2 after a treatment with a compound able to enhance the expression of GD2, such as an Enhancer of Zeste Homolog 1 or 2 (EZH2) inhibitor.

According to the present invention, said solid tumors can be chosen from brain tumors, excluding diffuse midline glioma (DMG) H3K27M-mutant, such as diffuse intrinsic pontine gliomas (DIPG) different from diffuse midline glioma (DMG) H3K27M-mutant, adult-type diffuse gliomas, pediatric-type diffuse low-grade pediatric-type diffuse high-grade gliomas, astrocytic gliomas, gliomas, medulloblastoma, glioneuronal and neuronal tumors, ependymal tumors, choroid plexus tumors; embryonal tumors, pineal tumors, cranial and paraspinal nerve tumors; extra-cranic tumors, such as sarcomas different from osteosarcoma; retinoblastoma; melanoma; lung cancer; gastric cancer; bladder cancer, head and neck cancer; ovarian cancer; breast cancer pancreatic cancer; colorectal cancer or other tumors expressing GD2.

According to the present invention, said sarcoma can be chosen from rhabdomyosarcoma, in particular Alveolar Rhabdomyosarcoma or embryonal Rhabdomyosarcoma, Ewing's sarcoma (EWS), desmoplastic small round cell tumors, leiomyosarcomas and liposarcomas; and said medulloblastoma can be chosen from SHH, G3, G4 and WNT medulloblastoma subgroups.

According to the present invention, said tumor can be a tumor having an expression of GD2 higher than the expression of GD2 in a negative healthy control, namely healthy peripheral blood mononuclear cells (PBMC), wherein the expression of GD2 is measured as the percentage of GD2+ cells with respect to the total number of cells of the tumor or of the healthy peripheral blood mononuclear cells (the percentage of GD2 positive cells in healthy PBMC being equal to 0.43%±0.90%). Preferably, said tumor is a tumor having an expression of GD2 higher than 2%, i.e. having a percentage of GD2+ cells higher than 2%.

According to an embodiment of the invention, said brain tumor is not high grade glioma. According to a further embodiment of the invention, said sarcoma is not rhabdomyosarcoma or Ewing sarcoma. According to an embodiment of the invention, said solid tumor is not sarcoma. According to a further embodiment of the present invention, said brain tumor is not medulloblastoma.

According to an embodiment of the present invention, said anti GD2 VL sequence can comprise CDR1 sequence QSLVHRNGNTY (SEQ ID NO: 5), CDR2 sequence: KVS and CDR3 sequence SQSTHVP (SEQ ID NO: 7); whereas said anti GD2 VH sequence can comprise CDR1 sequence: GSSFTGYN (SEQ ID NO: 8), CDR2 sequence: IDPYYGGT (SEQ ID NO: 9) and CDR3 sequence: VSGMEY (SEQ ID NO: 10).

In particular, according to the present invention,
anti GD2 14G2a VL sequence can comprises or
consist of:
(SEQ ID NO: 11)
DILLTQTPLSLPVSLGDQASISCRSSQSLVHRNGNTYLHWYLQKPGQSP
KLLIHKVSNRFSGVPDRFSGSGSGTDFTLKISRVEAEDLGVYFCSQSTH
VPPLTFGAGTKLELKRADAAPTVSIFP,
and
anti GD2 14G2a VH sequence can comprise or
consists of:
(SEQ ID NO: 12)
EVKLQQSGPSLVEPGASVMISCKASGSSFTGYNMNWVRQNIGKSLEWIG
AIDPYYGGTSYNQKFKGRATLTVDKSSSTAYMHLKSLTSEDSAVYYCVS
GMEYWGQGTSVTVSS.

In particular, sequence SEQ ID NO: 11 comprises CDR1 in position 27-37, CDR2 in position 55-57 and CDR3 in position 94-10, whereas sequence SEQ ID NO: 12 comprises CDR1 in position 26-33 and CDR2 in position 51-58 and CDR3 in position 97-102.

According to the present invention, the linker which links anti GD2 VL sequence and anti GD2 VH sequence can be a short flexible glycines-rich linker with a length from 7 to 14 amino acids, such as from 7 to 12, from 7 to 10 or 8 amino acids, for example said linker being selected from G7S2 linker GSGGGGSGG (SEQ ID NO: 13), (G4S) 2 linker GGGGSGGGG (SEQ ID NO: 14), G4SG2 linker GGGGSGG (SEQ ID NO: 15), G3SG4 linker GGGSGGGG (SEQ ID NO: 16), SG4SG3 linker SGGGGSGGG (SEQ ID NO: 17), (SG4) 2 S linker SGGGGSGGGGS (SEQ ID NO: 18), (SG4) 2 SG linker SGGGGSGGGGSG (SEQ ID NO: 19), (SG4) 2 SG3 linker SGGGGSGGGGSGGG linker (SEQ ID NO: 20), (SG4) 2 SGGGGSGGGG (SEQ ID NO: 21), or (SG4) 2 SG2 SGGGGSGGGGSGG (SEQ ID NO: 22), preferably G7S2 linker GSGGGGSGG (SEQ ID NO: 13).

Therefore, according to the present invention, the anti GD2 single chain antibody domain can be DILLTQTPLSLPVSLGDQASISCRSSQSLVHRNGNTYLHWYLQKPGQSPKLLIHK VSNRFSGVPDRFSGSGSGTDFTLKISRVEAEDLGVYFCSQSTHVPPLTFGAGTK LELKRADAAPTVSIFPGSGGGGSGGEVKLQQSGPSLVEPGASVMISCKASGSSF TGYNMNWVRQNIGKSLEWIGAIDPYYGGTSYNQKFKGRATLTVDKSSSTAYMHL KSLTSEDSAVYYCVSGMEYWGQGTSVTVSS (SEQ ID NO: 4).

According to the present invention, said hinge can comprise or consists of one or more of the following hinges:

IgG-based hinge of sequence
(SEQ ID NO: 23)
AKTTPPSVYGRVTVSSAEPKSCDKTHTCPPCP;
hinge Spacer-CD8α of sequence
(SEQ ID NO: 24)
PAPRPPTPAPTIASQPLSLRPEACRPAAGGAVHTRGLDFA
(nucleotide ID NO: M12828.1 and Protein ID NO: AAB04637.1);
CD8stalk of sequence
(SEQ ID NO: 25)
TTTPAPRPPTPAPTIASQPLSLRPEACRPAAGGAVHTRGLDFACD;
hinge CD28 of sequence
(SEQ ID NO: 26)
EVMYPPPYLDNEKSNGTIIHVKGKHLCPSPLFPGPSKP;
hinge CH2-CH3 of sequence
(SEQ ID NO: 27)
ESKYGPPCPSCPAPEFLGGPSVFLFPPKPKDTLMISRTPEVTCVVVDVSQEDPE
VQFNWYVDGVEVHNAKTKPREEQFNSTYRVVSVLTVLHQDWLNGKEYKCKVSN
KGLPSSIEKTISKAKGQPREPQVYTLPPSQEEMTKNQVSLTCLVKGFYPSDIAVE
WESNGQPENNYKTTPPVLDSDGSFFLYSRLTVDKSRWQEGNVFSCSVMHEALH
NHYTQKSLSLSLGK;
hinge CH3 of sequence
(SEQ ID NO: 28)
ESKYGPPCPSCPGQPREPQVYTLPPSQEEMTKNQVSLTCLVKGFYPSDIAVEW
ESNGQPENNYKTTPPVLDSDGSFFLYSRLTVDKSRWQEGNVFSCSVMHEALHN
HYTQKSLSLSLGK;

preferably IgG-based hinge of sequence SEQ ID NO: 23.

According to the present invention, said trans membrane domain of anti-GD2 chimeric antigen receptor can be chosen from the group consisting of CD8aTM: CDIYIWAPLAGTCGVLLLSLVIT (SEQ ID NO: 6), (nucleotide ID NO NM_001768.6 and Protein ID NO: NP_001759.3); CD28TM: FWVLVVVGGVLACYSLLVTVAFIIFWV (SEQ ID NO: 29) (nucleotide ID NO: BC112085.1 and Protein ID NO: AAI12086.1); preferably is CD28TM of sequence FWVLVVVGGVLACYSLLVTVAFIIFWV (SEQ ID NO: 29).

According to the present invention, said hinge can be linked to said transmembrane domain by a linker (or connection sequence); preferably said linker has three amino acids; more preferably said linker has sequence DPK.

According to the present invention, said two co-stimulatory signalling domains can be chosen from the group consisting of:
a sequence obtained by linking:
CD28 cytoplasmic sequence:
RSKRSRLLHSDYMNMTPRRPGPTRKHYQPYAPPRDFAAYRS (SEQ ID NO: 30) to
OX40 sequence:
RDQRLPPDAHKPPGGGSFRTPIQEEQADAHSTLAKI (SEQ ID NO: 32), wherein CD28 cytoplasmic sequence is positioned before or after OX40 sequence;
a sequence obtained by linking:
CD28 cytoplasmic sequence:

RSKRSRLLHSDYMNMTPRRPGPTRKHYQPYAPPRDFAAYRS (SEQ ID NO: 30) to
CD137 (4-1BB) sequence:
KRGRKKLLYIFKQPFMRPVQTTQEEDGCSCRFPEEEEGGCEL (SEQ ID NO: 31), wherein CD28 cytoplasmic sequence is positioned before or after CD137 (4-1BB) sequence;
a sequence obtained by linking:
OX40 sequence:
RDQRLPPDAHKPPGGGSFRTPIQEEQADAHSTLAKI (SEQ ID NO: 32) to CD28 cytoplasmic sequence:
RSKRSRLLHSDYMNMTPRRPGPTRKHYQPYAPPRDFAAYRS (SEQ ID NO: 30), wherein OX40 sequence is positioned before or after CD28 cytoplasmic sequence; or
a sequence obtained by linking:
OX40 sequence:
RDQRLPPDAHKPPGGGSFRTPIQEEQADAHSTLAKI (SEQ ID NO: 32) to
CD137 (4-1BB) sequence:
KRGRKKLLYIFKQPFMRPVQTTQEEDGCSCRFPEEEEGGCEL (SEQ ID NO: 31), wherein OX40 sequence is positioned before or after CD137 (4-1BB) sequence;
preferably a sequence obtained by linking:
CD28 cytoplasmic sequence:
RSKRSRLLHSDYMNMTPRRPGPTRKHYQPYAPPRDFAAYRS (SEQ ID NO: 30), to
CD137 (4-1BB) sequence:
KRGRKKLLYIFKQPFMRPVQTTQEEDGCSCRFPEEEEGGCEL (SEQ ID NO: 31), wherein CD28 cytoplasmic sequence is positioned before CD137 (4-1BB) sequence.

According to the present invention, the CD3-Zeta chain sequence can have the following sequence:

(SEQ ID NO: 53)
RVKFSRSADAPAYQQGQNQLYNELNLGRREEYDVLDKRRGRDPE
MGGKPRRKNPQEGLYNELQKDKMAEAYSEIGMKGERRRGKGHDG
LYQGLSTATKDTYDALHMQALPPR*
(nucleotide ID NO: J04132.1 And Protein ID:
AAA60394.1).

According to the present invention, said anti-GD2 chimeric antigen receptor can further comprise cytoplasmic moiety of CD8cyt, CD8a cytoplasmic (CD8a cyto, of sequence LYCNHRN (SEQ ID NO: 51) between the trans membrane domain and the co-stimulatory signalling domain.

According to the present invention, said signal peptide can comprise or consist of the sequence MEFGLSWLFLVAILKGVQCSR (SEQ ID NO: 3).

According to a specific embodiment of the present invention, said anti-GD2 chimeric antigen receptor can comprise or consist of the following sequence:

GD2.CAR-28.4-1BB.ζ
(SEQ ID NO: 39)
MEFGLSWLFLVAILKGVQCSRDILLTQTPLSLPVSLGDQASISC
RSSQSLVHRNGNTYLHWYLQKPGQSPKLLIHKVSNRFSGVPDRF
SGSGSGTDFTLKISRVEAEDLGVYFCSQSTHVPPLTFGAGTKLE
LKRADAAPTVSIFPGSGGGGSGGEVKLQQSGPSLVEPGASVMIS
CKASGSSFTGYNMNWVRQNIGKSLEWIGAIDPYYGGTSYNQKFK
GRATLTVDKSSSTAYMHLKSLTSEDSAVYYCVSGMEYWGQGTSV
TVSSAKTTPPSVYGRVTVSSAEPKSCDKTHTCPPCPDPKFWVLV
VVGGVLACYSLLVTVAFIIFWVRSKRSRLLHSDYMNMTPRRPGP
TRKHYQPYAPPRDFAAYRSKRGRKKLLYIFKQPFMRPVQTTQEE
DGCSCRFPEEEEGGCELRVKFSRSADAPAYQQGQNQLYNELNLG
RREEYDVLDKRRGRDPEMGGKPRRKNPQEGLYNELQKDKMAEAY
SEIGMKGERRRGKGHDGLYQGLSTATKDTYDALHMQALPPR*.
In particular, sequence SEQ ID NO: 39
comprises
a Signal peptide:
(SEQ ID NO: 3)
MEFGLSWLFLVAILKGVQCSR;
anti GD2(14G2a) VL sequence:
(SEQ ID NO: 11)
DILLTQTPLSLPVSLGDQASISCRSSQSLVHRNGNTYLHWYLQK
PGQSPKLLIHKVSNRFSGVPDRFSGSGSGTDFTLKISRVEAEDL
GVYFCSQSTHVPPLTFGAGTKLELKRADAAPTVSIFP.
G7S2 linker:
(SEQ ID NO: 13)
GSGGGGSGG
anti GD2(14G2a) VH sequence:
(SEQ ID NO: 12)
EVKLQQSGPSLVEPGASVMISCKASGSSFTGYNMNWVRQNIGKS
LEWIGAIDPYYGGTSYNQKFKGRATLTVDKSSSTAYMHLKSLTS
EDSAVYYCVSGMEYWGQGTSVTVSS.
IgG-based hinge:
(SEQ ID NO: 23)
AKTTPPSVYGRVTVSSAEPKSCDKTHTCPPCP
which is linked by the Linker (connection
sequence) DPK to a transmembrane domain:
CD28TM:
(SEQ ID NO: 29)
FWVLVVVGGVLACYSLLVTVAFIIFWV
CD28 cytoplasmic sequence:
(SEQ ID NO: 30)
RSKRSRLLHSDYMNMTPRRPGPTRKHYQPYAPPRDFAAYRS
CD137 (4-1BB) sequence:
(SEQ ID NO: 31)
KRGRKKLLYIFKQPFMRPVQTTQEEDGCSCRFPEEEEGGCEL
CD3-Zeta chain:
(SEQ ID NO: 53)
RVKFSRSADAPAYQQGQNQLYNELNLGRREEYDVLDKRRGRDPE
MGGKPRRKNPQEGLYNELQKDKMAEAYSEIGMKGERRRGKGHDG
LYQGLSTATKDTYDALHMQALPPR*.

It is a further object of the present invention a nucleotide sequence or vector comprising the nucleotide sequence, said nucleotide sequence comprising or consisting of a nucleotide sequence which encodes an anti-GD2 chimeric antigen receptor as defined above for use in the treatment of solid tumors, wherein
said solid tumor is not neuroblastoma, diffuse midline gliomas (DMG) H3K27M-mutant or osteosarcoma.

In addition, according to an embodiment of the invention, said solid tumor is not high grade glioma. According to a further embodiment of the invention, said solid tumor is not rhabdomyosarcoma or Ewing sarcoma. According to an embodiment of the invention, said solid tumor is not sarcoma.

According to a further embodiment of the present invention, said solid tumor is not medulloblastoma. According to an embodiment of the invention, said tumor is not glioma.

As stated above, according to the present invention, the solid tumors to be treated are tumors expressing GD2 or tumors expressing GD2 after a treatment with a compound able to enhance the expression of GD2, such as an Enhancer of Zeste Homolog 1 or 2 (EZH2) inhibitor.

As stated above, according to the present invention, said solid tumors can be chosen from brain tumors, excluding diffuse midline gliomas (DMG) H3K27M-mutant, such as diffuse intrinsic pontine gliomas (DIPG) different from diffuse midline glioma (DMG) H3K27M-mutant, adult-type diffuse gliomas, pediatric-type diffuse low-grade gliomas, pediatric-type diffuse high-grade gliomas, astrocytic gliomas, medulloblastoma; glioneuronal and neuronal tumors; ependymal tumors; choroid plexus tumors; embryonal tumors, medulloblastoma, pineal tumors, cranial and paraspinal nerve tumors; extra-cranic tumors, such as sarcomas different from osteosarcoma; retinoblastoma; melanoma; lung cancer; gastric cancer; bladder cancer, head and neck cancer; ovarian cancer; breast cancer; pancreatic cancer; colorectal cancer or other tumors expressing GD2.

According to the invention, said sarcoma can be chosen from osteosarcoma, rhabdomyosarcoma, in particular Alveolar Rhabdomyosarcoma or embryonal Rhabdomyosarcoma, Ewing's sarcoma (EWS), desmoplastic small round cell tumors, leiomyosarcomas and liposarcomas; and said medulloblastoma can be chosen from SHH, G3, G4 and WNT medulloblastoma subgroups.

As stated above, according to the invention said tumor can be a tumor having an expression of GD2 higher than the expression of GD2 in a negative healthy control, namely peripheral blood mononuclear cells (PBMC), wherein the expression of GD2 is measured as the percentage of GD2+ cells with respect to the total number of cells of the tumor or of the healthy peripheral blood mononuclear cells (the percentage of GD2 positive cells in healthy PBMC being equal to 0.43%±0.90%). Preferably, said tumor is a tumor having an expression of GD2 higher than 2%, i.e. having a percentage of GD2+ cells higher than 2%.

According to an embodiment of the invention, said brain tumor is not high grade glioma. According to a further embodiment of the invention, said sarcoma is not rhabdomyosarcoma or Ewing sarcoma. According to an embodiment of the invention, said solid tumor is not sarcoma. According to a further embodiment of the present invention, said brain tumor is not medulloblastoma.

According to the present invention, said
anti GD2 VL sequence can be encoded by the
nucleotide sequence
(SEQ ID NO: 36)
GATATTTTGCTGACCCAAACTCCACTCTCCCTGCCTGTCAGTCT
TGGAGATCAAGCCTCCATCTCTTGCAGATCTAGTCAGAGTCTTG
TACACCGTAATGGAAACACCTATTTACATTGGTACCTGCAGAAG
CCAGGCCAGTCTCCAAAGCTCCTGATTCACAAAGTTTCCAACCG
ATTTTCTGGGGTCCCAGACAGGTTCAGTGGCAGTGGATCAGGGA
CAGATTTCACACTCAAGATCAGCAGAGTGGAGGCTGAGGATCTG
GGAGTTTATTTCTGTTCTCAAAGTACACATGTTCCTCCGCTCAC
GTTCGGTGCTGGGACCAAGCTGGAGCTGAAACGGGCTGATGCTG
CACCAACTGTATCCATCTTCCCA,
and
said anti GD2 VH sequence is encoded by the
nucleotide sequence
(SEQ ID NO: 37)
GAGGTGAAGCTTCAGCAGTCTGGACCTAGCCTGGTGGAGCCTGG
CGCTTCAGTGATGATATCCTGCAAGGCTTCTGGTTCCTCATTCA
CTGGCTACAACATGAACTGGGTGAGGCAGAACATTGGAAAGAGC
CTTGAATGGATTGGAGCTATTGATCCTTACTATGGTGGAACTAG
CTACAACCAGAAGTTCAAGGGCAGGGCCACATTGACTGTAGACA
AATCGTCCAGCACAGCCTACATGCACCTCAAGAGCCTGACATCT
GAGGACTCTGCAGTCTATTACTGTGTAAGCGGAATGGAGTACTG
GGGTCAAGGAACCTCAGTCACCGTCTCCTCA.

In ((anti GD2 (14G2a)) VL sequence SEQ ID NO: 36 the sequences encoding CDR1, CDR2 and CDR3 are the following:

CDR1 sequence:
(SEQ ID NO: 38)
CAGAGTCTTGTACACCGTAATGGAAACACCTAT
in position 79-111;
CDR2 sequence:
AAAGTTTCC
in position 163-171
and
CDR3 sequence:
(SEQ ID NO: 40)
TCTCAAAGTACACATGTTCCTCC
in position 280-302.

In ((anti GD2 (14G2a)) VH sequence SEQ ID NO: 37 the sequences encoding CDR1, CDR2 and CDR3 are the following:

CDR1 sequence:
(SEQ ID NO: 41)
GGTTCCTCATTCACTGGCTACAAC
in position 76-99
CDR2 sequence:
(SEQ ID NO: 42)
ATTGATCCTTACTATGGTGGAACT
in position 151-174
and
CDR3 sequence:
(SEQ ID NO: 43)
GTAAGCGGAATGGAGTAC
in position 289-306.

According to a specific embodiment of the present invention, the nucleotide sequence encoding anti-GD2 chimeric antigen receptor can be:

GD2.CAR-28.4-1BB.ζ
(SEQ ID NO: 52)
ATGGAGTTTGGGCTGAGCTGGCTTTTTCTTGTGGCTATTTTAAA
AGGTGTCCAGTGCTCTAGAGATATTTTGCTGACCCAAACTCCAC
TCTCCCTGCCTGTCAGTCTTGGAGATCAAGCCTCCATCTCTTGC
AGATCTAGTCAGAGTCTTGTACACCGTAATGGAAACACCTATTT
ACATTGGTACCTGCAGAAGCCAGGCCAGTCTCCAAAGCTCCTGA
TTCACAAAGTTTCCAACCGATTTTCTGGGGTCCCAGACAGGTTC
AGTGGCAGTGGATCAGGGACAGATTTCACACTCAAGATCAGCAG
AGTGGAGGCTGAGGATCTGGGAGTTTATTTCTGTTCTCAAAGTA
CACATGTTCCTCCGCTCACGTTCGGTGCTGGGACCAAGCTGGAG
CTGAAACGGGCTGATGCTGCACCAACTGTATCCATCTTCCCAGG
CTCGGGCGGTGGTGGGTCGGGTGGCGAGGTGAAGCTTCAGCAGT
CTGGACCTAGCCTGGTGGAGCCTGGCGCTTCAGTGATGATATCC
TGCAAGGCTTCTGGTTCCTCATTCACTGGCTACAACATGAACTG
GGTGAGGCAGAACATTGGAAAGAGCCTTGAATGGATTGGAGCTA
TTGATCCTTACTATGGTGGAACTAGCTACAACCAGAAGTTCAAG
GGCAGGGCCACATTGACTGTAGACAAATCGTCCAGCACAGCCTA
CATGCACCTCAAGAGCCTGACATCTGAGGACTCTGCAGTCTATT
ACTGTGTAAGCGGAATGGAGTACTGGGGTCAAGGAACCTCAGTC
ACCGTCTCCTCAGCCAAAACGACACCCCCATCAGTCTATGGAAG
GGTCACCGTCTCTTCAGCGGAGCCCAAATCTTGTGACAAAACTC
ACACATGCCCACCGTGCCCGGATCCCAAATTTTGGGTGCTGGTG
GTGGTTGGTGGAGTCCTGGCTTGCTATAGCTTGCTAGTAACAGT
GGCCTTTATTATTTTCTGGGTGAGGAGTAAGAGGAGCAGGCTCC
TGCACAGTGACTACATGAACATGACTCCCCGCCGCCCCGGGCCC
ACCCGCAAGCATTACCAGCCCTATGCCCCACCACGCGACTTCGC
AGCCTATCGCTCCAAACGGGGCAGAAAGAAACTCCTGTATATAT
TCAAACAACCATTTATGAGACCAGTACAAACTACTCAAGAGGAA
GATGGCTGTAGCTGCCGATTTCCAGAAGAAGAAGAAGGAGGATG
TGAACTGAGAGTGAAGTTCAGCAGGAGCGCAGACGCCCCCGCGT
ACCAGCAGGGCCAGAACCAGCTCTATAACGAGCTCAATCTAGGA
CGAAGAGAGGAGTACGATGTTTTGGACAAGAGACGTGGCCGGGA
CCCTGAGATGGGGGGAAAGCCGAGAAGGAAGAACCCTCAGGAAG
GCCTGTACAATGAACTGCAGAAAGATAAGATGGCGGAGGCCTAC
AGTGAGATTGGGATGAAAGGCGAGCGCCGGAGGGGCAAGGGGCA
CGATGGCCTTTACCAGGGTCTCAGTACAGCCACCAAGGACACCT
ACGACGCCCTTCACATGCAGGCCCTGCCCCCTCGCTAA.

In particular, the sequence SEQ ID NO: 52 comprises:

Signal peptide:
(SEQ ID NO: 35)
ATGGAGTTTGGGCTGAGCTGGCTTTTTCTTGTGGCTATTTTAAA
AGGTGTCCAGTGCTCTAGA,
The G7S2 linker (which links anti GD2 (14G2a)
VL sequence and anti GD2 (14G2a) VH sequence)
sequence:
(SEQ ID NO: 44)
GGCTCGGGCGGTGGTGGGTCGGGTGGC;
IgG-based hinge sequence:
(SEQ ID NO: 45)
GCCAAAACGACACCCCCATCAGTCTATGGAAGGGTCACCGTCTC
TTCAGCGGAGCCCAAATCTTGTGACAAAACTCACACATGCCCAC
CGTGCCCG;
which is linked by the Linker (connection
sequence) GATCCCAAA to a transmembrane domain
CD28TM sequence:
(SEQ ID NO: 46)
TTTTGGGTGCTGGTGGTGGTTGGTGGAGTCCTGGCTTGCTATAG
CTTGCTAGTAACAGTGGCCTTTATTATTTTCTGGGTG;
CD28 cytoplasmic sequence:
(SEQ ID NO: 47)
AGGAGTAAGAGGAGCAGGCTCCTGCACAGTGACTACATGAACAT
GACTCCCCGCCGCCCCGGGCCCACCCGCAAGCATTACCAGCCCT
ATGCCCCACCACGCGACTTCGCAGCCTATCGCTCC
CD137 (4-1BB) sequence:
(SEQ ID NO: 48)
AAACGGGGCAGAAAGAAACTCCTGTATATATTCAAACAACCATT
TATGAGACCAGTACAAACTACTCAAGAGGAAGATGGCTGTAGCT
GCCGATTTCCAGAAGAAGAAGAAGGAGGATGTGAACTG
CD3-Zeta chain sequence:
(SEQ ID NO: 49)
AGAGTGAAGTTCAGCAGGAGCGCAGACGCCCCCGCGTACCAGCA
GGGCCAGAACCAGCTCTATAACGAGCTCAATCTAGGACGAAGAG
AGGAGTACGATGTTTTGGACAAGAGACGTGGCCGGGACCCTGAG
ATGGGGGGAAAGCCGAGAAGGAAGAACCCTCAGGAAGGCCTGTA
CAATGAACTGCAGAAAGATAAGATGGCGGAGGCCTACAGTGAGA
TTGGGATGAAAGGCGAGCGCCGGAGGGGCAAGGGGCACGATGGC
CTTTACCAGGGTCTCAGTACAGCCACCAAGGACACCTACGACGC
CCTTCACATGCAGGCCCTGCCCCCTCGCTAA.

According to an embodiment of the present invention, said nucleotide sequence or vector can further comprise a nucleotide sequence encoding a suicide gene inducible amino acid sequence linked to the 5′ end or 3′ end of the nucleotide sequence encoding said chimeric antigen receptor by a nucleotide sequence encoding a 2A self-cleaving peptide.

In particular, said suicide gene inducible amino acid sequence can comprise or consist of a chimeric Caspase-9 polypeptide or ca herpes simplex virus thymidine kinase (HSV-TK) or ACD19 sequence as a safety switch.

Therefore, in the cell, the polynucleotide 2A selfcleaving peptide cuts the peptide comprising the suicide gene inducible amino acid sequence and the chimeric antigen receptor in two separate peptides, i.e., the suicide gene inducible and the chimeric antigen receptor amino acid sequences.

Therefore, according to the present invention, the nucleotide sequence of anti-GD2 chimeric antigen receptor also comprising a suicide gene inducible sequence can be:

iC9.2A.GD2.CAR-28.4-1BB.ζ
(SEQ ID NO: 50)
ATGCTCGAGGGAGTGCAGGTGGAGACTATCTCCCCAGGAGACGG
GCGCACCTTCCCCAAGCGCGGCCAGACCTGCGTGGTGCACTACA
CCGGGATGCTTGAAGATGGAAAGAAAGTTGATTCCTCCCGGGAC
AGAAACAAGCCCTTTAAGTTTATGCTAGGCAAGCAGGAGGTGAT
CCGAGGCTGGGAAGAAGGGGTTGCCCAGATGAGTGTGGGTCAGA
GAGCCAAACTGACTATATCTCCAGATTATGCCTATGGTGCCACT
GGGCACCCAGGCATCATCCCACCACATGCCACTCTCGTCTTCGA
TGTGGAGCTTCTAAAACTGGAATCTGGCGGTGGATCCGGAGTCG
ACGGATTTGGTGATGTCGGTGCTCTTGAGAGTTTGAGGGGAAAT
GCAGATTTGGCTTACATCCTGAGCATGGAGCCCTGTGGCCACTG
CCTCATTATCAACAATGTGAACTTCTGCCGTGAGTCCGGGCTCC
GCACCCGCACTGGCTCCAACATCGACTGTGAGAAGTTGCGGCGT
CGCTTCTCCTCGCTGCATTTCATGGTGGAGGTGAAGGGCGACCT
GACTGCCAAGAAAATGGTGCTGGCTTTGCTGGAGCTGGCGCgGC
AGGACCACGGTGCTCTGGACTGCTGCGTGGTGGTCATTCTCTCT
CACGGCTGTCAGGCCAGCCACCTGCAGTTCCCAGGGGCTGTCTA
CGGCACAGATGGATGCCCTGTGTCGGTCGAGAAGATTGTGAACA
TCTTCAATGGGACCAGCTGCCCCAGCCTGGGAGGGAAGCCCAAG
CTCTTTTTCATCCAGGCCTGTGGTGGGGAGCAGAAAGACCATGG
GTTTGAGGTGGCCTCCACTTCCCCTGAAGACGAGTCCCCTGGCA
GTAACCCCGAGCCAGATGCCACCCCGTTCCAGGAAGGTTTGAGG
ACCTTCGACCAGCTGGACGCCATATCTAGTTTGCCCACACCCAG
TGACATCTTTGTGTCCTACTCTACTTTCCCAGGTTTTGTTTCCT
GGAGGGACCCCAAGAGTGGCTCCTGGTACGTTGAGACCCTGGAC
GACATCTTTGAGCAGTGGGCTCACTCTGAAGACCTGCAGTCCCT
CCTGCTTAGGGTCGCTAATGCTGTTTCGGTGAAAGGGATTTATA
AACAGATGCCTGGTTGCTTTAATTTCCTCCGGAAAAAACTTTTC
TTTAAAACATCAGCTAGCAGAGCCGAGGGCAGGGGAAGTCTTCT
AACATGCGGGGACGTGGAGGAAAATCCCGGGCCCATGGAGTTTG
GGCTGAGCTGGCTTTTTCTTGTGGCTATTTTAAAAGGTGTCCAG
TGCTCTAGAGATATTTTGCTGACCCAAACTCCACTCTCCCTGCC
TGTCAGTCTTGGAGATCAAGCCTCCATCTCTTGCAGATCTAGTC
AGAGTCTTGTACACCGTAATGGAAACACCTATTTACATTGGTAC
CTGCAGAAGCCAGGCCAGTCTCCAAAGCTCCTGATTCACAAAGT
TTCCAACCGATTTTCTGGGGTCCCAGACAGGTTCAGTGGCAGTG
GATCAGGGACAGATTTCACACTCAAGATCAGCAGAGTGGAGGCT
GAGGATCTGGGAGTTTATTTCTGTTCTCAAAGTACACATGTTCC
TCCGCTCACGTTCGGTGCTGGGACCAAGCTGGAGCTGAAACGGG
CTGATGCTGCACCAACTGTATCCATCTTCCCAGGCTCGGGCGGT
GGTGGGTCGGGTGGCGAGGTGAAGCTTCAGCAGTCTGGACCTAG
CCTGGTGGAGCCTGGCGCTTCAGTGATGATATCCTGCAAGGCTT
CTGGTTCCTCATTCACTGGCTACAACATGAACTGGGTGAGGCAG
AACATTGGAAAGAGCCTTGAATGGATTGGAGCTATTGATCCTTA
CTATGGTGGAACTAGCTACAACCAGAAGTTCAAGGGCAGGGCCA
CATTGACTGTAGACAAATCGTCCAGCACAGCCTACATGCACCTC
AAGAGCCTGACATCTGAGGACTCTGCAGTCTATTACTGTGTAAG
CGGAATGGAGTACTGGGGTCAAGGAACCTCAGTCACCGTCTCCT
CAGCCAAAACGACACCCCCATCAGTCTATGGAAGGGTCACCGTC
TCTTCAGCGGAGCCCAAATCTTGTGACAAAACTCACACATGCCC
ACCGTGCCCGGATCCCAAATTTTGGGTGCTGGTGGTGGTTGGTG
GAGTCCTGGCTTGCTATAGCTTGCTAGTAACAGTGGCCTTTATT
ATTTTCTGGGTGAGGAGTAAGAGGAGCAGGCTCCTGCACAGTGA
CTACATGAACATGACTCCCCGCCGCCCCGGGCCCACCCGCAAGC
ATTACCAGCCCTATGCCCCACCACGCGACTTCGCAGCCTATCGC
TCCAAACGGGGCAGAAAGAAACTCCTGTATATATTCAAACAACC
ATTTATGAGACCAGTACAAACTACTCAAGAGGAAGATGGCTGTA
GCTGCCGATTTCCAGAAGAAGAAGAAGGAGGATGTGAACTGAGA
GTGAAGTTCAGCAGGAGCGCAGACGCCCCCGCGTACCAGCAGGG
CCAGAACCAGCTCTATAACGAGCTCAATCTAGGACGAAGAGAGG
AGTACGATGTTTTGGACAAGAGACGTGGCCGGGACCCTGAGATG
GGGGGAAAGCCGAGAAGGAAGAACCCTCAGGAAGGCCTGTACAA
TGAACTGCAGAAAGATAAGATGGCGGAGGCCTACAGTGAGATTG
GGATGAAAGGCGAGCGCCGGAGGGGCAAGGGGCACGATGGCCTT
TACCAGGGTCTCAGTACAGCCACCAAGGACACCTACGACGCCCT
TCACATGCAGGCCCTGCCCCCTCGCTAA.

Namely, the nucleotide sequence SEQ ID NO: 50 comprises the following sequences:

iCasp9 sequence—the inducible caspase-9 expression cassette, encoded by the nucleotide sequence:

(SEQ ID NO: 33)
ATGCTCGAGGGAGTGCAGGTGGAGACTATCTCCCCAGGAGACGG
GCGCACCTTCCCCAAGCGCGGCCAGACCTGCGTGGTGCACTACA
CCGGGATGCTTGAAGATGGAAAGAAAGTTGATTCCTCCCGGGAC
AGAAACAAGCCCTTTAAGTTTATGCTAGGCAAGCAGGAGGTGAT
CCGAGGCTGGGAAGAAGGGGTTGCCCAGATGAGTGTGGGTCAGA
GAGCCAAACTGACTATATCTCCAGATTATGCCTATGGTGCCACT
GGGCACCCAGGCATCATCCCACCACATGCCACTCTCGTCTTCGA
TGTGGAGCTTCTAAAACTGGAATCTGGCGGTGGATCCGGAGTCG
ACGGATTTGGTGATGTCGGTGCTCTTGAGAGTTTGAGGGGAAAT
GCAGATTTGGCTTACATCCTGAGCATGGAGCCCTGTGGCCACTG
CCTCATTATCAACAATGTGAACTTCTGCCGTGAGTCCGGGCTCC
GCACCCGCACTGGCTCCAACATCGACTGTGAGAAGTTGCGGCGT
CGCTTCTCCTCGCTGCATTTCATGGTGGAGGTGAAGGGCGACCT
GACTGCCAAGAAAATGGTGCTGGCTTTGCTGGAGCTGGCGCgGC
AGGACCACGGTGCTCTGGACTGCTGCGTGGTGGTCATTCTCTCT
CACGGCTGTCAGGCCAGCCACCTGCAGTTCCCAGGGGCTGTCTA
CGGCACAGATGGATGCCCTGTGTCGGTCGAGAAGATTGTGAACA
TCTTCAATGGGACCAGCTGCCCCAGCCTGGGAGGGAAGCCCAAG
CTCTTTTTCATCCAGGCCTGTGGTGGGGAGCAGAAAGACCATGG
GTTTGAGGTGGCCTCCACTTCCCCTGAAGACGAGTCCCCTGGCA
GTAACCCCGAGCCAGATGCCACCCCGTTCCAGGAAGGTTTGAGG
ACCTTCGACCAGCTGGACGCCATATCTAGTTTGCCCACACCCAG
TGACATCTTTGTGTCCTACTCTACTTTCCCAGGTTTTGTTTCCT
GGAGGGACCCCAAGAGTGGCTCCTGGTACGTTGAGACCCTGGAC
GACATCTTTGAGCAGTGGGCTCACTCTGAAGACCTGCAGTCCCT
CCTGCTTAGGGTCGCTAATGCTGTTTCGGTGAAAGGGATTTATA
AACAGATGCCTGGTTGCTTTAATTTCCTCCGGAAAAAACTTTTC
TTTAAAACATCAGCTAGC,
which is linked by a linker AGAGCC to
a T2A Peptide (2A) sequence:
(SEQ ID NO: 34)
GAGGGCAGGGGAAGTCTTCTAACATGCGGGGACGTGGAGGAAAA
TCCCGGGCCC,
a Signal peptide:
(SEQ ID NO: 35)
ATGGAGTTTGGGCTGAGCTGGCTTTTTCTTGTGGCTATTTTAAA
AGGTGTCCAGTGCTCTAGA,
Anti GD2(14G2a) VL sequence:
(SEQ ID NO: 36)
GATATTTTGCTGACCCAAACTCCACTCTCCCTGCCTGTCAGTCT
TGGAGATCAAGCCTCCATCTCTTGCAGATCTAGTCAGAGTCTTG
TACACCGTAATGGAAACACCTATTTACATTGGTACCTGCAGAAG
CCAGGCCAGTCTCCAAAGCTCCTGATTCACAAAGTTTCCAACCG
ATTTTCTGGGGTCCCAGACAGGTTCAGTGGCAGTGGATCAGGGA
CAGATTTCACACTCAAGATCAGCAGAGTGGAGGCTGAGGATCTG
GGAGTTTATTTCTGTTCTCAAAGTACACATGTTCCTCCGCTCAC
GTTCGGTGCTGGGACCAAGCTGGAGCTGAAACGGGCTGATGCTG
CACCAACTGTATCCATCTTCCCA,
Anti GD2(14G2a) VH sequence:
(SEQ ID NO: 37)
GAGGTGAAGCTTCAGCAGTCTGGACCTAGCCTGGTGGAGCCTGG
CGCTTCAGTGATGATATCCTGCAAGGCTTCTGGTTCCTCATTCA
CTGGCTACAACATGAACTGGGTGAGGCAGAACATTGGAAAGAGC
CTTGAATGGATTGGAGCTATTGATCCTTACTATGGTGGAACTAG
CTACAACCAGAAGTTCAAGGGCAGGGCCACATTGACTGTAGACA
AATCGTCCAGCACAGCCTACATGCACCTCAAGAGCCTGACATCT
GAGGACTCTGCAGTCTATTACTGTGTAAGCGGAATGGAGTACTG
GGGTCAAGGAACCTCAGTCACCGTCTCCTCA,
The G7S2 linker (which links anti GD2
(14G2a) VL sequence and anti GD2 (14G2a) VH
sequence) sequence:
(SEQ ID NO: 44)
GGCTCGGGCGGTGGTGGGTCGGGTGGC;
IgG-based hinge sequence:
(SEQ ID NO: 45)
GCCAAAACGACACCCCCATCAGTCTATGGAAGGGTCACCGTCTC
TTCAGCGGAGCCCAAATCTTGTGACAAAACTCACACATGCCCAC
CGTGCCCG;
which is linked by the Linker (connection
sequence) GATCCCAAA to a transmembrane
domain
CD28TM sequence:
(SEQ ID NO: 46)
TTTTGGGTGCTGGTGGTGGTTGGTGGAGTCCTGGCTTGCTATAG
CTTGCTAGTAACAGTGGCCTTTATTATTTTCTGGGTG;
CD28 cytoplasmic sequence:
(SEQ ID NO: 47)
AGGAGTAAGAGGAGCAGGCTCCTGCACAGTGACTACATGAACAT
GACTCCCCGCCGCCCCGGGCCCACCCGCAAGCATTACCAGCCCT
ATGCCCCACCACGCGACTTCGCAGCCTATCGCTCC
CD137 (4-1BB) sequence:
(SEQ ID NO: 48)
AAACGGGGCAGAAAGAAACTCCTGTATATATTCAAACAACCATT
TATGAGACCAGTACAAACTACTCAAGAGGAAGATGGCTGTAGCT
GCCGATTTCCAGAAGAAGAAGAAGGAGGATGTGAACTG
CD3-Zeta chain sequence:
(SEQ ID NO: 49)
AGAGTGAAGTTCAGCAGGAGCGCAGACGCCCCCGCGTACCAGCA
GGGCCAGAACCAGCTCTATAACGAGCTCAATCTAGGACGAAGAG
AGGAGTACGATGTTTTGGACAAGAGACGTGGCCGGGACCCTGAG
ATGGGGGGAAAGCCGAGAAGGAAGAACCCTCAGGAAGGCCTGTA
CAATGAACTGCAGAAAGATAAGATGGCGGAGGCCTACAGTGAGA
TTGGGATGAAAGGCGAGCGCCGGAGGGGCAAGGGGCACGATGGC
CTTTACCAGGGTCTCAGTACAGCCACCAAGGACACCTACGACGC
CCTTCACATGCAGGCCCTGCCCCCTCGCTAA.

According to the present invention, the vector comprising the nucleotide sequence defined above can be chosen from a DNA vector, a RNA vector, a plasmid, a lentivirus vector, adenoviral vector, retrovirus vector, such as γ-retroviral vector, or non-viral vector.

The present invention also concerns a cell, such as T cell, such as alfa/beta and gamma/delta T cell, NK cells, NK-T cells as well as macrophages or monocyte cells, comprising the anti-GD2 chimeric antigen receptor as defined above, and/or the vector as defined above, for use in the treatment of solid tumors, wherein said solid tumor is not neuroblastoma, diffuse midline gliomas (DMG) H3K27M-mutant, or osteosarcoma.

In addition, according to an embodiment of the invention, said solid tumor is not high grade glioma. According to a further embodiment of the invention, said solid tumor is not rhabdomyosarcoma or Ewing sarcoma. According to an embodiment of the invention, said solid tumor is not sarcoma.

According to a further embodiment of the present invention, said solid tumor is not medulloblastoma. According to an embodiment of the invention, said tumor is not glioma.

According to the present invention, the solid tumors to be treated are tumors expressing GD2 or tumors expressing GD2 after a treatment with a compound able to enhance the expression of GD2, such as an Enhancer of Zeste Homolog 1 or 2 (EZH2) inhibitor.

As stated above, said solid tumors can be chosen from brain tumors, excluding diffuse midline gliomas (DMG) H3K27M-mutant, such as diffuse intrinsic pontine gliomas (DIPG) different from diffuse midline glioma (DMG) H3K27M-mutant, adult-type diffuse gliomas, pediatric-type diffuse low-grade gliomas, pediatric-type diffuse high-grade gliomas, astrocytic gliomas, medulloblastoma, for example SHH, G3, G4 and WNT medulloblastoma subgroups, glioneuronal and neuronal tumors, ependymal tumors, choroid plexus tumors, embryonal tumors, pineal tumors, cranial and paraspinal nerve tumors; extra-cranic tumors, such as sarcoma different from osteosarcoma, for example rhabdomyosarcoma, in particular Alveolar Rhabdomyosarcoma or embryonal Rhabdomyosarcoma, Ewing's sarcoma (EWS), desmoplastic small round cell tumors, leiomyosarcomas and liposarcomas; retinoblastoma; melanoma; lung cancer; gastric cancer; bladder cancer, head and neck cancer; ovarian cancer; breast cancer; pancreatic cancer; colorectal cancer or other tumors expressing GD2.

According to the present invention, said tumor can be a tumor having an expression of GD2 higher than the expression of GD2 in a negative healthy control, namely healthy peripheral blood mononuclear cells (PBMC), wherein the expression of GD2 is measured as the percentage of GD2+ cells with respect to the total number of cells of the tumor or of the healthy peripheral blood mononuclear cells (the percentage of GD2 positive cells in healthy PBMC being equal to 0.43%+0.90%). Preferably, said tumor is a tumor having an expression of GD2 higher than 2%, i.e. having a percentage of GD2+ cells higher than 2%.

According to an embodiment of the invention, said brain tumor is not high grade glioma. According to a further embodiment of the invention, said sarcoma is not rhabdomyosarcoma or Ewing sarcoma. According to an embodiment of the invention, said solid tumor is not sarcoma. According to a further embodiment of the present invention, said brain tumor is not medulloblastoma.

According to an embodiment of the present invention, said cell can further comprise a suicide gene inducible amino acid sequence such as a chimeric Caspase-9 polypeptide or a herpes simplex virus thymidine kinase (HSV-TK) or ACD19 sequence as a safety switch.

In particular, the chimeric Caspase-9 polypeptide can comprise or consist of:

iCasp9 cassette:
(SEQ ID NO: 1)
MLEGVQVETISPGDGRTFPKRGQTCVVHYTGMLEDGKKVDSSRD
RNKPFKFMLGKQEVIRGWEEGVAQMSVGQRAKLTISPDYAYGAT
GHPGIIPPHATLVFDVELLKLESGGGSGVDGFGDVGALESLRGN
ADLAYILSMEPCGHCLIINNVNFCRESGLRTRTGSNIDCEKLRR
RFSSLHFMVEVKGDLTAKKMVLALLELARQDHGALDCCVVVILS
HGCQASHLQFPGAVYGTDGCPVSVEKIVNIFNGTSCPSLGGKPK
LFFIQACGGEQKDHGFEVASTSPEDESPGSNPEPDATPFQEGLR
TFDQLDAISSLPTPSDIFVSYSTFPGFVSWRDPKSGSWYVETLD
DIFEQWAHSEDLQSLLLRVANAVSVKGIYKQMPGCFNFLRKKLF
FKTSAS,
which is linked by a linker, such as RA, to:
a T2A peptide (2A):
(SEQ ID NO: 2)
EGRGSLLTCGDVEENPGP.

According to the present invention, said cell can be obtained in culture conditions wherein both or each of IL-7 and/or IL-15 are present, for example in the culture conditions of the activation step, transduction step and/or expansion step of the process for the preparation of said cell.

It is a further object of the present invention a pharmaceutical composition comprising the nucleotide sequence as defined above, or the vector as defined above, or the cell as defined above with one or more pharmaceutically acceptable excipients and/or adjuvants, for use in the treatment of solid tumors, wherein said solid tumor is not neuroblastoma, diffuse midline gliomas (DMG) H3K27M-mutant or osteosarcoma.

According to the present invention, the solid tumors to be treated are tumors expressing GD2 or tumors expressing GD2 after a treatment with a compound able to enhance the expression of GD2, such as an Enhancer of Zeste Homolog 1 or 2 (EZH2) inhibitor.

In addition, according to an embodiment of the invention, said solid tumor is not high grade glioma. According to a further embodiment of the invention, said solid tumor is not rhabdomyosarcoma or Ewing sarcoma. According to an embodiment of the invention, said solid tumor is not sarcoma.

According to a further embodiment of the present invention, said solid tumor is not medulloblastoma.

As stated above, according to the present invention, said solid tumors can be chosen from brain tumors, excluding diffuse midline gliomas (DMG) H3K27M-mutant, such as diffuse intrinsic pontine gliomas (DIPG) different from diffuse midline glioma (DMG) H3K27M-mutant, adult-type diffuse gliomas, pediatric-type diffuse low-grade gliomas, pediatric-type diffuse high-grade gliomas, astrocytic gliomas, medulloblastoma, for example SHH, G3, G4 and WNT medulloblastoma subgroups, glioneuronal and neuronal tumors, ependymal tumors, choroid plexus tumors, embryonal tumors, pineal tumors, cranial and paraspinal nerve tumors; extra-cranic tumors, such as sarcoma different from osteosarcoma, for example rhabdomyosarcoma, in particular Alveolar Rhabdomyosarcoma or embryonal Rhabdomyosarcoma, Ewing's sarcoma (EWS), desmoplastic small round cell tumors, leiomyosarcomas and liposarcomas; retinoblastoma; melanoma; lung cancer; gastric cancer; bladder cancer, head and neck cancer; ovarian cancer; breast cancer; pancreatic cancer; colorectal cancer or other tumors expressing GD2.

According to an embodiment of the invention, said brain tumor is not high grade glioma. According to a further embodiment of the invention, said sarcoma is not rhabdomyosarcoma or Ewing sarcoma. According to an embodiment of the invention, said solid tumor is not sarcoma. According to a further embodiment of the present invention, said brain tumor is not medulloblastoma.

According to the present invention, said tumor can be a tumor having an expression of GD2 higher than the expression of GD2 in a negative healthy control, namely healthy peripheral blood mononuclear cells (PBMC), wherein the expression of GD2 is measured as the percentage of GD2+ cells with respect to the total number of cells of the tumor or of the healthy peripheral blood mononuclear cells (the percentage of GD2 positive cells in healthy PBMC being equal to 0.43%+0.90%). Preferably, said tumor is a tumor having an expression of GD2 higher than 2%, i.e. having a percentage of GD2+ cells higher than 2%.

It is a further object of the present invention a combination of
an anti-GD2 chimeric antigen receptor as defined above, or of a nucleotide sequence as defined above, or of a vector as defined above, or of a cell as defined above or of a pharmaceutical composition as defined above, with
an Enhancer of Zeste Homolog 1 or 2 (EZH2) inhibitor or with a pharmaceutical composition comprising an Enhancer of Zeste Homolog 1 or 2 (EZH2) inhibitor and one or more excipients and/or adjuvants,
for separate or sequential use in the treatment of tumors solid tumors.

According to the present invention, the solid tumors to be treated are tumors expressing GD2 or tumors able to express GD2 after a treatment with a Enhancer of Zeste Homolog 1 or 2 (EZH2) inhibitor.

In particular, the combination of the present invention can be advantageously used when the tumor does not express GD2 or has a low expression of GD2. In fact, according to the present invention, the treatment with a EZH2 inhibitor increases the expression of GD2 by the tumor cells and, therefore, the successive administration of the anti-GD2 CAR according to the invention becomes effective against the tumor. More in detail, the EZH2 inhibitor can be administered from 3 to 21 days, preferably from three to seven days, before the administration of the anti-GD2 CAR.

According to the present invention, “Separate use” is understood as meaning the administration, at the same time, of the two compounds of the combination according to the invention in distinct pharmaceutical forms.

“Sequential use” is understood as meaning the successive administration of the two compounds of the combination according to the invention, each in a distinct pharmaceutical form.

According to the combination of the present invention for use according to the invention, said solid tumors can be chosen from brain tumors, such as glioblastoma, such as glioma, for example diffuse intrinsic pontine glioma (DIPG), including diffuse midline glioma (DMG) H3K27M-mutant, high grade glioma, adult-type diffuse gliomas, pediatric-type diffuse low-grade gliomas, pediatric-type diffuse high-grade gliomas astrocytic glioma, medulloblastoma, for example SHH, G3, G4 and WNT medulloblastoma subgroups, glioneuronal and neuronal tumors, ependymal tumors, choroid plexus tumors, embryonal tumors, pineal tumors, cranial and paraspinal nerve tumors; extra-cranic tumors, such as sarcoma, in particular sarcoma with low expression of GD2, for example osteosarcoma, rhabdomyosarcoma, in particular Alveolar Rhabdomyosarcoma, or embryonal Rhabdomyosarcoma, Ewing's sarcoma (EWS), desmoplastic small round cell tumors, leiomyosarcomas and liposarcomas; retinoblastoma; melanoma; lung cancer; breast cancer; bladder cancer; gastric cancer; head and neck cancer; ovarian cancer; neuroblastoma pancreatic cancer; colorectal cancer.

According to the present invention, said Enhancer of Zeste Homolog 2 inhibitor can be chosen from the group consisting of Tazemetostat, CPI-1205, which is an orally bioavailable, indole-based, small-molecule inhibitor of EZH268 69; PF-06821497⁷⁰, GSK126 (GSK2816126)⁷¹, preferably Tazemetostat and/or EZH1/2 dual inhibitor such as Valemetostat.

According to an embodiment of the present invention, said Enhancer of Zeste Homolog 1 or 2 inhibitor can be administered from a minimum of three days to a maximum of 21 days, preferably from three to seven days, before said anti-GD2 chimeric antigen receptor, nucleotide sequence, vector, cell or pharmaceutical composition.

The present invention also concerns a kit of parts comprising or consisting of:
A) an anti-GD2 chimeric antigen receptor as defined in any one of claims 1-14, or a nucleotide sequence or a vector according to any one of claims 15-23, or a cell according to any one of claims 24-28 or a pharmaceutical composition according to any one of claims 29-30; and
B) an enhancer of Zeste Homolog 1 or 2 (EZH2) inhibitor or a pharmaceutical composition comprising an Enhancer of Zeste Homolog 1 or 2 (EZH2) inhibitor and one or more excipients and/or adjuvants,
wherein A) and B) are to administered separately or sequentially.

According to the invention, said Enhancer of Zeste Homolog 2 inhibitor can be chosen from the group consisting of Tazemetostat, CPI-1205, which is an orally bioavailable, indole-based, small-molecule inhibitor of EZH268 69; PF-0682149770 GSK126 (GSK2816126) 71, preferably Tazemetostat.

A further object of the present invention is anti-GD2 chimeric antigen receptor comprising or consisting of, from the N-terminus to the C-terminus:
a) a signal peptide,
b) an anti GD2 single chain antibody domain,
c) a hinge,
d) a trans membrane domain,
e) at least two co-stimulatory signaling domains, and
f) CD3Zeta chain sequence,
wherein said anti GD2 single chain antibody domain comprises or consists of anti GD2 VL sequence and anti GD2 VH sequence linked each other by a linker, said linker being a short flexible glycines-rich linker with a length from 7 to 14 amino acids, such as from 7 to 12, from 7 to 10 or 8 amino acids, for example said linker being selected from G7S2 linker GSGGGGSGG (SEQ ID NO: 13), (G4S) 2 linker GGGGSGGGG (SEQ ID NO: 14), G4SG2 linker GGGGSGG (SEQ ID NO: 15), G3SG4 linker GGGSGGGG (SEQ ID NO: 16) SG4SG3 linker SGGGGSGGG (SEQ ID NO: 17), (SG4) 2 S linker SGGGGSGGGGS (SEQ ID NO: 18), (SG4) 2 SG linker SGGGGSGGGGSG (SEQ ID NO: 19), (SG4) 2 SG3 linker SGGGGGGGGSGGG linker (SEQ ID NO: 20), (SG4) 2 SGGGGSGGGG (SEQ ID NO: 21), or (SG4) 2 SG2 SGGGGSGGGGSGG (SEQ ID NO: 22), preferably G7S2 linker GSGGGGSGG (SEQ ID NO: 13).

According to the above anti-GD2 chimeric antigen receptor the anti GD2 VL sequence can comprise CDR1 sequence QSLVHRNGNTY (SEQ ID NO: 5), CDR2 sequence: KVS and CDR3 sequence SQSTHVP (SEQ ID NO: 7); whereas
anti GD2 VH sequence can comprise CDR1 sequence: GSSFTGYN (SEQ ID NO: 8), CDR2 sequence: IDPYYGGT (SEQ ID NO: 9) and CDR3 sequence: VSGMEY (SEQ ID NO: 10).

According to an embodiment of the present invention, anti GD2 VL sequence can comprise or consist of:

(SEQ ID NO: 11)
DILLTQTPLSLPVSLGDQASISCRSSQSLVHRNGNTYLHWYLQK
PGQSPKLLIHKVSNRFSGVPDRFSGSGSGTDFTLKISRVEAEDL
GVYFCSQSTHVPPLTFGAGTKLELKRADAAPTVSIFP,
and
anti GD2 VH sequence can comprise or
consist of:
(SEQ ID NO: 12)
EVKLQQSGPSLVEPGASVMISCKASGSSFTGYNMNWVRQNIGKS
LEWIGAIDPYYGGTSYNQKFKGRATLTVDKSSSTAYMHLKSLTS
EDSAVYYCVSGMEYWGQGTSVTVSS.

Regarding the hinge of the anti-GD2 chimeric antigen receptor according to the present invention, said hinge can comprise or consist of one or more of the following hinges:

IgG-based hinge of sequence
(SEQ ID NO: 23)
AKTTPPSVYGRVTVSSAEPKSCDKTHTCPPCP;
hinge Spacer-CD8α of sequence
(SEQ ID NO: 24)
PAPRPPTPAPTIASQPLSLRPEACRPAAGGAVHTRGLDFA;
CD8stalk of sequence
(SEQ ID NO: 25)
TTTPAPRPPTPAPTIASQPLSLRPEACRPAAGGAVHTRGLDFACD;
hinge CD28 of sequence
(SEQ ID NO: 26)
EVMYPPPYLDNEKSNGTIIHVKGKHLCPSPLFPGPSKP;
hinge CH2-CH3 of sequence
(SEQ ID NO: 27)
ESKYGPPCPSCPAPEFLGGPSVFLFPPKPKDTLMISRTPEVTCVV
VDVSQEDPEVQFNWYVDGVEVHNAKTKPREEQFNSTYRVVSVLTV
LHQDWLNGKEYKCKVSNKGLPSSIEKTISKAKGQPREPQVYTLPP
SQEEMTKNQVSLTCLVKGFYPSDIAVEWESNGQPENNYKTTPPVL
DSDGSFFLYSRLTVDKSRWQEGNVFSCSVMHEALHNHYTQKSLSL
SLGK;
hinge CH3 of sequence
(SEQ ID NO: 28)
ESKYGPPCPSCPGQPREPQVYTLPPSQEEMTKNQVSLTCLVKGFY
PSDIAVEWESNGQPENNYKTTPPVLDSDGSFFLYSRLTVDKSRWQ
EGNVFSCSVMHEALHNHYTQKSLSLSLGK;

preferably IgG-based hinge of sequence SEQ ID NO: 23.

According the anti-GD2 chimeric antigen receptor of the present invention, said trans membrane domain can be chosen from the group consisting of CD8aTM:

(SEQ ID NO: 6)
CDIYIWAPLAGTCGVLLLSLVIT;
CD28TM:
(SEQ ID NO: 29)
FWVLVVVGGVLACYSLLVTVAFIIFWV;
preferably is
CD28TM of sequence
(SEQ ID NO: 29)
FWVLVVVGGVLACYSLLVTVAFIIFWV.

According to the anti-GD2 chimeric antigen receptor of the invention, said at least two co-stimulatory signaling domains can be chosen from the group consisting of:
a sequence obtained by linking:

CD28 cytoplasmic sequence:
(SEQ ID NO: 30)
RSKRSRLLHSDYMNMTPRRPGPTRKHYQPYAPPRDFAAYRS
to
OX40 sequence:
(SEQ ID NO: 32)
RDQRLPPDAHKPPGGGSFRTPIQEEQADAHSTLAKI,

wherein CD28 cytoplasmic sequence is positioned before or after OX40 sequence;
a sequence obtained by linking:
CD28 cytoplasmic sequence:
RSKRSRLLHSDYMNMTPRRPGPTRKHYQPYAPPRDFAAYRS (SEQ ID NO: 30) to
CD137 (4-1BB) sequence:
KRGRKKLLYIFKQPFMRPVQTTQEEDGCSCRFPEEEEGGCEL (SEQ ID NO: 31), wherein CD28 cytoplasmic sequence is positioned before or after CD137 (4-1BB) sequence;
a sequence obtained by linking:
OX40 sequence:
RDQRLPPDAHKPPGGGSFRTPIQEEQADAHSTLAKI (SEQ ID NO: 32) to CD28 cytoplasmic sequence:
RSKRSRLLHSDYMNMTPRRPGPTRKHYQPYAPPRDFAAYRS (SEQ ID NO: 30), wherein OX40 sequence is positioned before or after CD28 cytoplasmic sequence; or
a sequence obtained by linking:
OX40 sequence:
RDQRLPPDAHKPPGGGSFRTPIQEEQADAHSTLAKI (SEQ ID NO: 32) to CD137 (4-1BB) sequence:
KRGRKKLLYIFKQPFMRPVQTTQEEDGCSCRFPEEEEGGCEL (SEQ ID NO: 31), wherein OX40 sequence is positioned before or after CD137 (4-1BB) sequence;
preferably a sequence obtained by linking:
CD28 cytoplasmic sequence:
RSKRSRLLHSDYMNMTPRRPGPTRKHYQPYAPPRDFAAYRS (SEQ ID NO: 30), to
CD137 (4-1BB) sequence:
KRGRKKLLYIFKQPFMRPVQTTQEEDGCSCRFPEEEEGGCEL (SEQ ID NO: 31), wherein CD28 cytoplasmic sequence is positioned before CD137 (4-1BB) sequence.

According to the present invention, CD3-Zeta chain sequence can be RVKFSRSADAPAYQQGQNQLYNELNLGRREEYDVLDKRRGRDPEMGGKPRRK NPQEGLYNELQKDKMAEAYSEIGMKGERRRGKGHDGLYQGLSTATKDTYDALH MQALPPR* (SEQ ID NO: 53).

In addition, the anti-GD2 chimeric antigen receptor according to the present invention can further comprise cytoplasmic moiety of CD8cyt of sequence LYCNHRN (SEQ ID NO: 51) between the trans membrane domain and the co-stimulatory signaling domain.

According to the anti-GD2 chimeric antigen receptor of the present invention, said signal peptide can comprise or consist of MEFGLSWLFLVAILKGVQCSR (SEQ ID NO: 3).

According to an embodiment of the present invention, the anti-GD2 chimeric antigen receptor comprises or consists of the following sequence:

GD2.CAR-28.4-1BB.ζ
(SEQ ID NO: 39)
MEFGLSWLFLVAILKGVQCSRDILLTQTPLSLPVSLGDQASISC
RSSQSLVHRNGNTYLHWYLQKPGQSPKLLIHKVSNRFSGVPDRF
SGSGSGTDFTLKISRVEAEDLGVYFCSQSTHVPPLTFGAGTKLE
LKRADAAPTVSIFPGSGGGGSGGEVKLQQSGPSLVEPGASVMIS
CKASGSSFTGYNMNWVRQNIGKSLEWIGAIDPYYGGTSYNQKFK
GRATLTVDKSSSTAYMHLKSLTSEDSAVYYCVSGMEYWGQGTSV
TVSSAKTTPPSVYGRVTVSSAEPKSCDKTHTCPPCPDPKFWVLV
VVGGVLACYSLLVTVAFIIFWVRSKRSRLLHSDYMNMTPRRPGP
TRKHYQPYAPPRDFAAYRSKRGRKKLLYIFKQPFMRPVQTTQEE
DGCSCRFPEEEEGGCELRVKFSRSADAPAYQQGQNQLYNELNLG
RREEYDVLDKRRGRDPEMGGKPRRKNPQEGLYNELQKDKMAEAY
SEIGMKGERRRGKGHDGLYQGLSTATKDTYDALHMQALPPR*.

In particular, sequence SEQ ID NO: 39 comprises

a Signal peptide:
(SEQ ID NO: 3)
MEFGLSWLFLVAILKGVQCSR;
anti GD2(14G2a) VL sequence:
(SEQ ID NO: 11)
DILLTQTPLSLPVSLGDQASISCRSSQSLVHRNGNTYLHWYLQKP
GQSPKLLIHKVSNRFSGVPDRFSGSGSGTDFTLKISRVEAEDLGV
YFCSQSTHVPPLTFGAGTKLELKRADAAPTVSIFP.
G7S2 linker:
(SEQ ID NO: 13)
GSGGGGSGG
anti GD2(14G2a) VH sequence:
(SEQ ID NO: 12)
EVKLQQSGPSLVEPGASVMISCKASGSSFTGYNMNWVRQNIGKSL
EWIGAIDPYYGGTSYNQKFKGRATLTVDKSSSTAYMHLKSLTSED
SAVYYCVSGMEYWGQGTSVTVSS.
IgG-based hinge:
(SEQ ID NO: 23)
AKTTPPSVYGRVTVSSAEPKSCDKTHTCPPCP
which is linked by the Linker (connection
sequence) DPK to a transmembrane domain:
CD28TM:
(SEQ ID NO: 29)
FWVLVVVGGVLACYSLLVTVAFIIFWV
CD28 cytoplasmic sequence:
(SEQ ID NO: 30)
RSKRSRLLHSDYMNMTPRRPGPTRKHYQPYAPPRDFAAYRS
CD137 (4-1BB) sequence:
(SEQ ID NO: 31)
KRGRKKLLYIFKQPFMRPVQTTQEEDGCSCRFPEEEEGGCEL
CD3-Zeta chain:
(SEQ ID NO: 53)
RVKFSRSADAPAYQQGQNQLYNELNLGRREEYDVLDKRRGRDPEM
GGKPRRKNPQEGLYNELQKDKMAEAYSEIGMKGERRRGKGHDGLY
QGLSTATKDTYDALHMQALPPR*.

The present invention concerns also a nucleotide sequence or a vector comprising the nucleotide sequence, said nucleotide sequence comprising or consisting of a nucleotide sequence, which encodes the anti-GD2 chimeric antigen receptor as defined above.

In particular, the present invention concerns the nucleotide sequence or the vector according to the above, wherein

anti GD2 VL sequence can be encoded by the
nucleotide sequence
(SEQ ID NO: 36)
GATATTTTGCTGACCCAAACTCCACTCTCCCTGCCTGTCAGTCTT
GGAGATCAAGCCTCCATCTCTTGCAGATCTAGTCAGAGTCTTGTA
CACCGTAATGGAAACACCTATTTACATTGGTACCTGCAGAAGCCA
GGCCAGTCTCCAAAGCTCCTGATTCACAAAGTTTCCAACCGATTT
TCTGGGGTCCCAGACAGGTTCAGTGGCAGTGGATCAGGGACAGAT
TTCACACTCAAGATCAGCAGAGTGGAGGCTGAGGATCTGGGAGTT
TATTTCTGTTCTCAAAGTACACATGTTCCTCCGCTCACGTTCGGT
GCTGGGACCAAGCTGGAGCTGAAACGGGCTGATGCTGCACCAACT
GTATCCATCTTCCCA,
and
anti GD2 VH sequence can be encoded by the
nucleotide sequence
(SEQ ID NO: 37)
GAGGTGAAGCTTCAGCAGTCTGGACCTAGCCTGGTGGAGCCTGGC
GCTTCAGTGATGATATCCTGCAAGGCTTCTGGTTCCTCATTCACT
GGCTACAACATGAACTGGGTGAGGCAGAACATTGGAAAGAGCCTT
GAATGGATTGGAGCTATTGATCCTTACTATGGTGGAACTAGCTAC
AACCAGAAGTTCAAGGGCAGGGCCACATTGACTGTAGACAAATCG
TCCAGCACAGCCTACATGCACCTCAAGAGCCTGACATCTGAGGAC
TCTGCAGTCTATTACTGTGTAAGCGGAATGGAGTACTGGGGTCAA
GGAACCTCAGTCACCGTCTCCTCA.

In particular, said nucleotide sequence can be

GD2.CAR-28.4-1BB.ζ
(SEQ ID NO: 52)
ATGGAGTTTGGGCTGAGCTGGCTTTTTCTTGTGGCTATTTTAAAA
GGTGTCCAGTGCTCTAGAGATATTTTGCTGACCCAAACTCCACTC
TCCCTGCCTGTCAGTCTTGGAGATCAAGCCTCCATCTCTTGCAGA
TCTAGTCAGAGTCTTGTACACCGTAATGGAAACACCTATTTACAT
TGGTACCTGCAGAAGCCAGGCCAGTCTCCAAAGCTCCTGATTCAC
AAAGTTTCCAACCGATTTTCTGGGGTCCCAGACAGGTTCAGTGGC
AGTGGATCAGGGACAGATTTCACACTCAAGATCAGCAGAGTGGAG
GCTGAGGATCTGGGAGTTTATTTCTGTTCTCAAAGTACACATGTT
CCTCCGCTCACGTTCGGTGCTGGGACCAAGCTGGAGCTGAAACGG
GCTGATGCTGCACCAACTGTATCCATCTTCCCAGGCTCGGGCGGT
GGTGGGTCGGGTGGCGAGGTGAAGCTTCAGCAGTCTGGACCTAGC
CTGGTGGAGCCTGGCGCTTCAGTGATGATATCCTGCAAGGCTTCT
GGTTCCTCATTCACTGGCTACAACATGAACTGGGTGAGGCAGAAC
ATTGGAAAGAGCCTTGAATGGATTGGAGCTATTGATCCTTACTAT
GGTGGAACTAGCTACAACCAGAAGTTCAAGGGCAGGGCCACATTG
ACTGTAGACAAATCGTCCAGCACAGCCTACATGCACCTCAAGAGC
CTGACATCTGAGGACTCTGCAGTCTATTACTGTGTAAGCGGAATG
GAGTACTGGGGTCAAGGAACCTCAGTCACCGTCTCCTCAGCCAAA
ACGACACCCCCATCAGTCTATGGAAGGGTCACCGTCTCTTCAGCG
GAGCCCAAATCTTGTGACAAAACTCACACATGCCCACCGTGCCCG
GATCCCAAATTTTGGGTGCTGGTGGTGGTTGGTGGAGTCCTGGCT
TGCTATAGCTTGCTAGTAACAGTGGCCTTTATTATTTTCTGGGTG
AGGAGTAAGAGGAGCAGGCTCCTGCACAGTGACTACATGAACATG
ACTCCCCGCCGCCCCGGGCCCACCCGCAAGCATTACCAGCCCTAT
GCCCCACCACGCGACTTCGCAGCCTATCGCTCCAAACGGGGCAGA
AAGAAACTCCTGTATATATTCAAACAACCATTTATGAGACCAGTA
CAAACTACTCAAGAGGAAGATGGCTGTAGCTGCCGATTTCCAGAA
GAAGAAGAAGGAGGATGTGAACTGAGAGTGAAGTTCAGCAGGAGC
GCAGACGCCCCCGCGTACCAGCAGGGCCAGAACCAGCTCTATAAC
GAGCTCAATCTAGGACGAAGAGAGGAGTACGATGTTTTGGACAAG
AGACGTGGCCGGGACCCTGAGATGGGGGGAAAGCCGAGAAGGAAG
AACCCTCAGGAAGGCCTGTACAATGAACTGCAGAAAGATAAGATG
GCGGAGGCCTACAGTGAGATTGGGATGAAAGGCGAGCGCCGGAGG
GGCAAGGGGCACGATGGCCTTTACCAGGGTCTCAGTACAGCCACC
AAGGACACCTACGACGCCCTTCACATGCAGGCCCTGCCCCCTCGC
TAA.

More in particular, the sequence SEQ ID NO: 52 comprises:

Signal peptide:
(SEQ ID NO: 35)
ATGGAGTTTGGGCTGAGCTGGCTTTTTCTTGTGGCTATTTTAAAA
GGTGTCCAGTGCTCTAGA,
The G7S2 linker (which links anti GD2 (14G2a)
VL sequence and anti GD2 (14G2a) VH sequence)
sequence:
(SEQ ID NO: 44)
GGCTCGGGCGGTGGTGGGTCGGGTGGC;
IgG-based hinge sequence:
(SEQ ID NO: 45)
GCCAAAACGACACCCCCATCAGTCTATGGAAGGGTCACCGTCTCT
TCAGCGGAGCCCAAATCTTGTGACAAAACTCACACATGCCCACCG
TGCCCG;
which is linked by the Linker (connection
sequence) GATCCCAAA to a transmembrane domain
CD28TM sequence:
(SEQ ID NO: 46)
TTTTGGGTGCTGGTGGTGGTTGGTGGAGTCCTGGCTTGCTATAGC
TTGCTAGTAACAGTGGCCTTTATTATTTTCTGGGTG;
CD28 cytoplasmic sequence:
(SEQ ID NO: 47)
AGGAGTAAGAGGAGCAGGCTCCTGCACAGTGACTACATGAACATG
ACTCCCCGCCGCCCCGGGCCCACCCGCAAGCATTACCAGCCCTAT
GCCCCACCACGCGACTTCGCAGCCTATCGCTCC
CD137 (4-1BB) sequence:
(SEQ ID NO: 48)
AAACGGGGCAGAAAGAAACTCCTGTATATATTCAAACAACCATTT
ATGAGACCAGTACAAACTACTCAAGAGGAAGATGGCTGTAGCTGC
CGATTTCCAGAAGAAGAAGAAGGAGGATGTGAACTG
CD3-Zeta chain sequence:
(SEQ ID NO: 49)
AGAGTGAAGTTCAGCAGGAGCGCAGACGCCCCCGCGTACCAGCAG
GGCCAGAACCAGCTCTATAACGAGCTCAATCTAGGACGAAGAGAG
GAGTACGATGTTTTGGACAAGAGACGTGGCCGGGACCCTGAGATG
GGGGGAAAGCCGAGAAGGAAGAACCCTCAGGAAGGCCTGTACAAT
GAACTGCAGAAAGATAAGATGGCGGAGGCCTACAGTGAGATTGGG
ATGAAAGGCGAGCGCCGGAGGGGCAAGGGGCACGATGGCCTTTAC
CAGGGTCTCAGTACAGCCACCAAGGACACCTACGACGCCCTTCAC
ATGCAGGCCCTGCCCCCTCGCTAA.

According to the present invention, said nucleotide sequence or vector can further comprise a nucleotide sequence encoding a suicide gene inducible amino acid sequence linked to the nucleotide sequence encoding said chimeric antigen receptor by a nucleotide sequence encoding a 2A self-cleaving peptide. In particular, the suicide gene inducible amino acid sequence can be a chimeric Caspase-9 polypeptide or comprises a herpes simplex virus thymidine kinase or ΔCD19 sequence as a safety switch.

Therefore, in the cell, the polynucleotide 2A selfcleaving peptide cuts the peptide comprising the suicide gene inducible amino acid sequence and the chimeric antigen receptor in two separate peptides, i.e., the suicide gene inducible and the chimeric antigen receptor amino acid sequences.

Therefore, the nucleotide sequence according to the present invention can be

iC9.2A.GD2.CAR-28.4-1BB.ζ
(SEQ ID NO: 50)
ATGCTCGAGGGAGTGCAGGTGGAGACTATCTCCCCAGGAGACGGG
CGCACCTTCCCCAAGCGCGGCCAGACCTGCGTGGTGCACTACACC
GGGATGCTTGAAGATGGAAAGAAAGTTGATTCCTCCCGGGACAGA
AACAAGCCCTTTAAGTTTATGCTAGGCAAGCAGGAGGTGATCCGA
GGCTGGGAAGAAGGGGTTGCCCAGATGAGTGTGGGTCAGAGAGCC
AAACTGACTATATCTCCAGATTATGCCTATGGTGCCACTGGGCAC
CCAGGCATCATCCCACCACATGCCACTCTCGTCTTCGATGTGGAG
CTTCTAAAACTGGAATCTGGCGGTGGATCCGGAGTCGACGGATTT
GGTGATGTCGGTGCTCTTGAGAGTTTGAGGGGAAATGCAGATTTG
GCTTACATCCTGAGCATGGAGCCCTGTGGCCACTGCCTCATTATC
AACAATGTGAACTTCTGCCGTGAGTCCGGGCTCCGCACCCGCACT
GGCTCCAACATCGACTGTGAGAAGTTGCGGCGTCGCTTCTCCTCG
CTGCATTTCATGGTGGAGGTGAAGGGCGACCTGACTGCCAAGAAA
ATGGTGCTGGCTTTGCTGGAGCTGGCGCgGCAGGACCACGGTGCT
CTGGACTGCTGCGTGGTGGTCATTCTCTCTCACGGCTGTCAGGCC
AGCCACCTGCAGTTCCCAGGGGCTGTCTACGGCACAGATGGATGC
CCTGTGTCGGTCGAGAAGATTGTGAACATCTTCAATGGGACCAGC
TGCCCCAGCCTGGGAGGGAAGCCCAAGCTCTTTTTCATCCAGGCC
TGTGGTGGGGAGCAGAAAGACCATGGGTTTGAGGTGGCCTCCACT
TCCCCTGAAGACGAGTCCCCTGGCAGTAACCCCGAGCCAGATGCC
ACCCCGTTCCAGGAAGGTTTGAGGACCTTCGACCAGCTGGACGCC
ATATCTAGTTTGCCCACACCCAGTGACATCTTTGTGTCCTACTCT
ACTTTCCCAGGTTTTGTTTCCTGGAGGGACCCCAAGAGTGGCTCC
TGGTACGTTGAGACCCTGGACGACATCTTTGAGCAGTGGGCTCAC
TCTGAAGACCTGCAGTCCCTCCTGCTTAGGGTCGCTAATGCTGTT
TCGGTGAAAGGGATTTATAAACAGATGCCTGGTTGCTTTAATTTC
CTCCGGAAAAAACTTTTCTTTAAAACATCAGCTAGCAGAGCCGAG
GGCAGGGGAAGTCTTCTAACATGCGGGGACGTGGAGGAAAATCCC
GGGCCCATGGAGTTTGGGCTGAGCTGGCTTTTTCTTGTGGCTATT
TTAAAAGGTGTCCAGTGCTCTAGAGATATTTTGCTGACCCAAACT
CCACTCTCCCTGCCTGTCAGTCTTGGAGATCAAGCCTCCATCTCT
TGCAGATCTAGTCAGAGTCTTGTACACCGTAATGGAAACACCTAT
TTACATTGGTACCTGCAGAAGCCAGGCCAGTCTCCAAAGCTCCTG
ATTCACAAAGTTTCCAACCGATTTTCTGGGGTCCCAGACAGGTTC
AGTGGCAGTGGATCAGGGACAGATTTCACACTCAAGATCAGCAGA
GTGGAGGCTGAGGATCTGGGAGTTTATTTCTGTTCTCAAAGTACA
CATGTTCCTCCGCTCACGTTCGGTGCTGGGACCAAGCTGGAGCTG
AAACGGGCTGATGCTGCACCAACTGTATCCATCTTCCCAGGCTCG
GGCGGTGGTGGGTCGGGTGGCGAGGTGAAGCTTCAGCAGTCTGGA
CCTAGCCTGGTGGAGCCTGGCGCTTCAGTGATGATATCCTGCAAG
GCTTCTGGTTCCTCATTCACTGGCTACAACATGAACTGGGTGAGG
CAGAACATTGGAAAGAGCCTTGAATGGATTGGAGCTATTGATCCT
TACTATGGTGGAACTAGCTACAACCAGAAGTTCAAGGGCAGGGCC
ACATTGACTGTAGACAAATCGTCCAGCACAGCCTACATGCACCTC
AAGAGCCTGACATCTGAGGACTCTGCAGTCTATTACTGTGTAAGC
GGAATGGAGTACTGGGGTCAAGGAACCTCAGTCACCGTCTCCTCA
GCCAAAACGACACCCCCATCAGTCTATGGAAGGGTCACCGTCTCT
TCAGCGGAGCCCAAATCTTGTGACAAAACTCACACATGCCCACCG
TGCCCGGATCCCAAATTTTGGGTGCTGGTGGTGGTTGGTGGAGTC
CTGGCTTGCTATAGCTTGCTAGTAACAGTGGCCTTTATTATTTTC
TGGGTGAGGAGTAAGAGGAGCAGGCTCCTGCACAGTGACTACATG
AACATGACTCCCCGCCGCCCCGGGCCCACCCGCAAGCATTACCAG
CCCTATGCCCCACCACGCGACTTCGCAGCCTATCGCTCCAAACGG
GGCAGAAAGAAACTCCTGTATATATTCAAACAACCATTTATGAGA
CCAGTACAAACTACTCAAGAGGAAGATGGCTGTAGCTGCCGATTT
CCAGAAGAAGAAGAAGGAGGATGTGAACTGAGAGTGAAGTTCAGC
AGGAGCGCAGACGCCCCCGCGTACCAGCAGGGCCAGAACCAGCTC
TATAACGAGCTCAATCTAGGACGAAGAGAGGAGTACGATGTTTTG
GACAAGAGACGTGGCCGGGACCCTGAGATGGGGGGAAAGCCGAGA
AGGAAGAACCCTCAGGAAGGCCTGTACAATGAACTGCAGAAAGAT
AAGATGGCGGAGGCCTACAGTGAGATTGGGATGAAAGGCGAGCGC
CGGAGGGGCAAGGGGCACGATGGCCTTTACCAGGGTCTCAGTACA
GCCACCAAGGACACCTACGACGCCCTTCACATGCAGGCCCTGCCC
CCTCGCTAA.

Namely, the nucleotide sequence SEQ ID NO: 50 comprises the following sequences:

iCasp9 sequence—the inducible caspase-9 expression cassette, encoded by the nucleotide sequence:

(SEQ ID NO: 33)
ATGCTCGAGGGAGTGCAGGTGGAGACTATCTCCCCAGGAGACGGG
CGCACCTTCCCCAAGCGCGGCCAGACCTGCGTGGTGCACTACACC
GGGATGCTTGAAGATGGAAAGAAAGTTGATTCCTCCCGGGACAGA
AACAAGCCCTTTAAGTTTATGCTAGGCAAGCAGGAGGTGATCCGA
GGCTGGGAAGAAGGGGTTGCCCAGATGAGTGTGGGTCAGAGAGCC
AAACTGACTATATCTCCAGATTATGCCTATGGTGCCACTGGGCAC
CCAGGCATCATCCCACCACATGCCACTCTCGTCTTCGATGTGGAG
CTTCTAAAACTGGAATCTGGCGGTGGATCCGGAGTCGACGGATTT
GGTGATGTCGGTGCTCTTGAGAGTTTGAGGGGAAATGCAGATTTG
GCTTACATCCTGAGCATGGAGCCCTGTGGCCACTGCCTCATTATC
AACAATGTGAACTTCTGCCGTGAGTCCGGGCTCCGCACCCGCACT
GGCTCCAACATCGACTGTGAGAAGTTGCGGCGTCGCTTCTCCTCG
CTGCATTTCATGGTGGAGGTGAAGGGCGACCTGACTGCCAAGAAA
ATGGTGCTGGCTTTGCTGGAGCTGGCGCgGCAGGACCACGGTGCT
CTGGACTGCTGCGTGGTGGTCATTCTCTCTCACGGCTGTCAGGCC
AGCCACCTGCAGTTCCCAGGGGCTGTCTACGGCACAGATGGATGC
CCTGTGTCGGTCGAGAAGATTGTGAACATCTTCAATGGGACCAGC
TGCCCCAGCCTGGGAGGGAAGCCCAAGCTCTTTTTCATCCAGGCC
TGTGGTGGGGAGCAGAAAGACCATGGGTTTGAGGTGGCCTCCACT
TCCCCTGAAGACGAGTCCCCTGGCAGTAACCCCGAGCCAGATGCC
ACCCCGTTCCAGGAAGGTTTGAGGACCTTCGACCAGCTGGACGCC
ATATCTAGTTTGCCCACACCCAGTGACATCTTTGTGTCCTACTCT
ACTTTCCCAGGTTTTGTTTCCTGGAGGGACCCCAAGAGTGGCTCC
TGGTACGTTGAGACCCTGGACGACATCTTTGAGCAGTGGGCTCAC
TCTGAAGACCTGCAGTCCCTCCTGCTTAGGGTCGCTAATGCTGTT
TCGGTGAAAGGGATTTATAAACAGATGCCTGGTTGCTTTAATTTC
CTCCGGAAAAAACTTTTCTTTAAAACATCAGCTAGC,
which is linked by a linker AGAGCC to
a T2A Peptide (2A) sequence:
(SEQ ID NO: 34)
GAGGGCAGGGGAAGTCTTCTAACATGCGGGGACGTGGAGGAAAAT
CCCGGGCCC,
a Signal peptide:
(SEQ ID NO: 35)
ATGGAGTTTGGGCTGAGCTGGCTTTTTCTTGTGGCTATTTTAAAA
GGTGTCCAGTGCTCTAGA,
Anti GD2(14G2a) VL sequence:
(SEQ ID NO: 36)
GATATTTTGCTGACCCAAACTCCACTCTCCCTGCCTGTCAGTCTT
GGAGATCAAGCCTCCATCTCTTGCAGATCTAGTCAGAGTCTTGTA
CACCGTAATGGAAACACCTATTTACATTGGTACCTGCAGAAGCCA
GGCCAGTCTCCAAAGCTCCTGATTCACAAAGTTTCCAACCGATTT
TCTGGGGTCCCAGACAGGTTCAGTGGCAGTGGATCAGGGACAGAT
TTCACACTCAAGATCAGCAGAGTGGAGGCTGAGGATCTGGGAGTT
TATTTCTGTTCTCAAAGTACACATGTTCCTCCGCTCACGTTCGGT
GCTGGGACCAAGCTGGAGCTGAAACGGGCTGATGCTGCACCAACT
GTATCCATCTTCCCA,
Anti GD2(14G2a) VH sequence:
(SEQ ID NO: 37)
GAGGTGAAGCTTCAGCAGTCTGGACCTAGCCTGGTGGAGCCTGGC
GCTTCAGTGATGATATCCTGCAAGGCTTCTGGTTCCTCATTCACT
GGCTACAACATGAACTGGGTGAGGCAGAACATTGGAAAGAGCCTT
GAATGGATTGGAGCTATTGATCCTTACTATGGTGGAACTAGCTAC
AACCAGAAGTTCAAGGGCAGGGCCACATTGACTGTAGACAAATCG
TCCAGCACAGCCTACATGCACCTCAAGAGCCTGACATCTGAGGAC
TCTGCAGTCTATTACTGTGTAAGCGGAATGGAGTACTGGGGTCAA
GGAACCTCAGTCACCGTCTCCTCA,
The G7S2 linker (which links anti GD2 (14G2a)
VL sequence and anti GD2 (14G2a) VH sequence)
sequence:
(SEQ ID NO: 44)
GGCTCGGGCGGTGGTGGGTCGGGTGGC;
IgG-based hinge sequence:
(SEQ ID NO: 45)
GCCAAAACGACACCCCCATCAGTCTATGGAAGGGTCACCGTCTCT
TCAGCGGAGCCCAAATCTTGTGACAAAACTCACACATGCCCACCG
TGCCCG;
which is linked by the Linker (connection
sequence) GATCCCAAA to a transmembrane domain
CD28TM sequence:
(SEQ ID NO: 46)
TTTTGGGTGCTGGTGGTGGTTGGTGGAGTCCTGGCTTGCTATAGC
TTGCTAGTAACAGTGGCCTTTATTATTTTCTGGGTG;
CD28 cytoplasmic sequence:
(SEQ ID NO: 47)
AGGAGTAAGAGGAGCAGGCTCCTGCACAGTGACTACATGAACATG
ACTCCCCGCCGCCCCGGGCCCACCCGCAAGCATTACCAGCCCTAT
GCCCCACCACGCGACTTCGCAGCCTATCGCTCC
CD137 (4-1BB) sequence:
(SEQ ID NO: 48)
AAACGGGGCAGAAAGAAACTCCTGTATATATTCAAACAACCATTT
ATGAGACCAGTACAAACTACTCAAGAGGAAGATGGCTGTAGCTGC
CGATTTCCAGAAGAAGAAGAAGGAGGATGTGAACTG
CD3-Zeta chain sequence:
(SEQ ID NO: 49)
AGAGTGAAGTTCAGCAGGAGCGCAGACGCCCCCGCGTACCAGCAG
GGCCAGAACCAGCTCTATAACGAGCTCAATCTAGGACGAAGAGAG
GAGTACGATGTTTTGGACAAGAGACGTGGCCGGGACCCTGAGATG
GGGGGAAAGCCGAGAAGGAAGAACCCTCAGGAAGGCCTGTACAAT
GAACTGCAGAAAGATAAGATGGCGGAGGCCTACAGTGAGATTGGG
ATGAAAGGCGAGCGCCGGAGGGGCAAGGGGCACGATGGCCTTTAC
CAGGGTCTCAGTACAGCCACCAAGGACACCTACGACGCCCTTCAC
ATGCAGGCCCTGCCCCCTCGCTAA.

According to the present invention, said vector can be chosen from a DNA vector, a RNA vector, a plasmid, a lentivirus vector, adenoviral vector, retrovirus vector, such as γ-retroviral vector, or non-viral vector.

In addition, the present invention also concerns a cell, such as T cell, such as alfa/beta and gamma/delta T cell, NK cells, NK-T cells as well as macrophages or monocyte cells, comprising the anti-GD2 chimeric antigen receptor according claims 35-44 and/or the vector according to claims 45-50. According to the invention, said cell can further comprise a suicide gene inducible amino acid sequence such as a chimeric Caspase-9 polypeptide or a herpes simplex virus thymidine kinase (HSV-TK) or ACD19 sequence as a safety switch. In particular, the chimeric Caspase-9 polypeptide can comprise or consist of:

iCasp9 cassette:
(SEQ ID NO: 1)
MLEGVQVETISPGDGRTFPKRGQTCVVHYTGMLEDGKKVDSSRDR
NKPFKFMLGKQEVIRGWEEGVAQMSVGQRAKLTISPDYAYGATGH
PGIIPPHATLVFDVELLKLESGGGSGVDGFGDVGALESLRGNADL
AYILSMEPCGHCLIINNVNFCRESGLRTRTGSNIDCEKLRRRFSS
LHFMVEVKGDLTAKKMVLALLELARQDHGALDCCVVVILSHGCQA
SHLQFPGAVYGTDGCPVSVEKIVNIFNGTSCPSLGGKPKLFFIQA
CGGEQKDHGFEVASTSPEDESPGSNPEPDATPFQEGLRTFDQLDA
ISSLPTPSDIFVSYSTFPGFVSWRDPKSGSWYVETLDDIFEQWAH
SEDLQSLLLRVANAVSVKGIYKQMPGCFNFLRKKLFFKTSAS,
which is linked by a linker, such as RA, to:
a T2A peptide (2A):
(SEQ ID NO: 2)
EGRGSLLTCGDVEENPGP.

According to the present invention, said cell can be obtained in culture conditions wherein both or each of IL-7 and/or IL-15 are present, for example in the culture conditions of the activation step, transduction step and/or expansion step of the process for the preparation of said cell.

The present invention concerns also a pharmaceutical composition comprising the nucleotide sequence or vector according to claims 45-50, or the cell according to claims 51-53 together with one or more pharmaceutically acceptable excipients and/or adjuvants.

The present invention also concerns a kit of parts comprising or consisting of:
A) an anti-GD2 chimeric antigen receptor as defined in any one of claims 35-44, or a nucleotide sequence or a vector according to any one of claims 45-50, or a cell according to any one of claims 51-53 or a pharmaceutical composition according to claim 54; and
B) an enhancer of Zeste Homolog 1 or 2 (EZH2) inhibitor or a pharmaceutical composition comprising an Enhancer of Zeste Homolog 1 or 2 (EZH2) inhibitor and one or more excipients and/or adjuvants,
wherein A) and B) are to administered separately or sequentially.

According to the invention, said Enhancer of Zeste Homolog 2 inhibitor can be chosen from the group consisting of Tazemetostat, CPI-1205, which is an orally bioavailable, indole-based, small-molecule inhibitor of EZH268 69; PF-0682149770, GSK126 (GSK2816126) 71, preferably Tazemetostat.

Moreover, the present invention concerns an anti-GD2 chimeric antigen receptor according to claims 35-44, a nucleotide sequence or vector according to any one of claims 45-50, a cell according to any one of claims 51-53, a pharmaceutical composition according to claim 54 or kit according to any one of claims 55-56, for medical use.

In particular, said anti-GD2 chimeric antigen receptor, nucleotide sequence, vector, cell, pharmaceutical composition or kit can be for use in the treatment of neuroblastoma, diffuse midline glioma (DMG) H3K27M-mutant, including diffuse intrinsic pontine glioma (DIPG) and osteosarcoma.

According to an embodiment of the invention, said use is also for the treatment of high grade glioma. According to an embodiment of the invention, said use is also for rhabdomyosarcoma or Ewing sarcoma. According to a further embodiment of the present invention, said use is also for sarcoma, for example osteosarcoma, rhabdomyosarcoma or Ewing sarcoma. According to an embodiment of the present invention, said use is also for the treatment of medulloblastoma. According to an embodiment of the invention, said anti-GD2 chimeric antigen receptor is for use in the treatment of glioma.

In other words, said anti-GD2 chimeric antigen receptor, nucleotide sequence, vector, cell or pharmaceutical composition, can be for use in the treatment of all the solid tumor, in particular a solid tumor having an expression of GD2 higher than the expression of GD2 in healthy peripheral blood mononuclear cells, wherein the expression of GD2 is measured as the percentage of GD2+ cells with respect to the total number of cells of the tumor or of the healthy peripheral blood mononuclear cells.

The present invention now will be described by an illustrative, but not limitative way, according to preferred embodiments thereof, with particular reference to the examples and the enclosed drawings, wherein:

FIG. 1(A-E). iC9.2A.GD2.CAR-28.4-1BB.ζ T(NK)-cells with CD28.4-1BB co-stimulation exhibit high and stable transduction level and in vitro proliferation upon initial antigen stimulation. (A) The expression cassette of SFG.iC9.2A.CAR-GD2 (14g2a) CD8tm.CD28.41BBζ (iC9.2A.GD2.CAR-28.4-1BB.ζ) retroviral vector shown in cartoon 1A. The scFv of GD2 was cloned in frame with CD8aTM, CD28 cytoplasmic moiety, and a second costimulatory domain represented by 4-1BB and the signalling domain CD3-zeta chain (2). (B) Flow-cytometry analyses shows the level of transduction of T cells by expression in an exemplificative donor, growth in IL7/IL15, of un-transduced (NT) T-cells, as negative control (1B, left panels), or GD2.CAR-28.4-1BBζ (GD2.CAR) T-cells, transduced with iC9.2A.GD2.CAR-28.4-1BB.ζ retroviral vector (1B, right panels). (C) The panel 1C shows the average of the percentage of positive GD2.CAR T-cells, profiled by FACS. Data are expressed as average±standard deviation (SD) from six healthy donors (HDs) at day 15 of in vitro culture. (D) Graph show the fold expansion in IL7/IL15 (D) of NT T-cells and GD2.CAR T-cells, evaluated by trypan-blue count assay. The panel (E) shows the average of the percentage of positive GD2.CAR NK-cells, profiled by FACS at day 5 and day+25 of in vitro culture. Data are expressed as average±standard deviation (SD) from six healthy donors (HDs). * p-value=<0.05, ** p-value=<0.01, *** p-value=<0.001 and **** p-value=<0.0001.

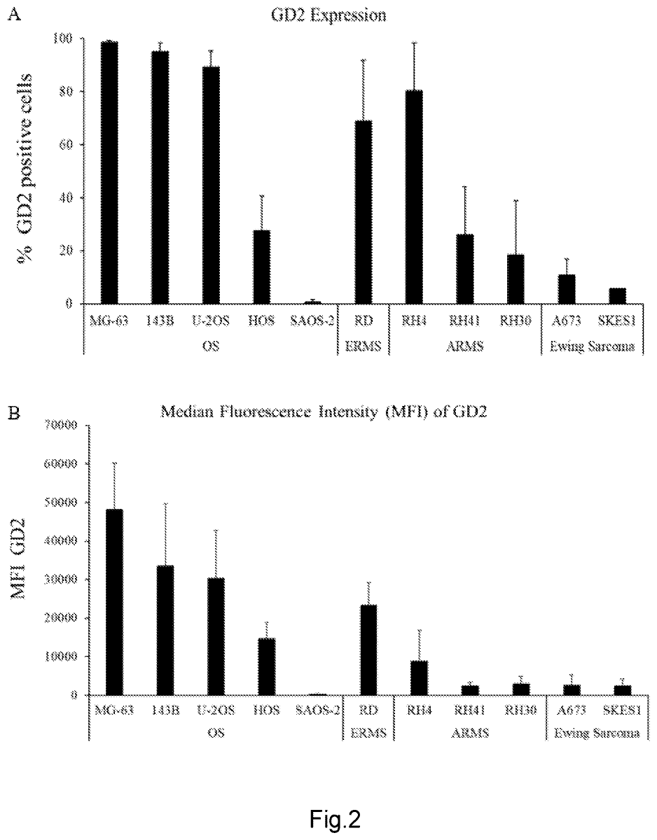

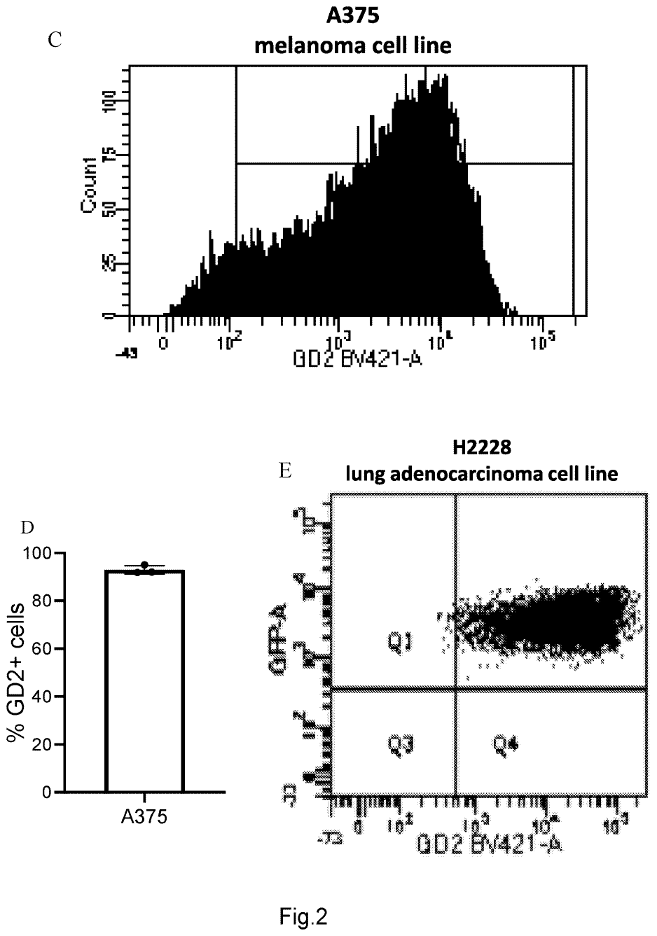

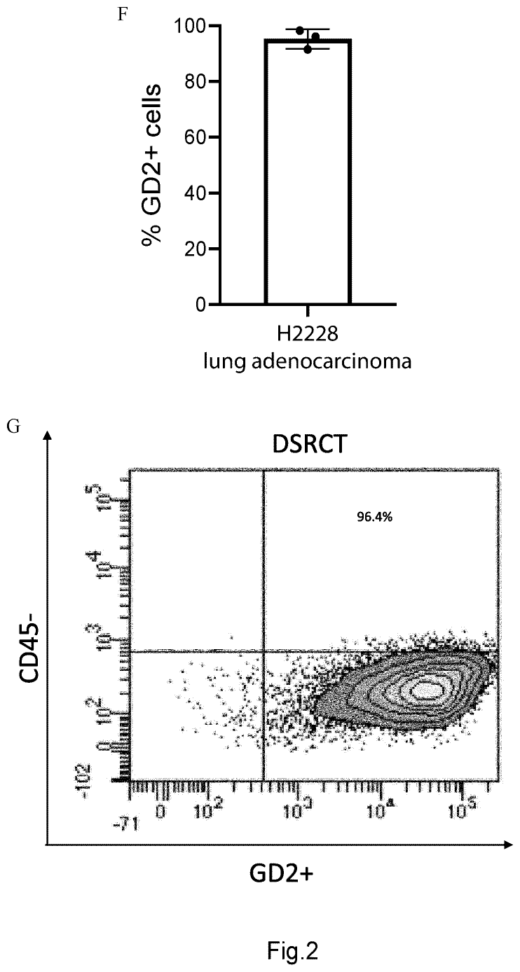

FIG. 2(A-B). GD2 Expression in human sarcoma cell lines. (A) Expression of GD2 (as percentage) in human osteosarcoma (OS) cell lines: 143B, MG-63, U-20S, HOS and SAOS-2; in embryonal rhabdomyosarcoma (ERMS) cell lines: RD; in Alveolar Rhabdomyosarcoma (ARMS) cell lines: RH4, RH41 and RH30 and in Ewing Sarcoma (EwS) cell lines: A673 and SKES1, as assessed by flow cytometry. Data are expressed as percentage±SD from 4 different replicates. (B) Median Fluorescence Intensity (MFI) of GD2 expression in human sarcoma cell lines. Data are expressed as mean±SD from 4 different replicates. (C-D) GD2 Expression in melanoma cell line. (C) Representative FACS analysis of GD2 expression on melanoma A375 cell line (CRL-1619/ATCC). (D) flow-cytometry analyses were assessed in triplicate. Data are shown as average±SD. (E-F). GD2 Expression in lung adenocarcinoma cell line. (C) Representative FACS analysis of GD2 expression on lung adenocarcinoma cell line H2228 cell line (CRL-5935/ATCC). (D) flow-cytometry analyses were assessed in triplicate. Data are shown as average±SD. (G). GD2 Expression in primary desmoplastic small round cell tumors (DSRCT) tumors. GD2 expression on pediatric DSRCT tissues, evaluated by facs analysis (as CD45^{neg}GD2+).

FIG. 3(A-J). Long-term in vitro co-culture assays to evaluate functional activities of GD2.CAR-28.4-1BBζ (GD2.CAR) T-cells. Residual tumor was expressed as percentage of remaining tumor cells, after 5 days in co-culture experiments with NT or GD2.CAR-28.4-1BBζ T-cells at the E:T ratio 1:1; with NT (black bar) GD2.CAR-28.4-1BBζ T-cells (white bar). The tumor cells: MG63 tumor cell line (A), 143B cell line (B), U-20S cell line (C), HOS cell line (D) RD cell line (E) RH4cell line (F), RH41 cell line (G), A673 cell line (H), A375 cell line (I) and H2228 cell line (J) were used as targets. Data from 6 Healthy Donors (HDs) are expressed as average±SD in A-J; *p-value=<0.05; **p-value=<0.01; ***p-value=<0.001 and ****p-value=<0.0001.

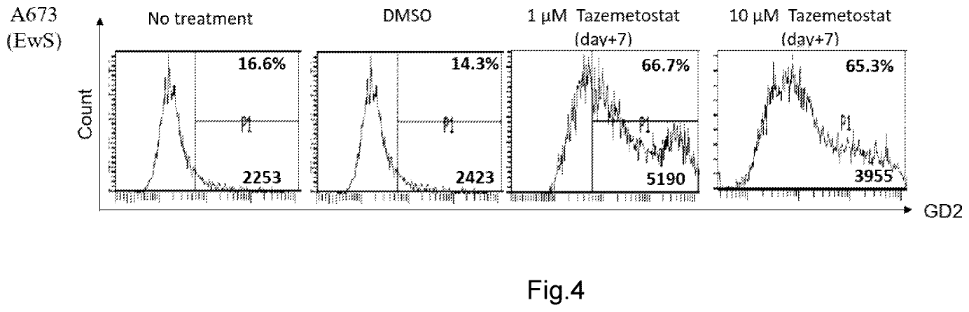

FIG. 4 Inhibition of EZH2 upregulates surface GD2 expression in EwS cells. Flow-cytometry analyses shows GD2 expression (percentage and MFI) in human EwS cells treated with 1-10 UM of tazemetostat for 7 days. DMSO was included as negative control.

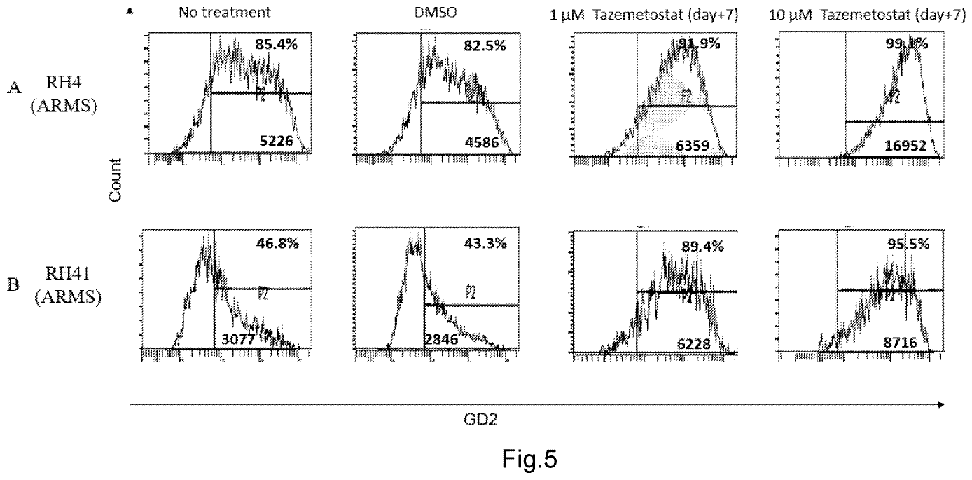

FIG. 5(A-B) Inhibition of EZH2 upregulates surface GD2 expression in ARMS cells. Flow-cytometry analyses shows GD2 expression (percentage and MFI) in RH4 (A) and RH41 (B) cell lines treated with 1-10 UM of tazemetostat for 7 days. DMSO was included as negative control.

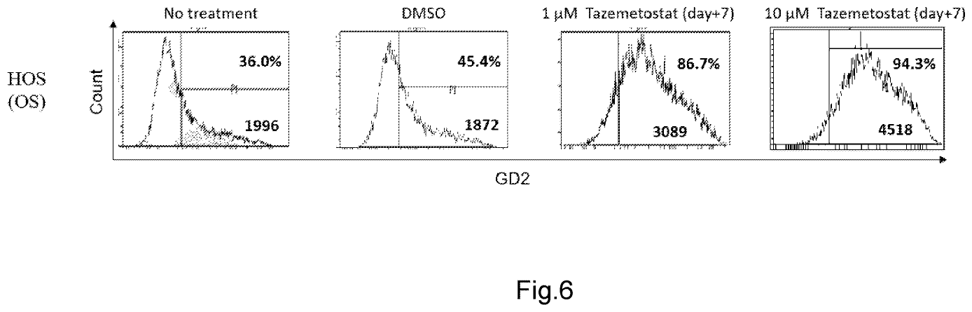

FIG. 6 Inhibition of EZH2 upregulates surface GD2 expression in Osteosarcoma (OS) cells. Flow-cytometry analyses shows GD2 expression (percentage and MFI) in HOS cell line treated with 1-10 UM of Tazemetostat for 7 days. DMSO was included as negative control.

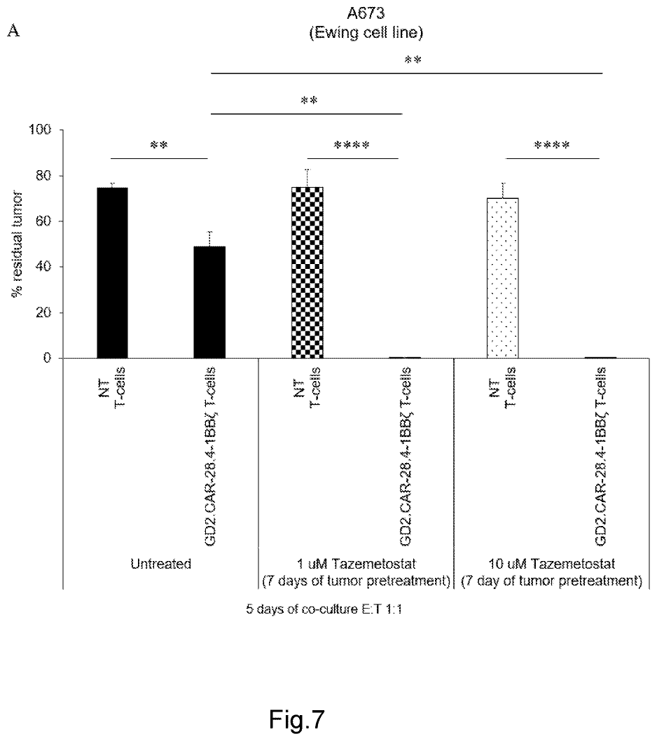

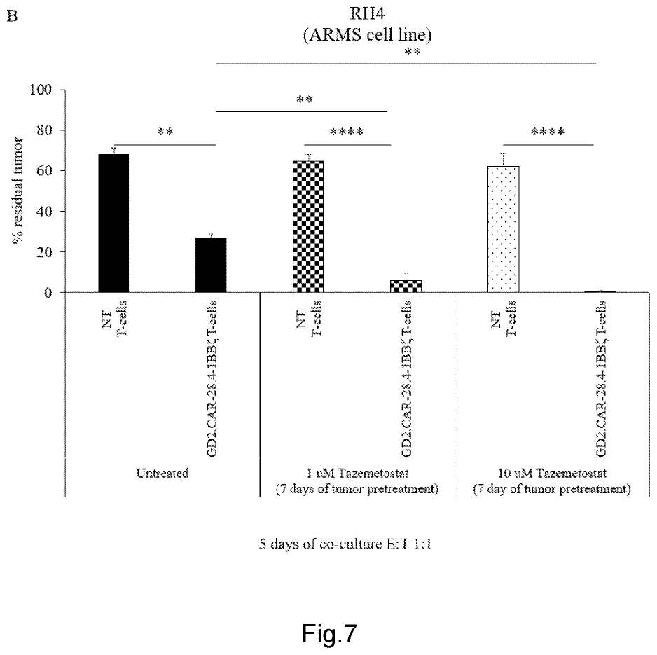

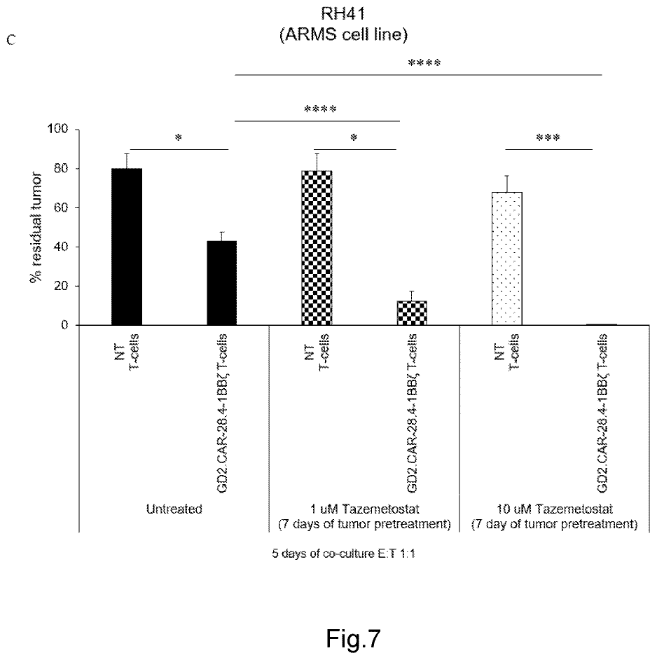

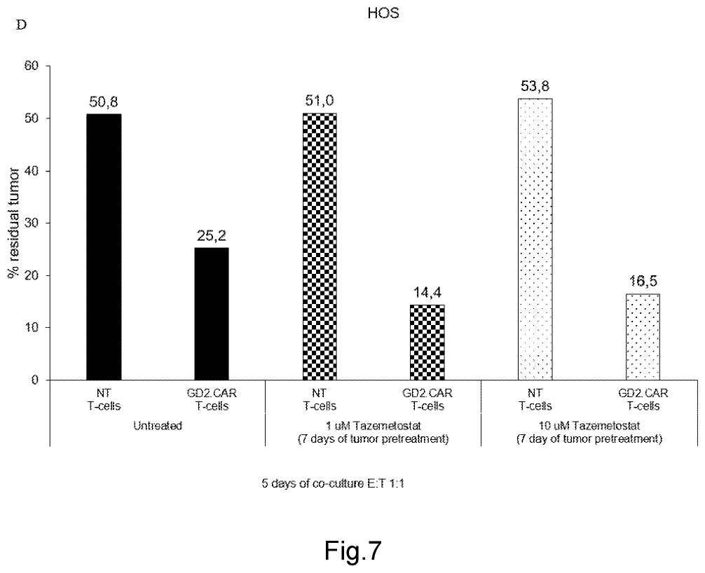

FIG. 7(A-D). EZH2 Inhibitor pretreatment sensitizes both EwS, RMS and OS cell lines to In Vitro Cytolysis by GD2.CAR-28.4-1BBζ (GD2.CAR) T-cells. Residual tumor was expressed as percentage of remaining tumor cells, after 5 days of co-culture experiments between NT or GD2.CAR-28.4-1BBζ T-cells with sarcoma cell lines pre-treated for seven days with 1-10 UM Tazemetostat, at the E:T ratio of 1:1. Black bars indicate sarcoma cells non treated with the drug tazemetostat; Checkerboard bars indicates sarcoma cell lines pre-treated with 1 μM of Tazemetostat, and black dot bars indicates sarcoma cell lines pre-treated with 10UM of Tazemetostat. The sarcoma cells A-673 (Ewing sarcoma) (A), RH4 (ARMS) (B), RH41 (ARMS) (C) and HOS (OS) (D) cell lines were used as targets. Data from 4 HDs are expressed as average±SD in A-H; *p-value=<0.05; **p-value=<0.01; ***p-value=<0.001 and ****p-value=<0.0001

FIG. 8(A-I). In vivo bioluminescence imaging of NSG mice bearing i.v. GD2+RD-FF-Luc.GFP cells treated with NT, or GD2.CAR-28.4-1BBζ T-cells generated and expanded in the presence of IL7/IL15. (A) Schematic model of in vivo experiments. Mice received i.v. 0.5×106 RD-FF-Luc.GFP cells and after 3 days, when the bioluminescence became stable, they were divided in two cohorts and treated with NT or GD2.CAR-28.4-1BBζ T-cells. The tumour growth was evaluated weekly by IVIS for 76 days. (B) Bioluminescence imaging of tumor growth measured weekly from day 3 until day 76; (C) bioluminescence of each single mouse treated with NT (black line; 10 mice) and IIICAR.GD2 T-cells (black dotted line; 10 mice). (D) Graph show average of tumor bioluminescence in mice treated with NT (black line) or IIICAR.GD2 T-cells (black dotted line). (E) Kaplan-Meier estimate overall survival (OS) of tumor-bearing mice treated with either NT (black line; 10 mice) or GD2.CAR-28.4-1BBζ T-cells (black dotted line; 10 mice). * p<0.05; log-rank (Mantel-Cox). (F-I) Average of human circulating T cells evaluated as % of CD45+CD3+ cells (F); as CAR T cells (G), as CD4+ (CD4+CAR+) T cells (H) and CD8+ (CD8+CAR+) T cells (I). *p-value=<0.05; **p-value=<0.01; ***p-value=<0.001; ****p-value=<0.0001, t test.

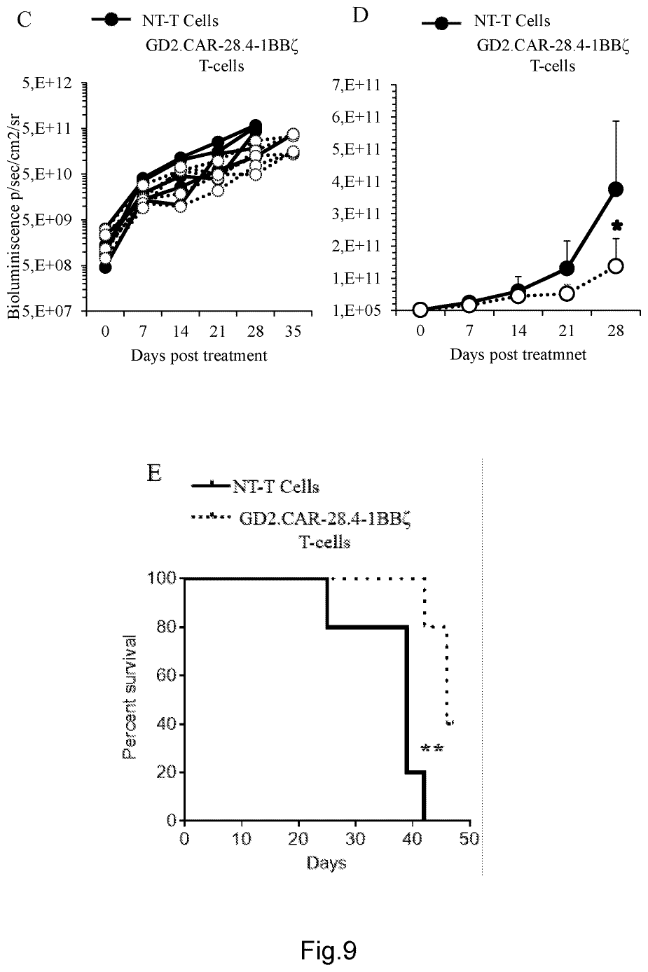

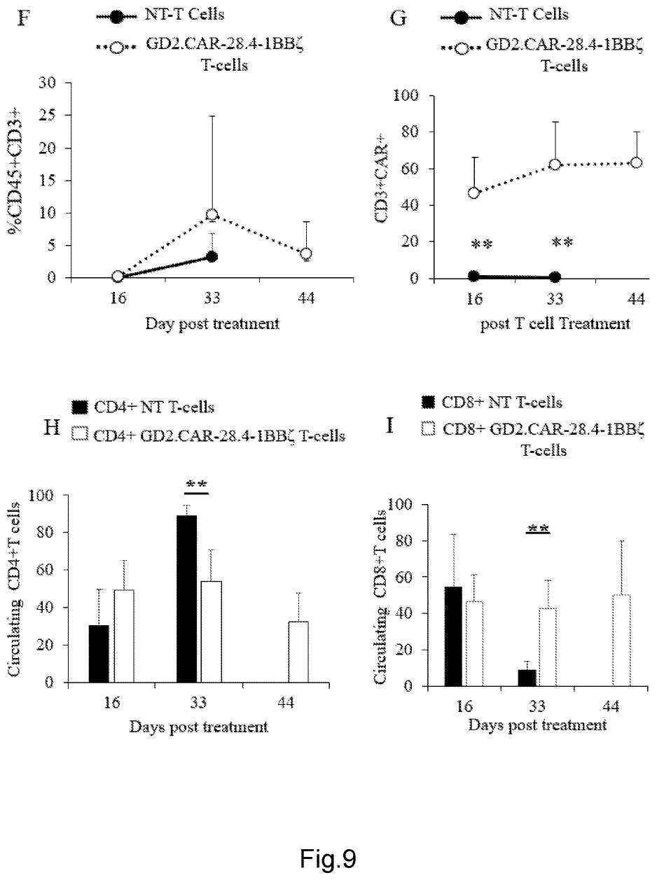

FIG. 9(A-I). In vivo bioluminescence imaging of NSG mice bearing orthotopic GD2+143B-FF-Luc.GFP cells treated with NT, or GD2.CAR-28.4-1BBζ T-cells generated and expanded in the presence of IL7/IL15. (A) Schematic model of in vivo experiments. Mice were inoculated in the tibia with 0.5×106 GD2+143B-FF-Luc.GFP cells and after 3 days, when the bioluminescence became stable, they were divided in two cohorts and treated with NT or GD2.CAR-28.4-1BBζ T-cells. The tumour growth was evaluated weekly by IVIS for 35 days. (B) Bioluminescence imaging of tumor growth measured weekly from day 3 until day 35; (C) bioluminescence of each single mouse treated with NT (black line; 10 mice) and IIICAR. GD2 T-cells (black dotted line; 10 mice). (D) Graph show average of tumor bioluminescence in mice treated with NT (black line) or GD2.CAR-28.4-1BBζ T-cells (black dotted line). (E) Kaplan-Meier estimate overall survival (OS) of tumor-bearing mice treated with either NT (black line; 10 mice) or GD2.CAR-28.4-1BBζ T-cells (black dotted line; 10 mice). **p<0.01; log-rank (Mantel-Cox). (F-I) Average of human circulating T cells evaluated as % of CD45+CD3+ cells (F); as CAR T cells (G), as CD4+ (CD4+CAR+) T cells (H) and CD8+ (CD8+CAR+) T cells (I). *p-value=<0.05; **p-value=<0.01, t test.

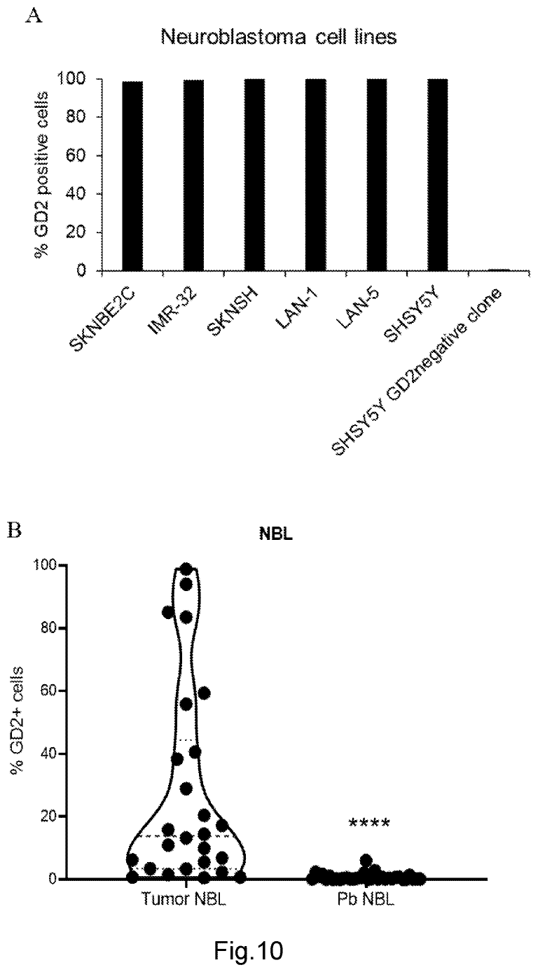

FIG. 10. (A-B). GD2 Expression in human Neuroblastoma cell lines and primary tumors. (A) Percentage of GD2 in human Neuroblastoma cell lines. (B) % GD2 expression in primary Neuroblastoma tumors. Normal periferal blood cells (Pb) were run for each patient as negative control. ****p-value=<0.0001, t test.

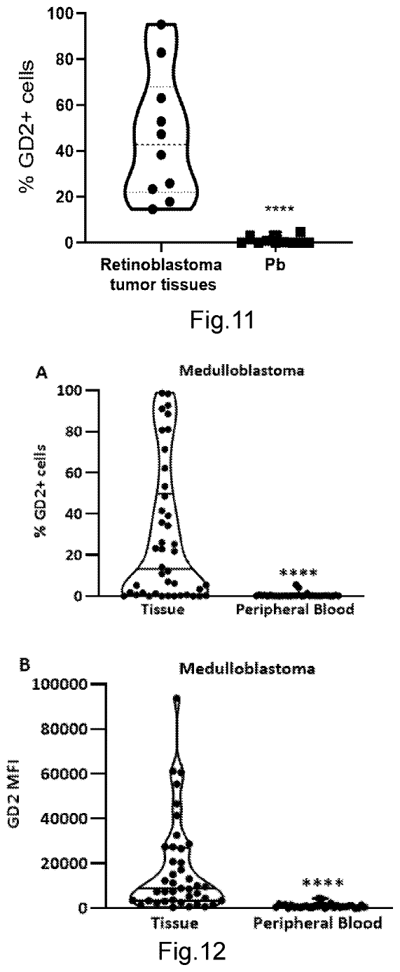

FIG. 11. GD2 Expression in Retinoblastoma tumours derived by primary and patient's tissues. GD2 expression on pediatric retinoblastoma tissues, evaluated by facs analysis (as CD45negGD2+). Normal periferal blood cells (Pb) were run for each patient as negative control. ****p-value=<0.0001, t test.

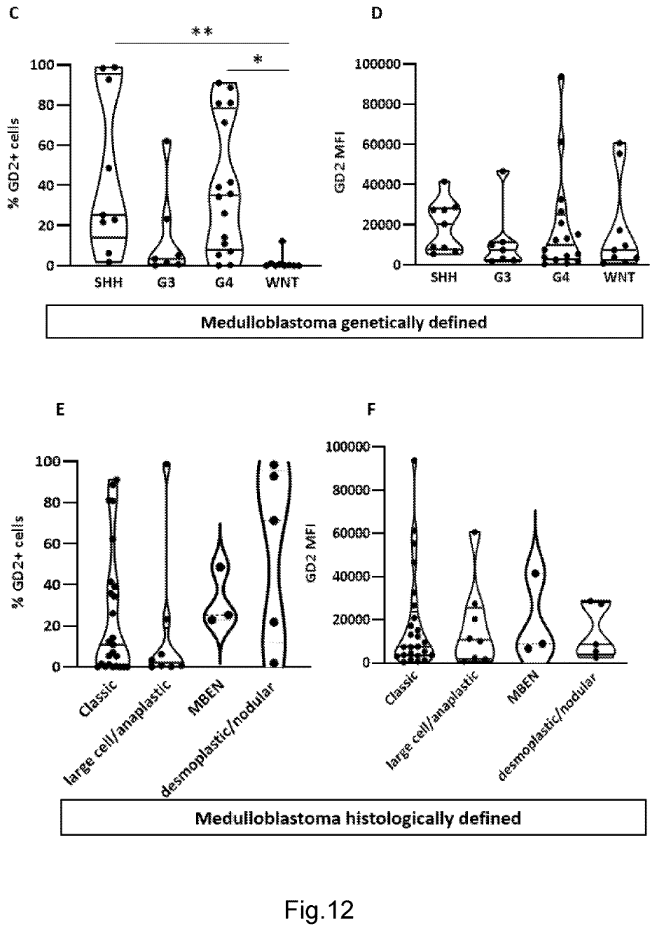

FIG. 12(A-B). GD2 is highly expressed in pediatric MB patients. (A) The percentage of GD2 expression levels on cells of tissues biopsies and PB obtained from pediatric MB patients at diagnosis were assessed by flow-cytometry analyses. (B) MFI for GD2 on cells obtained from tissues biopsies and PB of pediatric MB patients at diagnosis. MFI was adjusted for cells size by dividing MFI by the forward scatter (FSC). (C) The percentage of GD2 positive cells and GD2 MFI (D) was examined in MB samples by considering genetic classification (SHH, G3, G4 and WNT subgroups). (E) The expression levels of GD2 percentage and MFI (F) were examined by considering histologic classification (classic, large cell/anaplastic (LCA), with extensive nodularity (MBEN) and desmoplastic/nodular (DN) subgroups). Data are shown as average±SD. *p<0.05, **p<0.01, ***p≤0.001, ****p≤0.0001.

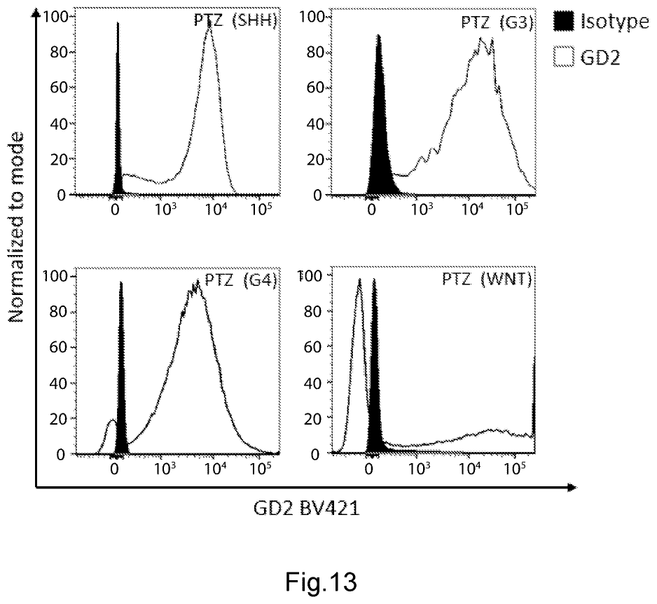

FIG. 13. Representative FACS analysis of GD2 expression in a representative patient of SHH (I plot), G3 (II plot), G4 (III plot) and WNT (IV plot) subgroups. Isotype control is shown in dark grey.

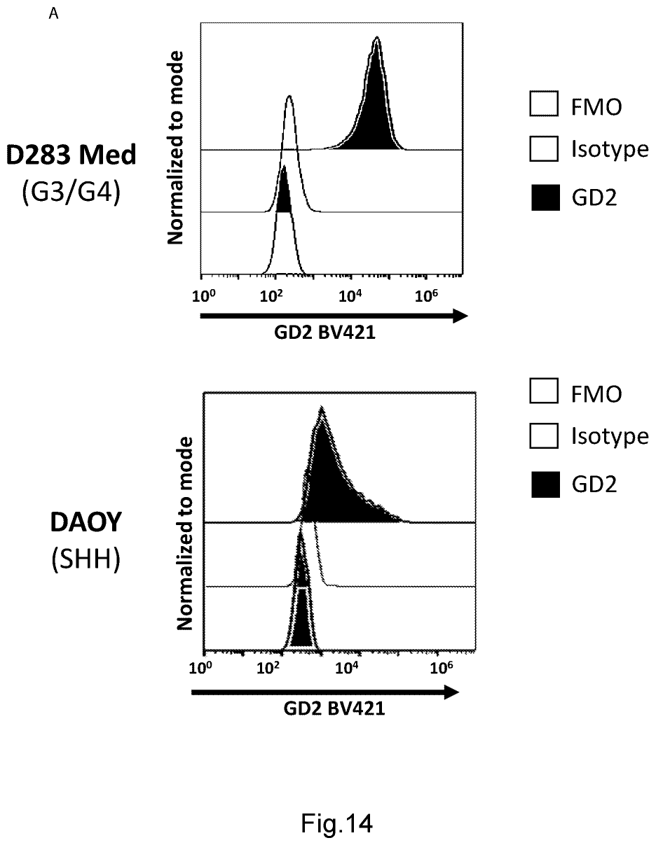

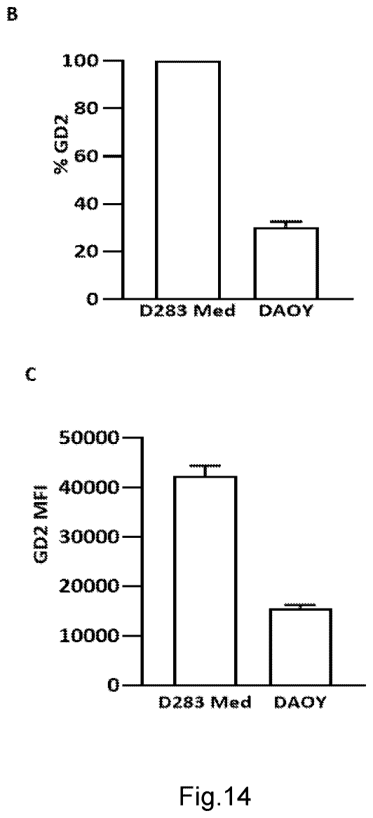

FIG. 14(A-C). GD2 is highly expressed in G3/G4 MB cell lines. (A) Cytofluorimetric analysis of GD2 expression on MB cell lines: D238 Med and DAOY. Fluorescence Minus One (FMO) and isotype controls are shown in white. (B) The percentage of GD2 expression levels on MB cell lines (D238 Med and DAOY) were assessed in triplicate by flow-cytometry analyses. Data are shown as average±SD. (C) MFI for GD2 on MB cell lines (D238 Med and DAOY). MFI was adjusted for cells size by dividing MFI by the forward scatter (FSC). Data are shown as average±SD.

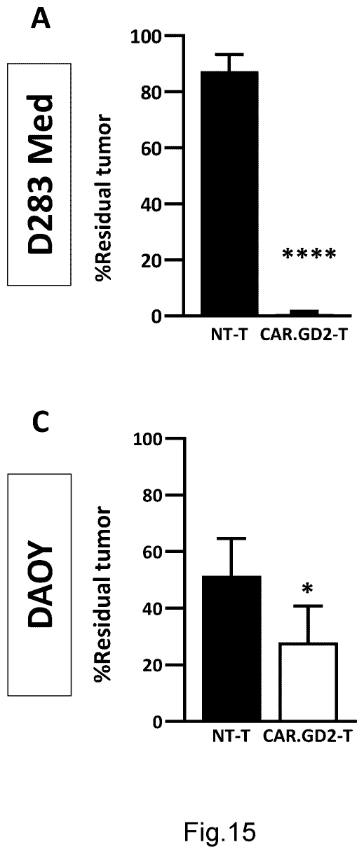

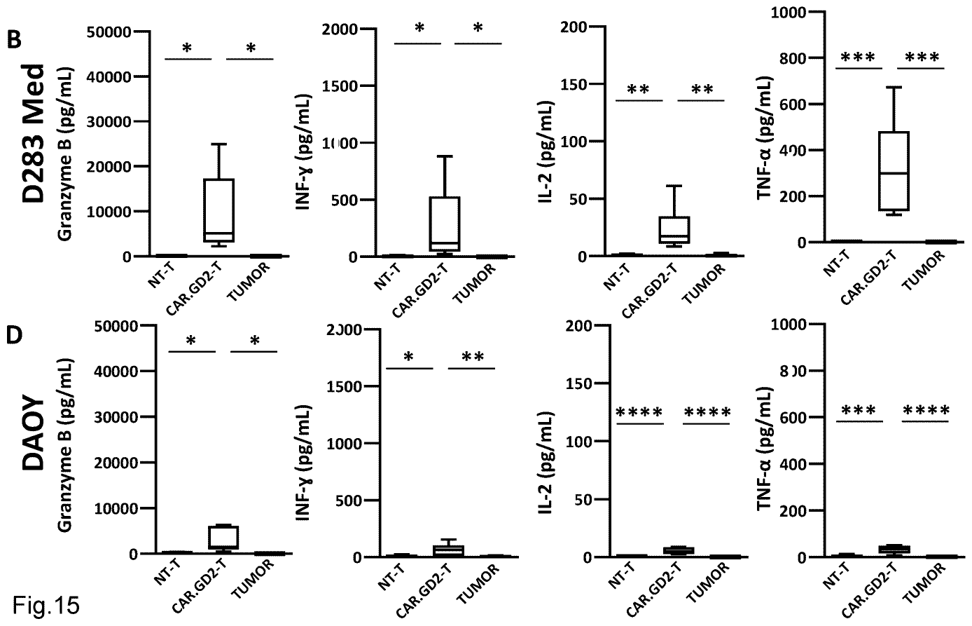

FIG. 15(A-D). CAR.GD2 T-cells target human GD2+MB cell lines in vitro culture. (A, C) Long-term 5-day co-cultures were performed in five independent experiments, in which GD2+MB cell lines D283 Med (A) and DAOY (C) were co-cultured with either NT-T or GD2.CAR-28.4-1BBζ (CAR.GD2) T-cells, derived from HDs, at the E:T ratio of 1:1. The residual tumor was expressed as percentage of GD2+ cells. Data are expressed as average±SD. *p<0.05, **p<0.01, ***p<0.001, ****p<0.0001. (B, D) Granz B, IFN-γ, IL-2 and TNF-α were measured by ELISA assay in 24 h culture supernatant of NT-T or CAR. GD2 T-cells in response to GD2+MB cell lines D283 (B), DAOY (D). Data of cytokine quantification are shown as average±SD. *p<0.05, **p<0.01, ***p<0.001, ****p<0.0001.

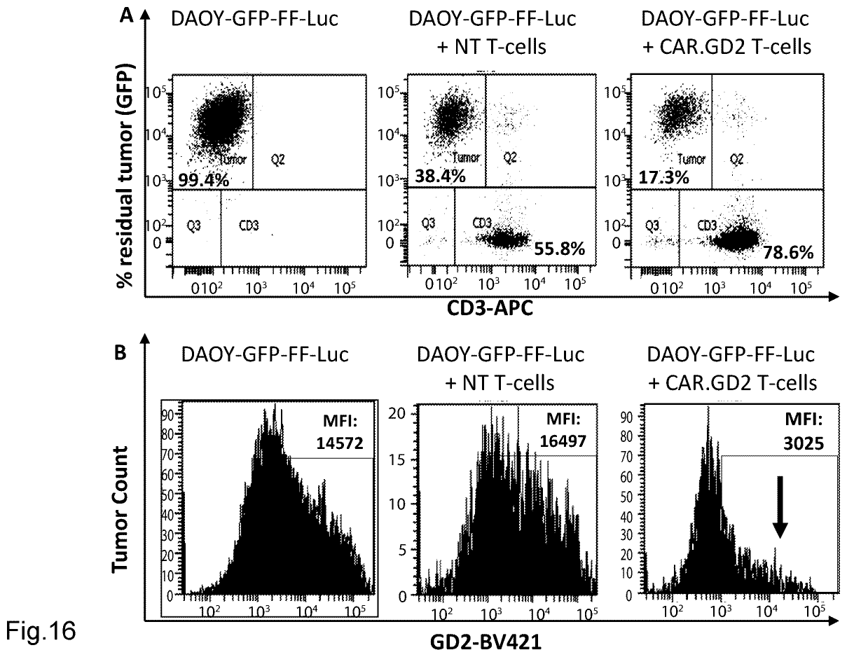

FIG. 16. (A-B) FACS plot of exemplificative experiment. After long-term 5-day co-culture, in which GD2+MB cell lines DAOY was seeded alone or co-cultured with either NT-T or GD2.CAR-28.4-1BBζ (CAR.GD2) T-cells at the E:T ratio of 1:1. The residual tumor was evaluated by FACS analysis and expressed as percentage of residual tumor (A) or GD2 MFI (B).

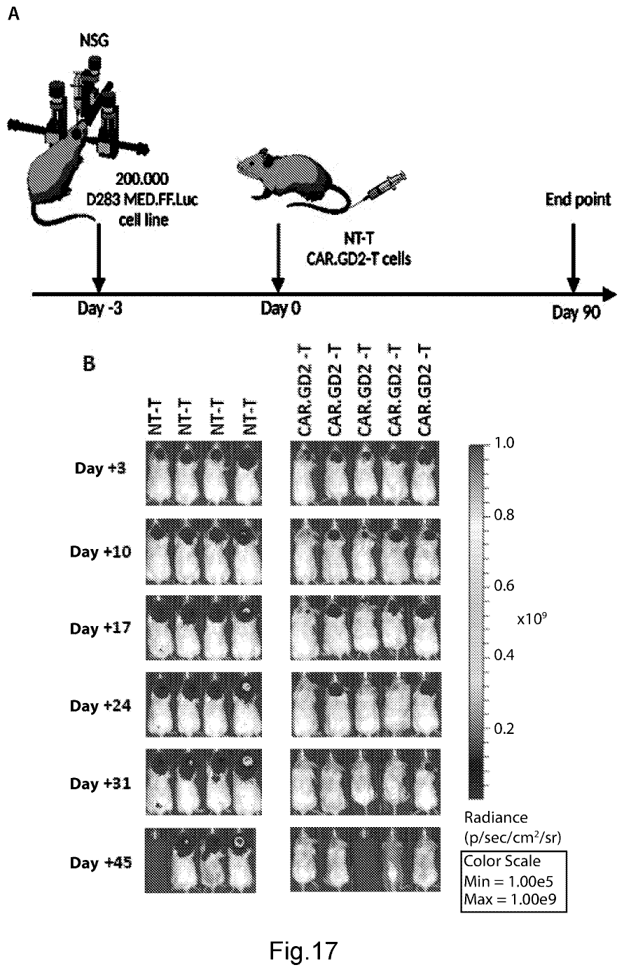

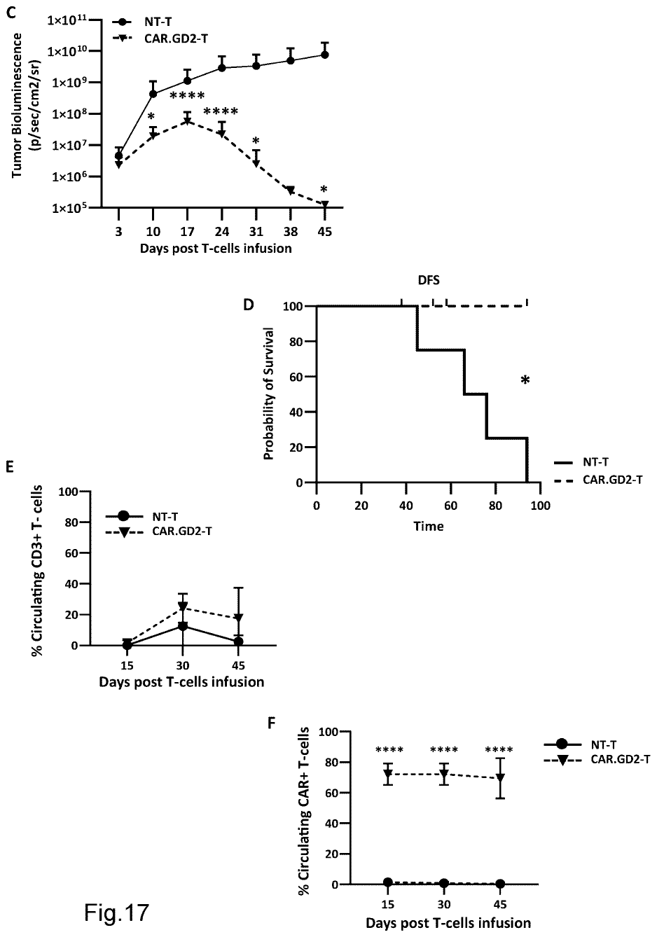

FIG. 17(A-F). Orthotopic mouse model of human D283 Med-GFP-FF-Luc cell line to evaluate anti-tumor activity of GD2.CAR-28.4-1BBζ (CAR.GD2) T-cells. (A) Illustration of experimental setting in which D283 Med-GFP-FF-Luc cell line was stereotaxically implanted into the cerebellum of NSG mice. After 3 days, effector T-cells were administered through iv injection. (B-C) Time course of in vivo bioluminescence imaging of the each treated NSG mice (B) from day 3 (day of effector T-cells infusion). (C) Graph shows average of in vivo bioluminescence of mice treated with NT-T (black line) and CAR. GD2 T-cells (dotted black line). Data are shown as average±SD. *p<0.05, ****p<0.0001. (D) Disease Free Survival (DFS) of tumor bearing NSG mice treated with NT T (black line) or CAR.GD2 T-cells (dotted black line). *p<0.05. (E-F) Average of human circulating T cells evaluated as % of CD45+CD3+ cells (E) and CD3+CAR+ cells (F) in NSG mice treated with NT T-cells (red line) or CAR.GD2-T cells (dotted black line) at day 15, 30 and 45 after effector T-cells infusion. Data are shown as average±SD. ****p<0.0001.

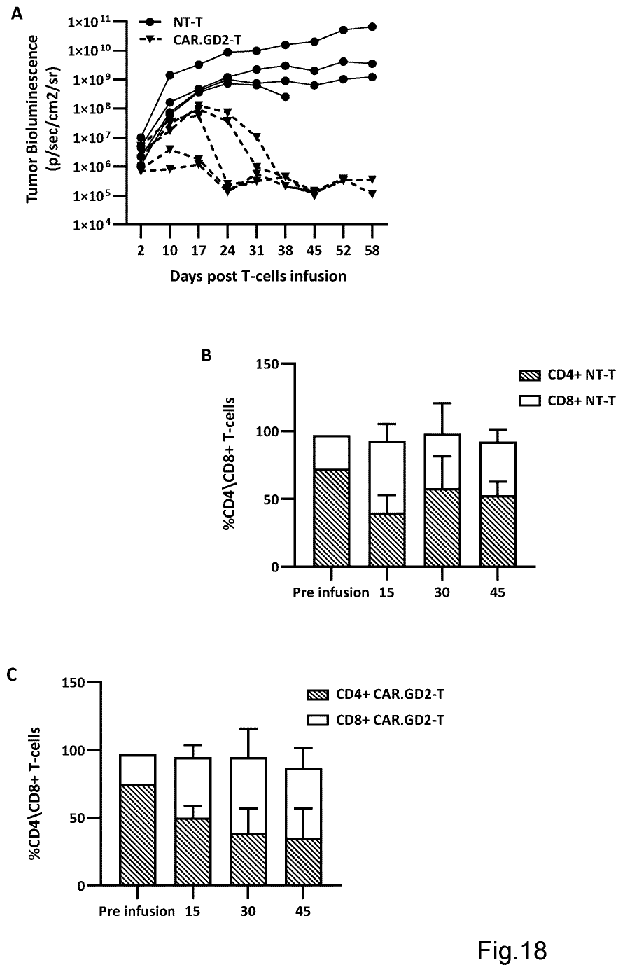

FIG. 18(A-C). Graph shows bioluminescence analysis (A) of each tumor bearing mice treated with NT-T (black lines with circle) or GD2.CAR-28.4-1BBζ (CAR.GD2) T-cells (dotted black lines with triangle). (B) Flow cytometry analysis of CD4⁺ and CD8⁺ subset cells in tumor bearing mice treated with NT-T on day of pre-infusion, on day 15 and day 30. (C) Flow cytometry analysis of CD4⁺ and CD8⁺ subset cells in tumor bearing mice treated with CAR.GD2-T on day of pre-infusion, on day 15 and day 30. Data are shown as average±SD.

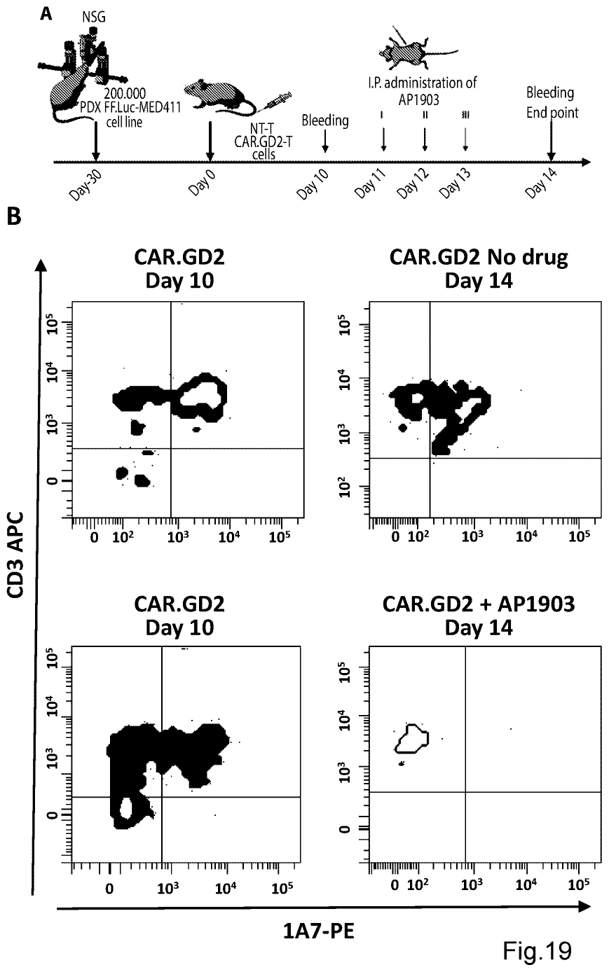

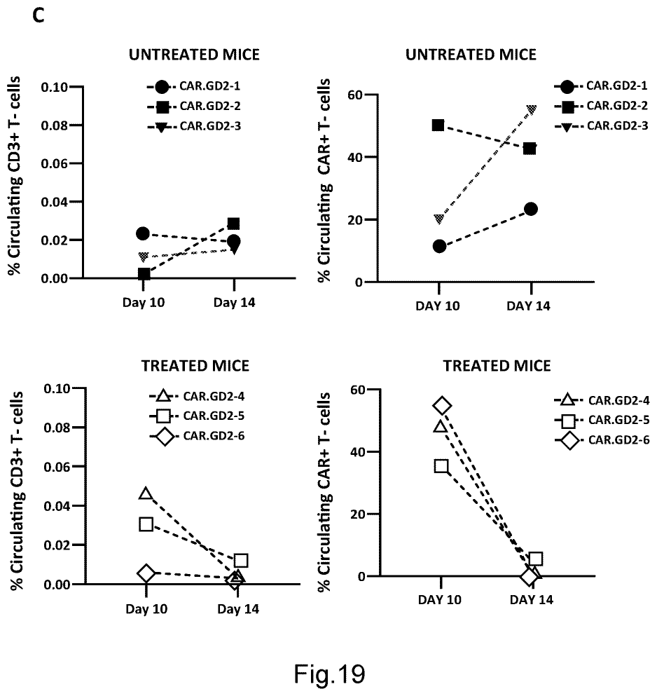

FIG. 19(A-C). Evaluation of the efficacy of AP1903 on circulating CAR.GD2 T-cells in MB in vivo model. (A) Illustration of Experimental design in which MB cells were stereotaxically implanted into the cerebellum of NSG mice. After 30 days from the tumor engraftment, the mice were infused with the effector T cells through iv injection. On day 11, 12 and 13 the mice received three consecutive doses of the dimerizing AP1903 (100 mg/mouse). Flow-cytometry analysis of human T cells circulating in blood of the mice were performed one day before the AP1903 administration and at the end of the experiment (day 14). (B) AP1903 efficiently induced a strong reduction of circulating GD2.CAR-28.4-1BBζ (CAR.GD2) T-cells. Plots are showing an exemplificative CAR.GD2 T-cells in un-treated mice (on the top panel) or treated (at the bottom panel) with AP1903. CAR expression was assessed by the use of anti-idiotype (1A7) (PE) mAb in combination with anti-hCD3 (APC) mAb. (C) Flow cytometric analysis of the human CD3⁺ T cells in MB bearing mice receiving CAR. GD2-T-cells untreated and treated with AP1903 (on the left). On the right is shown the flow cytometric analysis of circulating CD3⁺CAR.GD2⁺ in MB bearing mice receiving CAR.GD2-T-cells, and untreated and treated with AP1903.

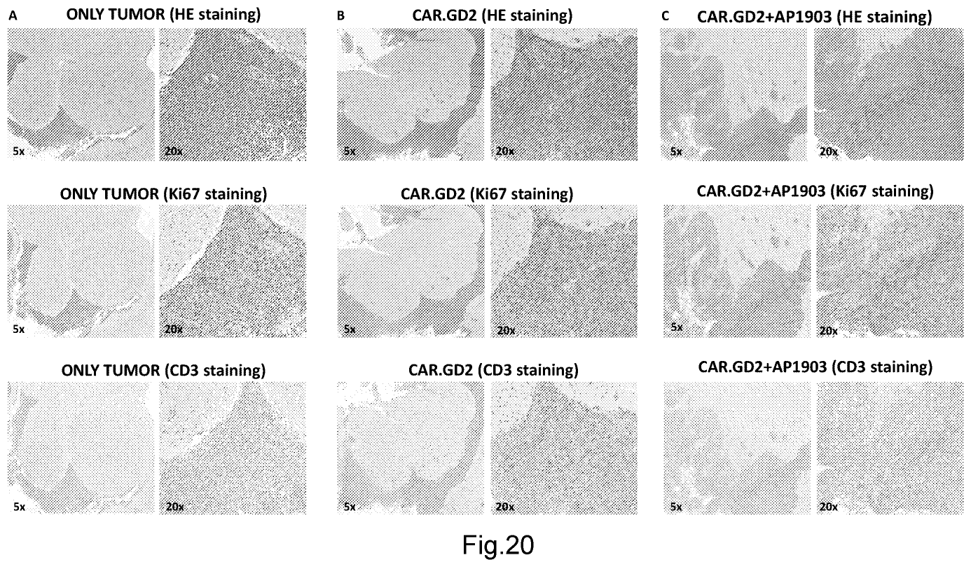

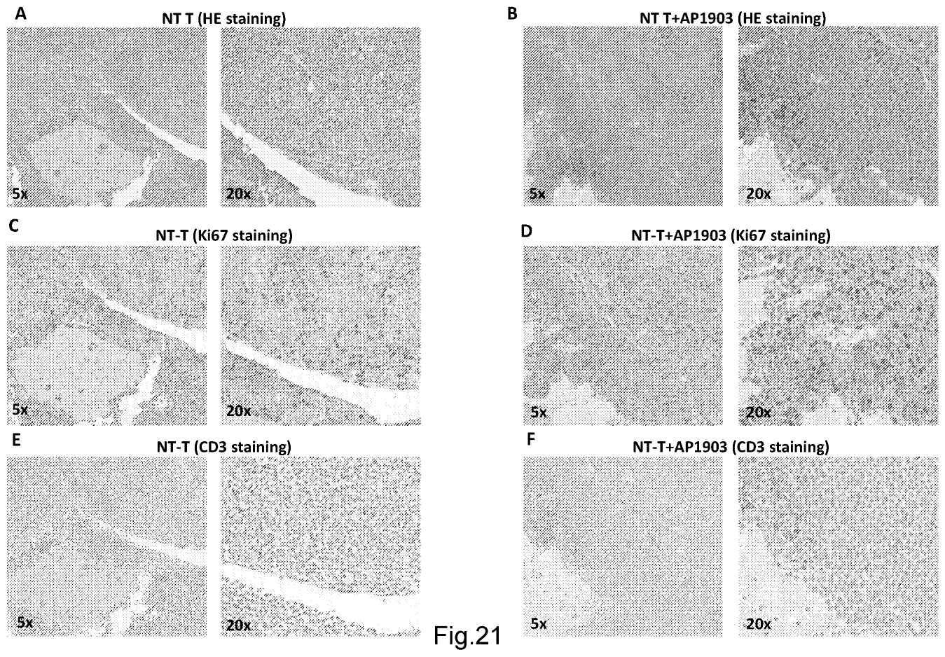

FIG. 20(A-C). Evaluation of the efficacy of AP1903 on GD2.CAR-28.4-1BBζ (CAR.GD2) T-cells infiltrating tumor in MB in vivo model. NSG mice stereotaxically implanted with MB cells. After tumor establishment, mice were infused with effector T-cells. When effector T-cells were detectable in PB, mice received for three consecutive days the dimerizing AP1903 and sacrificed to collect the celeberrum. (A) Exemplificative H&E stained section of control MB tissues into the cerebellum of NSG mice (on the top), representative images of immunohistochemical staining for Ki67 (in the middle) and representative images of human tumor-infiltrating CD3+ cells (bottom panels) are shown. (B) Exemplificative H&E stained section of MB tissues into the cerebellum of NSG mice treated with CAR.GD2 T-cells (on the top), representative images of immunohistochemical staining for Ki67 (in the middle) and representative images of human tumor-infiltrating CD3+ cells (bottom panels) are shown. (C) Exemplificative H&E stained section of MB tissues into the cerebellum of NSG mice treated with CAR.GD2-T cells and AP1903 (on the top), representative images of immunohistochemical staining for Ki67 (in the middle) and representative images of human tumor-infiltrating CD3+ cells (bottom panels) are shown.

FIG. 21(A-B) Representative images of H&E stained section of MB tissues into the cerebellum of NSG mice that received NT T-cells, un-treated (left top panel, A) or treated with AP1903 (right, top panel, B). (C-D) Representative images of immunohistochemical staining for Ki67 of cerebellum of NSG mice treated with NT T-cells; un-treated (left, middle panel, C) or treated with AP1903 (right, middle panel, D) (E-F) Representative images of human tumor-infiltrating CD3+ cells of NSG mice that received NT T-cells, un-treated (left bottom panel, E) or treated with AP1903 (right, bottom panel, F). 5× and 20× magnification was used.

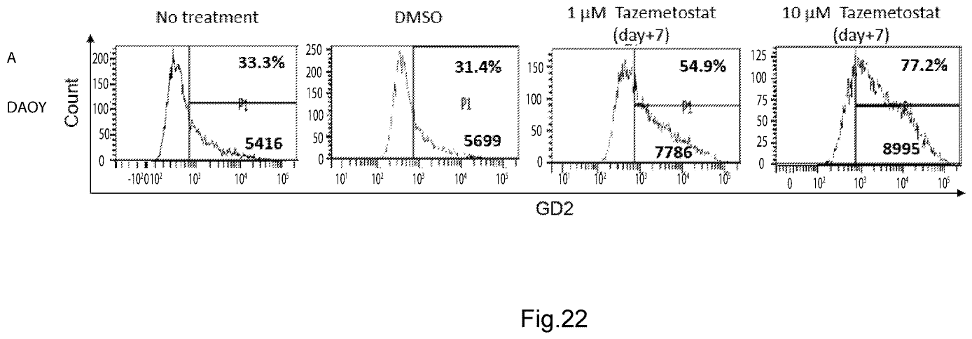

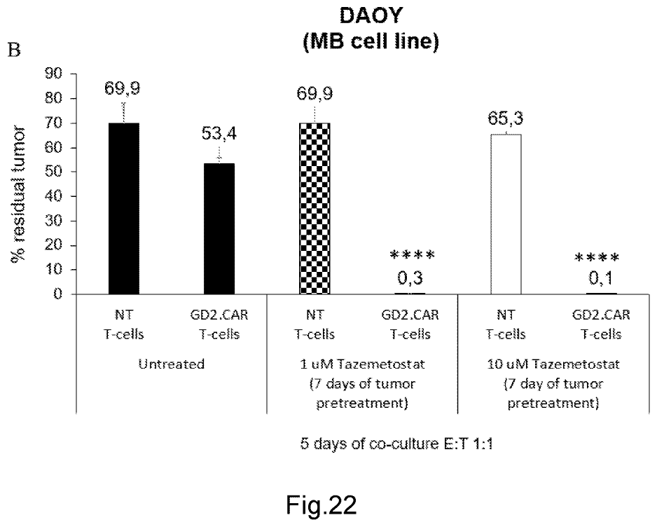

FIG. 22(A-B). Inhibition of EZH2 upregulates surface GD2 expression in DAOY (SHH subgroup) MB cells. (A) Flow-cytometry analyses shows GD2 expression (percentage and MFI) in DAOY (A) MB cell line treated with 1-10 UM of tazemetostat for 7 days. DMSO was included as negative control. (B) Residual tumor was expressed as percentage of remaining tumor cells, after 5 days of co-culture experiments between NT or GD2.CAR-28.4-1BBζ (GD2.CAR) T-cells with MB cell line DAOY, pre-treated for seven days with 1 or 10 UM Tazemetostat, at the E:T ratio of 1:1. Black bars indicate MB cells non treated with the drug tazemetostat; Checkerboard bars indicates MB cells pre-treated with 1 μM of Tazemetostat, and white bars indicates MB cells pre-treated with 10 μM of Tazemetostat. Data from 4 HDs are expressed as average±SD in B; ****p-value=<0.0001.

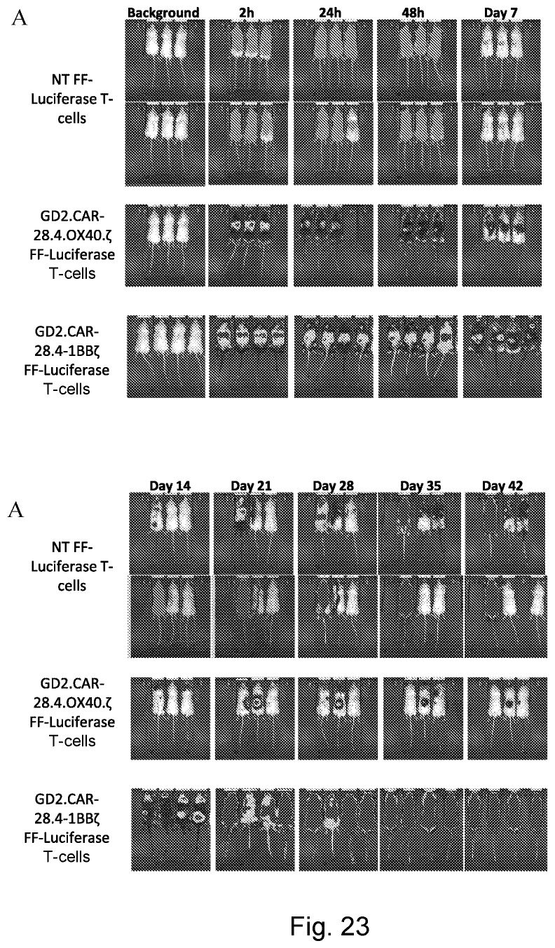

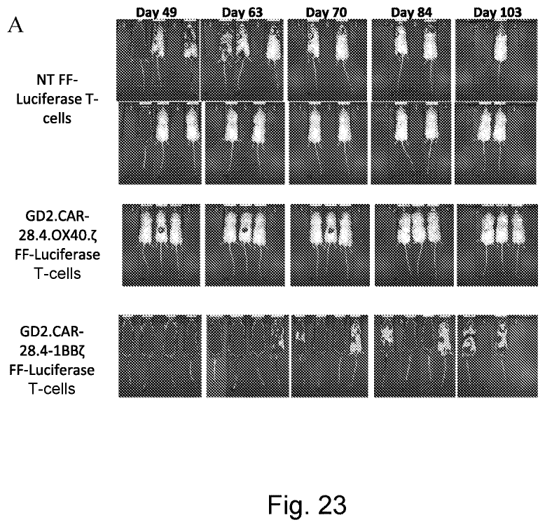

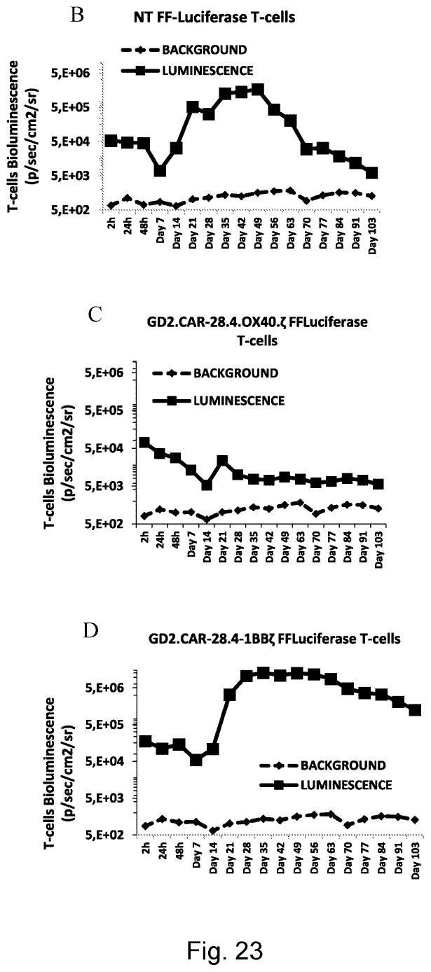

FIG. 23. Orthotopic mouse model of human SHSY5Y-Luc cell line to evaluate long-term persistence of GD2.CAR-28.4-1BBζ (CAR.GD2) T-cells. (A) Effector T-cells FF Luciferase were administered through iv injection. Persistence of effector T-cells (NT FF-Luciferase T-cells, top panels; GD2.CAR-28.4.OX40.Z FF-Luciferase T-cells, middle panel and GD2.CAR-28.4-1BBζ FF-Luciferase T-cells, lower panel) were measured by IVIS bioluminescence system by day 0 up to day 103. (B) GD2.CAR-28.4-1BBζ T-cells persist significantly longer than (C) third-generation GD2-CAR T cells, including CD28 and OX40 as costimulatory domains, and (D) non-transduced (NT) T FF-luciferase T-cells.

### Example 1: In Vitro and In Vivo Evaluation of GD2.CAR Effector Cells According to the Present Invention for the Treatment of GD2+ Solid Tumors Including Brain Tumors with or without Sequential Use of Enhancer of Zeste Homolog 2 (EZH2) Inhibitors

### Methods

### Healthy Donors (HDs) and Samples Patients

Patients' tissues were collected from a cohort of pediatric patients at the Bambino Gesù Children's Hospital (OPBG). Patient tissues and blood samples of both: patient and healthy donors, were collected and evaluated by cytofluorimetric analysis for GD2 expression.

### Cell Lines

The OS cell lines: 143B (ATCC-CRL-8303), MG-63 (ATCC-CRL-1427), U-2 OS (ATCC HTB-96), HOS (ATCC CRL-1543), and SAOS-2 (ATCC HTB-85) cell lines were obtained from LGC Standards S.r.L, Milano (MI), Italy. The embryonal rhabdomyosarcoma (ERMS) cell line RD (ATCC CCL136) and the EWS cell lines: A-673 (ATCCR CRL-1598) and SKES1 (ATCC HTB86) were obtained from LGC Standards S.r.L, Milano (MI), Italy. The ERMS cell line RD were obtained from ATCC (CCL-136), the alveolar rhabdomyosarcoma (ARMS): RH41 and RH30 were obtained from DSMZ.

The melanoma A375 cell line (CRL-1619/ATCC) and lung adenocarcinoma cell line H2228 cell line (CRL-5935/ATCC) were obtained from LGC Standards S.r.L, Milano (MI), Italy.

All cell lines were authenticated by STR analysis in the certificated lab “BMR Genomics s.r.l.”, and tested for mycoplasma every 15 days.

The D283 Med (G3/G4 subgroup) and DAOY (SHH subgroup) MB cell lines were obtained from the American Type Culture Collection (ATCC, USA). DAOY and D283 Med cells were cultured according to the recommended conditions by the supplier (ATCC). Cells were maintained in a humidified atmosphere containing 5% CO2 at 37° C. and were routinely tested for mycoplasma and for surface expression of target antigens. All cell lines have been authenticated by STR analysis in the certificated lab “BMR Genomics s.r.l.” The identity and subgroup of each MB cell line was validated by methylation analysis.

### Retroviral Vectors

The third-generation retroviral vector CAR-GD2.CD28.4-1BBζ (CAR.GD2) including an inducible Caspase 9 (iC9) suicide gene (as safety switch) and the anti GD2 single chain variable fragment (scFv) 14.G2a with two costimulatory domains CD28.4-1BB fused to CD3-ζ chain, was used to transduce T cells⁵¹. An additional retroviral vector carrying an eGFP-Firefly-Luciferase (eGFP-FFLuc) was used to genetically modify the effector T-cells, SHSY5Y or D283 Med and DAOY MB cell lines in D283 Med-GFP-FF-Luc and DAOY-GFP-FF-Luc, respectively, for in vitro and/or in vivo studies.

### Generation of CAR-T Cells

Peripheral blood mononuclear cells (PBMC) were isolated from buffy coats (BC) obtained from healthy donors (HD) (OPBG Hospital, Rome, Italy) who signed a written informed consent, in accordance with rules set by the Institutional Review Board of OPBG (Approval of Ethical Committee N969/2015 prot. N 669 LB). T lymphocytes were activated with OKT3 (1 mg/ml, e-Bioscience Inc; San Diego, CA, USA) and anti-CD28 (1 mg/ml, BD Biosciences, Europe) monoclonal antibody (mAb) in the presence of the combinations of recombinant human interleukin-7 (IL7, 10 ng/ml; R&D; USA) and interleukin-15 (IL15, 5 ng/ml; R&D). Activated T cells were transduced with CAR-GD2.CD28.4-1BBζ retroviral supernatant on day 3 as previously described by Quintarelli et al.⁵¹, to obtain CAR.GD2 T-cells, which were expanded in culture medium, containing 45% RPMI 1640, 45% Click's medium (Sigma-Aldrich, Co.; USA), supplemented with 10% inactivated-FBS North (Gibco, ThermoFischer), and 2 mM GlutaMax (ThermoFischer, Paisley, Scotland) and replenished twice a week.

### Immunophenotype Analysis

Cell surface expression of GD2 was analyzed on tumor fresh cells and peripheral blood (PB) of MB patients using conjugated mouse anti-human GD2-BV421 mAb (BD Biosciences, USA). Cells were also stained with live/dead dye, using 7-Amino-Actinomycin D (7-AAD) staining solution (BD Biosciences, Italy); CD45 APC (BD Biosciences, USA), and the cell population of interest was gated on CD45 negative (CD45-) singlet cell gating.

The expression of CAR. GD2 on T cells was detected using a specific primary anti-idiotype antibody (1A7), followed by a rat anti-mouse secondary antibody (PE), and was evaluated in association with CD3-specific mAb. Treated mice underwent periodical blood collection for FACS analysis. PB of mice, after lysis of red blood cells with lysis buffer (BD), was stained with anti-human CD45 APC (BD Biosciences, USA), anti-human CD3 Pecy7 (BD Biosciences, USA), anti-human CD4 BUV605, anti-human CD8 BUV395 (BD Biosciences, USA) in combination with primary 1A7 anti-idiotype antibody. Cells were incubated with mAbs (30 min at 4° C. in the darkness), then washed in 1× phosphate-buffered saline (1×PBS) and analyzed in a FACS-Fortessa flow cytometer, and data were analyzed by FACSDiva software (Becton Dickinson). For each sample, a minimum of 20,000 events was analyzed.

### Immunohistochemistry (IHC) of Sarcoma Tissues

Sections of formalin-fixed paraffin embedded (FFPE) patient tissues were obtained from surgical samples at the time of biopsy after informed consent. The sections were cut to 2.5 μm thick, dewaxed and rehydrated. The epitope retrieval was performed by incubating the sections at room temperature for 6 minutes with Proteinase K (S3020) (Dako, Glostrup, Denmark). Endogenous peroxidase was blocked with 3% hydrogen peroxide for 10 minutes followed by another blocking step in 5% serum albumin (BSA) for 1 hour RT. Sections were incubated overnight at 4° C. with the monoclonal anti-GD2 antibody (dilution 1:100) (14G2a; cat #LS-C63496, LSBio). Secondary biotinylated antibody (K8024, Dako, Carpinteria, USA) and the peroxidase DAB kit (Dako, Carpinteria, USA) were used to reveal the primary antibodies. Slides were then counterstained with hematoxylin, dehydrated with a series of alcohol solutions (70%-100%), followed by three changes of xylene and mounted with Cytoseal XYL (Thermo Scientific). Analysis was performed using standard microscopy.

### Co-Culture Assay

For in vitro co-culture assay, the un-transduced effector (E) T-cells (NT-T) or CAR.GD2 T-cells (0.1×10⁶ cells/well) were plated with the GD2+ target (T) MB cells (0.1×10⁶ cells/well) in a 24-well plate at the indicated E:T ratio for 5 days. The antitumor effect was evaluated by flow-cytometry assay, assessing either percentage of residual live 7AAD^{negative} GD2^{positive} (7AAD-GD2+) tumor cells or absolute quantization of GD2+ tumor cells remaining in culture after exposure to NT-T or CAR.GD2 T-cells. CountBright™ absolute counting beads (Thermo Fisher Scientific) were used for the absolute quantification of tumor cells.

### In Vitro Anti-Sarcoma Activity

For long-term co-culture experiments, NT and CAR.GD2.28.4-1BBζ (IIICAR.GD2) T lymphocytes were plated at 0.5×10⁶ cells/well in 24-well plates at the indicated Effector:Target (E:T) ratios. Following 6 days of incubation at 37° C., adherent tumor cells and T cells were collected and residual tumor cells and T cells assessed by fluorescence-activated cell-sorting (FACS) analysis based on CD45+/CD3+ cells (Effector T cells) and GFP+GD2+ (sarcoma tumor GD2+ cell line) or CD45-/CD3-(sarcoma tumor GD2 (neg) cell line), respectively. For “stressed” co-cultures assay⁷², tumor cells were added on day 0, 5, 10 and 15 at Effector:Target (E:T) ratio of 1:1. The residual tumor cells and persisting T-cells were analyzed by FACS 5 days after each tumor addition.

### Cytokine Profile

Supernatant were collected at 24 hours from co-cultures medium to measure cytokines release. Cytokines were measured by immunoassay in a microfluidic Simple Plex cartridge ELLA (Biotechne; R&D Systems, Minneapolis). In particular, the following cytokines were investigated: Granzyme B, Interferon gamma (IFN-γ), IL-2 and Tumor Necrosis factor alpha (TNF-α).

Treatment with EZH2 Inhibitors

Tazemetostat is a potent, and selective EZH2 inhibitor (Aurogene, Rome, Italy), dissolved in DMSO or DMSO alone as a control was added at a concentration of 1 or 10 μM to tumor cells. After seven day of incubation at 37° C. and 5% CO2, tumor cells were collected and stained with live/dead dye, using 7-Amino-Actinomycin D (7-AAD) staining solution (BD Biosciences, Italy); CD45 APC (BD Biosciences, USA) and conjugated mouse anti-human GD2-BV421 mAb (BD Biosciences, USA). Cells were incubated with mAbs (30 min at 4° C. in the darkness), then washed in 1× phosphate-buffered saline (1× PBS) and analyzed in a FACS-Fortessa flow cytometer, and data were analyzed by FACSDiva software (Becton Dickinson). For each sample, a minimum of 20,000 events was analyzed.

### Xenograft Sarcoma Mouse Model for In Vivo Studies

Xenograft studies were performed using NSG (NOD.Cg-Prkdcscid II2rgtm1Wjl/SzJ; from Charles River) mice 6 to 8 weeks of age. To investigate the in vivo antitumor activity of IIICAR.GD2 T-cells on systemic rhabdomyosarcoma model, 0.5×106 GD2+ RD-GFP-FF-Luc cells were Intravenous injected (i.v.).

To investigate the in vivo antitumor activity of CAR. GD2 T cells on orthotopic (o.t.) model of OS, NGS mice were inoculated in the tibia with 0.5×106 GD2+ 143B-GFP-FF-Luc or 0.2×106 GD2+U-20S-GFP-FF-Luc. Tumors were injected with matrigel diluted 1:1 in PBS 1×. After tumor engraftment, the mice received only one i.v. injection of effector T cells (10×106/mouse). To investigate which human sarcoma lines modulate mouse MDSC cell expansion, the OS cell lines: 143B, SAOS-2 and U-20S were orthotopically inoculated in the right tibia of NSG mice. For the ERMS model, RD cell line was orthotopically inoculated in the right paw muscle of NSG mice.

Tumor growth was evaluated using IVIS imaging system (PerkinElmer, USA), Briefly, a constant region of interest was drawn over the mouse and the intensity of the signal measured, every week, as total photon/see/cm2/sr (p/s/cm2/sr), as previously described⁷³. The circulating human T cells were evaluated periodically in mice peripheral blood. Mice were maintained in the animal facility at Plaisant Castel Romano (Rome, Italy). All in vivo experiments were in compliance with the ethical international, EU and national requirements and were approved by the Italian Health Ministry (N°88/2016-PR).

### Xenograft Medulloblastoma Mouse Model for In Vivo Studies

NOD/SCID IL-2Rγnull (NSG) xenograft mice were purchased from Charles River and maintained in the Plaisant Castel Romano facility in Rome. Mice experiments were conducted in compliance with the ethical international, EU and national requirements and were approved by the Italian Health Ministry (n° 765/2021-PR). For the orthotopic in vivo model, 5-week-old female NSG mice were anesthetized by intraperitoneal injection of ketamine (10 mg/kg) and xylazine (100 mg/kg). The posterior cranial region was shaved and placed in a stereotaxic head frame. D283 MED.GFP-FF.Luc cells (2×10⁵/each mouse) were stereotaxically implanted into the cerebellum at an infusion rate of 1 μL/minute by using the following coordinates, according to the atlas of Franklin and Paxinos: 6.6 mm posterior to the bregma; 1 mm lateral to the midline; and 2 mm ventral from the surface of the skull. After injection, the cannula was kept in place for about 5 minutes for equilibration of pressures within the cranial vault. The skin was closed over the cranioplastic assembly using metallic clips. After 3 days of tumor engraftment, mice were randomly divided in two groups, intravenously (i.v.) injected with 10×10⁶ control NT T-cells or CAR.GD2 T-cells and subjected to weekly bioluminescence imaging (IVIS System, Perkin Elmer, USA) until 45 days (end of the experiment). Signal quantitation of photons/second was performed as previously described^{73, 74}. Moreover, a MB Patient Derived Xenograft (PDX-MB) mouse model was established by stereotaxic implantation of Med-411FH mCherry/Luciferase cells (2×10⁵/each mouse). Fourteen days following tumor implantation, mice were i.v. treated with NT T or CAR.GD2 T-cells (10×10⁶/each mouse) and monitored for tumor growth by IVIS imaging until 60 days (end of the experiment). All mice were sacrificed according to protocol when moribund or upon the development of hind-limb paralysis. In both above mentioned in vivo experiments, the expansion of effector cells was monitored by blood bleedings and analyzed by flow BD LSR Fortessa X-20 cytometry. Data were analyzed using the FACSDiva software (BD Biosciences, Italy).

### In Vivo Study of the Activation of Suicide Gene by Dimerizing Drug AP1903 Infusion

The in vivo activity of AP1903 on apoptosis induction of CAR. GD2 T-cells was assessed in in vivo PDX-MB mouse model. Briefly, 2×10⁵ Med-411FH mCherry/Luciferase were orthotopic infused in 5-week-old female NSG mice. After tumor engraftment, confirmed by bioluminescence monitoring, mice received an intravenous injection (i.v.) of 10×10⁶ of NT T or genetically modified CAR.GD2 T cells. Ten days later, when the presence of circulating T cells was confirmed by flow cytometric analysis, mice were randomly divided in four groups and treated or not treated by intraperitoneal (i.p.) infusion with AP1903 (100 mg/mouse) on day +11, day +12 and day +13 (NT-T no AP1903, NT-T AP1903+ or CAR. GD2-T no AP1903, CAR.GD2-T AP1903+). The residual of effector cells was evaluated 24 hours after the last AP1903 treatment by flow cytometric analysis in the peripheral blood or histologic and IHC analysis in brain/tumor tissue. Animals were sacrificed and brains were fixed in 4% formaldehyde in 0.1 mol/L phosphate buffer (pH 7.2) and paraffin embedded. AP1903 (cat. No. 6130, Biotechne-TOCRIS).

### Haematoxylin/Eosin and Immunohistopathological (IHC) Analysis on Mouse Tissues

The histopathologic analysis was performed as previously reported⁷⁴. Briefly, after Haematoxylin/Eosin staining for IHC analysis, the sections were cut to 2.5 μm thick, dewaxed, and rehydrated. Epitope retrieval was performed by boiling the slides with EDTA (pH 9). Endogenous peroxidase was blocked with 3% hydrogen peroxide for 10 minutes followed by another blocking step in 5% serum albumin (BSA) for 1-hour RT. The sections were then incubated with anti-CD3 antibody (1:100, DAKO) or anti-Ki67 (1:100, Abcam) at 4° C. overnight. Secondary biotinylated antibody (K8024, Dako, Carpinteria, USA) and the peroxidase DAB kit (Dako, Carpinteria, USA) were used to reveal the primary antibodies. Slides were then counterstained with hematoxylin, dehydrated with a series of alcohol solutions (70%-100%), followed by three changes of Diasolv and mounted with Diamount (Diapath). Analysis was performed using standard microscopy.

### IHC Analysis on Human Tissues

All samples of human tissue in this study were collected at the Bambino Gesù Children's Hospital in Rome with the approval of the Institutional Review Board. This clinical MB specimens were examined and diagnosed by pathologists. Sections of formalin-fixed paraffin embedded (FFPE) patient tissues were obtained from surgical samples at the time of resection after informed consent. The sections were cut to 2.5 μm thick, dewaxed, and rehydrated. The epitope retrieval was performed by incubating the sections at room temperature for 6 minutes with Proteinase K (S3020) (Dako, Glostrup, Denmark). Endogenous peroxidase was blocked with 3% hydrogen peroxide for 10 minutes followed by another blocking step in 5% serum albumin (BSA) for 1-hour at room temperature (RT). Sections were incubated overnight at 4° C. with the monoclonal anti-GD2 antibody (dilution 1:100) (14G2a; cat #LS-C63496, LSBio). Secondary biotinylated antibody (K8024, Dako, Carpinteria, USA) and the peroxidase DAB kit (Dako, Carpinteria, USA) were used to reveal the primary antibodies. Slides were then counterstained with hematoxylin, dehydrated with a series of alcohol solutions (70%-100%), followed by three changes of Diasolv, and mounted with Diamount (Diapath). Analysis was performed using standard microscopy. The data shown in Table 4 were generated based on the scores of IHC antibody quantified as follow: 3+, positive signals in >70% tumor cells; 2+, positive signals in >40 to 69% tumor cells; 1+, positive signals in >5% to 39% tumors cells; 0%, low or no positive signals in <5% tumor cells. The analyses were assessed by an experienced pathologist.

### Statistical Analysis

All data are presented as means±SD. Student t-test or ordinary two-sided one-way ANOVA (Tukey's multiple comparisons test) was used, where appropriate, for comparing differences between groups. The Kaplan-Meier method was used to estimate disease-free survival (DFS) probabilities; differences between groups were compared with the log-rank test. Mice were matched based on the tumor signal for control and treated groups. Graph generation and statistical analyses were performed using Prism version 8.0d software (GraphPad). In all cases, a p-value of ≤0.05 was considered significant. Exact p-values are given whenever suitable. Wilcoxon or Mann-Whitney nonparametric tests were employed. Statistical significances (p value: *p<0.05, **p≤0.01, ***p≤0.001, ****p≤0.0001) are indicated.

### Results

The transduction efficiency of GD2.CAR-28.4-1BBζ T-cells (GD2.CAR T-cells), transduced with the third generation SFG.iC9.2A.CAR-GD2 (14g2a) CD8tm.CD28.41BBζ (here after iC9.2A.GD2.CAR-28.4-1BB.2) retroviral vector (FIG. 1A), is equal to 65.1%+9.7% vs Non-transduced (NT) T cells, 3.8%+2.3%, p≤0.0001 (FIG. 1B-C). GD2.CAR T-cells growth with the same kinetics of control NT T cells (FIG. 1D). iC9.2A.GD2.CAR-28.4-1BB.ζ retroviral vector transduce with high efficiency also (CD3-CD56+) NK cells, expanded in feeder free media. At day+5 from transduction, the transduction efficiency of NK with GD2.CAR T-cells is equal to 57.77%±11, 12%, and remain stable up to day +25 from transduction: 53, 60%±11.66%, p=0.104 (FIG. 1E).

### Expression of GD2 in Human Sarcoma Cell Lines.

The expression of disialoganglioside GD2 in eleven sarcoma lines was evaluated: specifically, five Osteosarcoma (OS), one embryonal rhabdomyosarcoma (ERMS) cell line: RD, three Alveolar Rhabdomyosarcoma (ARMS) and two Ewing Sarcoma (EW) cell lines, by flow cytometry analysis (FACS). Three OS cell lines 143B, MG-63 and U-20S and one ERMS (RD) cell line showed the highest expression of GD2 (both as a percentage and Median Fluorescence Intensity (MFI)). The OS cell lines: HOS and SAOS-2; the ARMS: RH4, RH41 and RH30; and the EW cell lines: A673 and SKES1 showed the lower expression of GD2 antigens. (FIG. 2A-B). Several tumour cell lines show high expression of GD2, such as melanoma cell line A375 (FIG. 2C-D), lung adenocarcinoma cell line H2228 (FIGS. 2E-F) and very rare tumour as desmoplastic small round cell tumors (DSRCT) (FIG. 2G).

GD2.CAR-28.4-1BBζ (GD.CAR) T-Cells Significantly Control Tumor Growth of Sarcoma Cells with Both High and Low GD2 Expression of GD2

The long-term (5 days) co-culture experiments, at the Effector:Target (E:T) ratio 1:1 (FIG. 3A-F), provide evidences that GD2.CAR-28.4-1BBζ T-cells significantly control the growth of tumor cells, as compared to NT T-cells, having high GD2 expression, such as the OS cell lines: MG63 cells (1.68±2.36% vs 76.88±2.54% of residual tumor cells respectively, p<0.0001; FIG. 3A); 143B cells (1.9±7.2% vs 71.2±9.6% of residual tumor cells respectively, p<0.0001; FIG. 3B and the U-20S cells (11.3±14.1% vs 74.6±17.9% of residual tumor cells respectively, p=0.0007; FIG. 3C; the ERMS RD cell line (4.1%±3.9% vs 74.5%±14.6% of residual tumor cells, p<0.0001, FIG. 3E); Unexpectedly, (compared to NT T-cells) GD2.CAR-28.4-1BBζ T-cells significantly (although partially) control tumor growth also of sarcoma cell lines with lower expression of GD2, such as the OS cell line HOS (22.9±10.3% vs 60.8±13.5% of residual tumor cells respectively, p=0.0003; FIG. 3D); of the ARMS cell line RH4 (GD2=33.6±9.9% vs 70.5±8.6% of residual tumor cells, p=0.0003, FIG. 3F); of the ARMS RH41 (42.9+6.6% vs 80.0+10.5% of residual tumor cells respectively, p=0.004; FIG. 3G) and Ewing sarcoma A-673 cell line (56.5±13.2% vs 81.2±5.4% of residual tumor cells, p=0.0015, FIG. 3H).

### GD2.CAR-28.4-1BBζ (GD.CAR) T-Cells Significantly Control Tumor Growth of Aggressive Adult Tumors.

GD2.CAR-28.4-1BBζ T-cells significantly control also the growth of adult aggressive tumours, such as melanoma cell line A375 (GD2=3.2±0.8% vs 90.40±1.3% of residual tumour cells, p=0.0002, FIG. 3I) and adenocarcinoma cell line H2228 (GD2=4.4±1.68% vs 80.57±4.2% of residual tumor cells, p=0.0006, FIG. 3J).

Inhibition of EZH2 in RMS Cell Lines Up-Reregulates GD2 Expression in Both Ewing Sarcoma and ARMS Sarcoma Cell Lines and Increase their Sensibility to GD2.CAR T-Cell Killing Activity.

As expected EZH2 inhibition induce GD2 up-regulation in EwS A-673 (FIG. 4).

According to the invention, the aim was to investigate whether EZH2 inhibition can induce GD2 up-regulation also in RMS and OS cell lines.

The ARMS FP-RMS cell lines (RH4 and RH41 and RH30, FIGS. 5A-C) and OS cell line HOS (FIG. 6) were cultured with 1-10 μM of Tazemetostat for seven days. Unexpectedly drug treatment significantly increases GD2 expression in two out of three ARMS FP-RMS cell lines: RH4 (FIG. 5A) and RH41 (FIG. 5B), and in OS cell line HOS (FIG. 6). Therefore, seven days EZH2 pre-treatment sensitizes the EwS A-673 cells to effective cytolysis by GD2.CAR-28.4-1BBζ T-cells (FIG. 6A) and more important for the first time it was shown that, beside EwS (FIG. 7A), EZH2 pre-treatment can sensitizes the ARMS RH4 (FIG. 7B) RH41 (FIG. 7C) and HOS cell lines (FIG. 7D) to effective cytolysis by GD2.CAR-28.4-1BBζ T-cells (FIG. 7A-D). An expert in the field looking the literature²¹ would have been discouraged from using this drug (Tazemetostat) to positively modulate GD2 on OS cell line. So, unexpectedly, it was shown that the use of EZH2 inhibitor positively modulate GD2 expression on both Rhabdomyosarcoma cell lines and OS cells. More importantly, Tazemetostat sensitizes these tumor cell lines to effective cytolysis by GD2.CAR-28.4-1BBζ T-cells.

### Evaluation of Long-Term Efficacy of GD2.CAR-28.4-1BBζ T-Cells in Embryonal RMS Mice Model.

It was next assessed whether GD2.CAR-28.4-1BBζ T-cells were able to develop a potent antitumor activity in aggressive metastatic embryonal ERMS RD-GFP-FF-Luc in vivo model, as shown by the cartoon in FIG. 8A. The bioluminescence in RMS tumor-bearing mice, treated with NT T-cells, rapidly increased up to three logs in less than 50 days (FIGS. 8B-D) and mice either died or were sacrificed due to morbidity. The macroscopic analysis in sacrificed mice showed large tumor masses with metastasis located preferentially in the kidney and liver.

Median of survival of RMS-tumor-bearing mice treated with GD2.CAR-28.4-1BBζ T-cells was significantly longer (69.5 days) compared to mice treated with NT (50.0 days) (FIG. 8E; p=0.012). Although circulating T cells in mice treated with NT T-cells were detectable during mice follow-up, they rapidly decreased (from 21.99%+12.16% at day+21 to 12.92%±5.99% at day+47 (FIG. 8F), and no tumour control was observed. In contrast in mice treated with GD2.CAR-28.4-1BBζ T-cells, a significant increase of circulating T cells from 13.44%±11.60% at day+21 to 25.48%±10.65% (p=0.038) at day+47 (p=0.04), and long-lasting persistence of T cell up to 75 days (9.20%±9.80%) were observed. Although the percentage of “CAR expression” in circulating GD2.CAR-28.4-1BBζ T-cells decrease in the first 50 days from 60.75%±16.54% to 28.08%±16.54% (p=0.0001), it remains stable up to day +75 (31.07%±14.76%; p=ns) (FIG. 8G). During tumor eradication, a significant reduction of CD4+CAR+ T-cells (FIG. 8H) with a significant enrichment of CD8+CAR+ T-cells (FIG. 8I) was observed.

Note that there are no data in the literature regarding effective GD2.CAR T cell control of soft sarcomas such as rhabdomyosarcomas.

Improved Survivor of OS Orthotopic Mice Treated with CAR.GD2.28.4-1BBζ-T Cells

For further supporting our in vitro/vivo results, in vivo efficacy, persistence of GD2.CAR-28.4-1BBζ T-cells in 143B-GFP-FF-Luc orthotopic OS mouse model were then evaluated, as described by the cartoon (FIG. 9A). GD2.CAR-28.4-1BBζ (GD2.CAR) T-cells significantly slowed down the tumor growth of primary tumor (FIG. 9B-D), increasing significantly the overall survival of the mice treated with CAR T-cells respect to NT T-cells (FIG. 9E) (46 days for mice treated with GD2.CAR-28.4-1BBζ T-cells vs 39 days of mice treated with NT T-cells respectively, p=0.005). Interesting, a great expansion of human T cells only in mice treated with GD2.CAR-28.4-1BBζ T-cells (FIG. 9F) was observed. The percentage of circulating CAR.GD2-T cells increase in the first month from 46.32%±19.81% to 61.72%±23.78% (day+33) and remained stable up to day 44 (62.80%±17.54%) (FIG. 9E) with an equal distribution between CD4+CAR+ T-cells (53.8%±17.2%) (FIG. 9H) and CD8+CAR T-cells (43.0%±15.4%) (FIG. 9I).

As stated above, GD2.CAR-28.4-1BBζ T-cells according to the invention show to be superior with respect to GD2-CAR incorporating the 14g2a-scFv with the CD28, OX40, and CD3zeta signalling domains (14g2a.CD28.OX40.ζ) described recently by Adrienne H Long et al⁶⁷. This can be deduced by comparing the overall survival of OS mice treated with the GD2.CAR of the invention (FIG. 8E) to that reported in the literature by Adrienne H Long et al⁶⁷ (see page 873, FIG. 3B on Cancer Immunology Res; 4 (10) October 2016).

### Expression of GD2 in Neuroblastoma (NBL) and Retinoblastoma Cell Lines.

Several tumor cell lines express high level of GD2 antigen and then can be targeted by GD2.CAR-28.4-1BBζ (GD2.CAR) T-cells. Specifically, all neuroblastoma (NBL) cell lines show high expression of the diasalogangloside GD2 (FIG. 10A). These data were confirmed on primary tumor derived from patients with diagnosis of Neuroblastoma (NBL) (FIG. 10B). The peripheral blood derived from NBL patients were used as negative control.

High expression of GD2 was also found in all retinoblastoma tumor tissues (FIG. 11).

### Expression of GD2 in Medulloblastoma

It was evaluated whether GD2 could be selected also as a MB tumor-associated antigen and could be targeted using CAR.GD2 T-cells. Moreover, since the use of CAR.GD2 T-cells could induce potential side effects, such as cytokine release syndrome⁷⁵ or neurological toxicities⁷⁵, it was also evaluated and demonstrated the efficacy of inducible caspase 9 (iC9) suicide gene activation to eliminate circulating and tumor-infiltrating CAR.GD2 T-cells, infused into the orthotopic MB mouse model.

### GD2 Expression in Primary MB Biopsies and MB Cell Models

To investigate whether GD2 could be a suitable target for CAR.GD2 T-cell therapeutic approach for MB patients, GD2 expression was evaluated on both tumor biopsies and PB of forty-one patients affected by MB and clinically followed in our department. The flow cytometric analysis performed on fresh tumor tissues of patients prospectively enrolled in the study, showed GD2 positivity in 75.6% of the analysed samples, with an average of 28.84%±33.78% GD2 positive cells (in the gate of CD45-cells). Peripheral blood mononuclear cells of the same MB patients were analysed in parallel, as an internal negative control (% of GD2 positive cells being equal to 0.43%±0.90%; p<0.0001) (FIG. 12A). The median fluorescence intensity (MFI) for GD2 expression was significantly higher on MB tissue biopsies (16165±20161) than on cells from PB samples (1026±1021, p<0.0001) (FIG. 12B). Interestingly, when MB samples were stratified in genetic subgroups, GD2 positivity was highly observed in SHH patients (9 out of 9, 100%), in G4 patients (15 patients out of 16, 93%), in G3 patients (5 out of 7, 71.43%), whereas WNT subgroup was characterized by a low percentage of patients with GD2 positivity (2 out of 9, 22%) and a low frequency in the tumor of GD2+ cells (1.61%±4.03% of CD45-analyzed cells). (FIG. 12C). Nevertheless, patients were also stratified based on histologically defined subgroups, and GD2 positivity was observed in 76% (19 out of 25) Classic MB patients, 50% (4 out of 8) large cell/anaplastic (LCA) MB patients, three out of three (100%) of MBEN MB subgroups and five out of five (100%) of desmoplastic/nodular (D/N) MB subgroups patients (FIG. 12E). Due to the high variability of GD2 expression (both in terms of percentage and MFI) between MB samples, GD2 positivity was no significantly different between the considered histological subgroups (FIG. 12D-F). FIG. 13 show four representative FACS analysis of four different MB tumours (SHH, G3, G4 and WNT). GD2 expression was also confirmed by IHC analysis on 18 tissue biopsies (9 G3, 4 G4 and 5 SHH patients) (Table 4).

TABLE 4

| | | | GD2 signal | | | |
|---|---|---|---|---|---|---|
| | | | Cytoplasmatic | | Nuclear | |
| Patients | Diagnosis | Molecular Classification | Pattern | Intensity | Pattern | Intensity |
| 1 | Medulloblastoma | Group 3 | No signal | − | No signal | − |
| 2 | Medulloblastoma | Group 3 | No signal | − | No signal | − |
| 3 | Medulloblastoma | Group 3 | Focal | + | No signal | − |
| 4 | Medulloblastoma | Group 3 | Diffuse | ++ | Diffuse | ++ |
| 5 | Medulloblastoma | Group 3 | No signal | − | No signal | − |
| 6 | Medulloblastoma | Group 3 | Focal | + | Focal | + |
| 7 | Medulloblastoma | Group 3 | Diffuse | ++ | Diffuse | ++ |
| 8 | Medulloblastoma | Group 3 | Focal | + | Focal | + |
| 9 | Medulloblastoma | Group 3 | Focal | + | No signal | − |
| 10 | Medulloblastoma | Group 4 | Diffuse | +++ | No signal | − |
| 11 | Medulloblastoma | Group 4 | No signal | − | Diffuse | ++ |
| 12 | Medulloblastoma | Group 4 | Diffuse | + | No signal | − |
| 13 | Medulloblastoma | Group 4 | Diffuse | + | No signal | − |
| 14 | Medulloblastoma | SHH | No signal | − | No signal | − |
| 15 | Medulloblastoma | SHH | Focal | ++ | No signal | − |
| 16 | Medulloblastoma | SHH | Diffuse | + | No signal | − |
| 17 | Medulloblastoma | SHH | No signal | − | No signal | − |
| 18 | Medulloblastoma | SHH | Diffuse | + | Diffuse | + |

Similar to neuroblastoma, lung cancers⁵³, breast cancer⁷⁶ and gliomas⁶³, the majority of IHC staining in MB tumors was found variable and within the cytoplasm (Table 4). Finally, the expression of disialoganglioside GD2 was evaluated in two MB cell lines and one PDX (Patient derived xenografts) cell line, that will be employed in the immunotherapy model according to the present invention. In particular, D283 Med (subgroup G3/G4) cell line show the highest GD2 expression (100%±0.0% and MFI equal to 42354±2119), whereas the DAOY (SHH) cell line show 30.40%±2.1% GD2+ cells with an MFI of 15589±648). (FIG. 14A-C).

### CAR.GD2 T-Cells Exert Effective Antitumor Activity Toward Human GD2+MB Cell Lines

Since a significant relevant expression of GD2 antigen was found in most tumor tissues from MB patients, the in vitro cytotoxic activity of the 3rd generation CAR-GD2.CD28.4-1BBζ (CAR.GD2) T-cells according to the invention was tested against two MB human cell lines D283 Med (subgroup G3/G4) and DAOY (SHH) cells.

In in vitro long-term co-culture assays, NT T-cells and CAR.GD2 T-cells were incubated for 5 days with D283 Med or DAOY cell lines at the effector:target (E:T) ratio of 1:1.

CAR.GD2-T cells were able to significantly eradicate D283 cell line (p<0.0001) (FIG. 15A) compared to NT T-cells. Moreover, cytokine production strongly correlated with the higher anti-MB killing activity of CAR. GD2 T-cells (FIG. 15B). A significantly higher amount of activation cytokines as Granzyme B, IFN-γ, IL2 and TNF-α, were produced by CAR. GD2 T-cells co-cultured with GD2+D283 Med (FIG. 15B).

By contrast when CAR.GD2-T cells were co-cultured with DAOY cells, characterized by a lower percentage of GD2+ cells as well as by a reduced GD2 MFI (FIG. 14A-C) respect to D283 MED cells, a suboptimal, although significant respect to non-transduced (NT) T cells, in vitro tumor control was observed, as percentage of residual tumor (equal to 27.88%±12.88% vs 51.36%±13.24%, respectively, p=0.03) (FIG. 15C) and in parallel, a dismal production of activating cytokines (FIG. 15D) was observed. The residual DAOY cells resistant to the elimination by CAR.GD2 T-cells were then characterized by flow-cytometry, and it was observed that the spared DAOY cells were mostly GD2 negative or having a very low GD2 MFI (FIG. 16A-B).

### CAR.GD2-T Cells Exert Antitumor Activity in Xenogeneic Mouse Model of MB

It was then assessed whether CAR.GD2 T-cells were able to develop a significant antitumor activity in the orthotopic MB NSG mouse model of D283 Med (subgroup G3/G4) cell line, genetically modified with GFP-firefly luciferase (D283 Med-GFP-FF-Luc) for the in vivo bioluminescence monitoring. MB cell line was intracranially implanted and bioluminescent imaging (BLI) was used to monitor tumor growth overtime in NSG mouse model. Three days after tumor engraftment, mice were intravenously treated with either NT T-cells or CAR.GD2 T-cells on day 0 (FIG. 17A). As expected, tumor bioluminescence rapidly increased up to three logs in less than one month in mice treated with NT T-cells (FIGS. 17B-C) and mice either died or were sacrificed due to morbidity. The cohort of mice receiving CAR.GD2-T cells showed the complete tumor eradication after 38 days from infusion (FIG. 17B-C) with a significant prolongation of DFS compared to NT-T mice group. As shown in FIG. 17D, the 80% of mice treated with CAR.GD2-T cells showed an improved survival at 90 days (end of the experiment) compared to NT-T cohort (p=0.03). Notably, the in vivo anti-tumor efficacy of CAR.GD2-T cells was correlated with a long persistence (up to 45 days) of circulating effector cells (CD45+/CD3+) in PB (FIG. 17E). More important, the percentage of circulating CAR+ T cells remained stable for 45 days (FIG. 17F). The dynamic evolution of both CD4+ and CD8+ T cells was also evaluated in PB of treated mice and a slight increase of CD8+ T cells in both NT and CAR. GD2 T-cell population was observed (FIG. 18A-C).

Although it was reported that the activation of suicide gene iC9, which was included in the construct of the invention, led to prompt apoptosis of CAR-T cells both in in vitro and in vivo only in extra cranic tumor, specifically in neuroblastoma models⁵¹, it had never been evaluated whether the systemic administration of AP1903 could induce a rapid elimination of for brain/MB-infiltrating CAR T-cells. To this aim, an orthotopic PDX mouse model implanting MB tumor cells was developed. After 10 days of intravenously infusion of either NT T or CAR.GD2 T-cells, three mice in each cohort, received the dimerizing AP1903 intraperitoneally for three consecutive days (FIG. 19A). As shown in FIG. 19B-C, AP1903 administration allowed a significant reduction of circulating CAR.GD2-T cells compared to untreated CAR.GD2 T-cells. Moreover, in a dedicated experiment, the murine brain bearing the MB tumors was explanted after 44 days from the NT/CAR.GD2 T-cell infusion, to characterize the tissue for the human T cell infiltration (IHC vs hCD3). The ability of CAR. GD2-T cells to migrate across the blood-brain-barrier (BBB) and localize into the tumor (FIG. 20, panel B) was demonstrated, whereas very negligible number of tumor-infiltrating human T cells were observed into the brain of mice receiving NT T-cells (FIG. 21). Most importantly, anti-hCD3 IHC analysis on cerebella slides of mice infused with CAR.GD2-T cells that received AP1903 administration clearly revealed the effectiveness of AP1903 to cross the BBB and to eliminate tumor-infiltrating CD3+ cells (FIG. 20B-C).

### Inhibition of EZH2 in MB Cell Lines Up-Reregulates GD2 Expression in MB Tumor

It was investigated whether EZH2 inhibition can induce GD2 up-regulation also in Medulloblastoma cell line with low expression Of GD2. The DAOY (SHH subgroup) MB cell line was cultured with 1-10 UM of Tazemetostat for seven days. Unexpectedly drug treatment significantly increases GD2 expression in DAOY cell line (FIG. 22A) in percentage (from 33.3% to 77.2%) and Median Fluorescence Intensity (MFI) (from 5416 to 8995). Therefore, seven days EZH2 pre-treatment sensitizes the MB DAOY cells to effective cytolysis by GD2.CAR-28.4-1BBζ T-cells (FIG. 22B).

### Orthotopic Mouse Model of Human SHSY5Y-Luc Cell Line to Evaluate Long-Term Persistence of GD2.CAR-28.4-1BBζ (CAR.GD2) T-Cells.

In xenograft neuroblastoma mouse model (SHSY5Y), the long-term persistence of infused effector T-cells genetically modified to express FF-Luciferase (23A) was evaluated. NT FF-Luciferase T-cells, GD2.CAR-28.4.OX40.7 FF-Luciferase T-cells and GD2.CAR-28.4-1BBζ FF-Luciferase T-cells were administered through iv injection, and T-cells bioluminescence was monitored for 102 days. GD2.CAR-28.4-1BBζ T-cells (23B) persist significantly longer than (23C) third-generation GD2-CAR T cells, including CD28 and OX40 as costimulatory domains, and (23D) non-transduced (NT) T FF-luciferase T-cells. In the field of CAR T cell-based therapies, recent pre-clinical and clinical studies have shown promising results for the treatment of cancer. Although clinical trials based on anti-CD19.CAR T-cells achieved unprecedented results for the treatment of hematological neoplasia, there is limited documented information about antitumor activity against solid neoplasms. In particular, the identification of a suitable target antigen, highly expressed in solid tumors and with a restricted expression in normal tissues, represents a limitation to overcome. The disialoganglioside GD2 is the antigen over expressed on the cell surface of a wide spectrum of human cancers, such as neuroblastoma, melanomas, retinoblastomas, Ewing sarcomas, small cell lung cancer, gliomas, osteosarcomas, and soft tissue sarcoma², with very low expression in healthy tissues. Despite the function of GD2 is not fully understood, it is clearly known its involvement in tumor development and malignant phenotypes through the increase of cell proliferation, growth, motility, migration, adhesion, and invasion^{6, 54}. Recently it has been demonstrated that diffuse midline gliomas (DMG) H3K27M-mutant, including diffuse intrinsic pontine glioma (DIPG), unequivocally express GD2 antigen^{52, 77}. Notably, recently, it has been reported the safety of CAR.GD2 T-cell infusion in patients with H3K27M-mutated DIPG or spinal cord DMG, showing that toxicity was largely related to the peculiar location of the tumor and anyway, was reversible with intensive supportive care⁵². Here it is reported for the first time that GD2 antigen is expressed in majority large proportion of MB cases at diagnosis. Importantly, SHH and G3/G4 subgroups showed the highest GD2 expression, leading us to leverage GD2 to a suitable immunotherapy target for MB patients with the most aggressive and worst prognosis.

These insights provide support for the rationale to develop an adoptive cell therapy targeting GD2 in pediatric MB. Above all, considering that despite standard intensive care, most of patients have a poor prognosis, CAR T-cells redirected against GD2 antigen could represent a potential curative approach. First, the significant anti-tumor activity of CAR. GD2 T-cells was proved in MB in vitro and in vivo models. In particular, CAR.GD2 T-cells mediated remarkable killing activity against GD2+ MB cell lines and a MB PDX cell providing encouraging data that GD2 is an effective and suitable target for the application of adoptive T-cell therapy in MB. Thanks to an in vitro live cell imaging system, it was possible to prove the prompt responsiveness of CAR.GD2 T-cells to recognize and kill MB tumoral cells. Indeed, monitoring cocultures between D283 Med cell line and CAR.GD2 T-cells from the very early time-point (1 hour) to the endpoint (5 days), a significant tumor control was observed already at 12 hours, thus providing encouraging data about the relevance of CAR. GD2 T-cells to eradicate MB tumors. It was also demonstrated that the tumor recognition and elimination strictly depend from the level of antigen expression. Indeed, DAUDI cells with a dismal expression of GD2 antigen, in terms of both percentages of GD2+ cells and GD2 MFI, were not fully controlled in the vitro models, with the resistant cells being characterized by a low GD2 MFI. As a closer step to mimic a human pathophysiological condition, two in vivo orthotopic mice models of MB were established. In line with the in vitro data, CAR. GD2-T cells were able to exert a significant control of tumor growth (as assessed toward bioluminescence monitoring) and, as consequence, mice bearing MB tumors and treated with CAR. GD2-T cells show a significant prolonged OS respect to mice receiving NT T-cells. Concerning the delivery route for CAR T cells, it is well known that could significantly impact the response in patients affected by solid tumors, especially for brain tumors⁷⁸. In our study, systemic i.v. delivery of CAR T cells was able to exert a significant anti-tumor response in the animal model of MB, being associated to a significant disease-free survival. a high number of tumors infiltrating T-cells. Notably, it has been reported that the use of CAR T-cells could induce fatal side-effects, such as cytokine release syndrome^{75, 79} or neurological toxicities⁸⁰. For this reason, in the construct the iC9 suicide gene was included, able to promptly eradicate the genetically modified cells, as proved in several models^{51, 72, 81}. It has been now proved that the systemic administration of the dimerizing drug AP1903 led to the significant reduction of both circulating CAR.GD2 T-cells and brain/tumor-infiltrating human CD3+ cells in mouse cerebellum. Therefore, in considering the clinical investigation of this approach in MB, the incorporation of a suicide gene may represent a relevant approach to mitigate potential risks related to CAR-T cells including autonomous proliferation and neurotoxicity. In light of these preclinical findings, a phase I/II Clinical trial of CAR. GD2 T-cell therapy to treat patients with MB GD2+ (NCT05298995) is in development, providing them a helpful and innovative therapeutic strategy.

### REFERENCES

1 Liu, J. et al. Ganglioside GD3 synthase (GD3S), a novel cancer drug target. Acta Pharm Sin B 8, 713-720, doi: 10.1016/j.apsb.2018.07.009 (2018).
2 Nazha, B., Inal, C. & Owonikoko, T. K. Disialoganglioside GD2 Expression in Solid Tumors and Role as a Target for Cancer Therapy. Front Oncol 10, 1000, doi: 10.3389/fonc.2020.01000 (2020).
3 Ravindranath, M. H., Muthugounder, S. & Presser, N. Ganglioside signatures of primary and nodal metastatic melanoma cell lines from the same patient. Melanoma Res 18, 47-55, doi: 10.1097/CMR.0b013e3282f43acf (2008).
4 Strobel, S. B. et al. Expression of Potential Targets for Cell-Based Therapies on Melanoma Cells. Life (Basel) 11, doi: 10.3390/life11040269 (2021).
5 Dobrenkov, K., Ostrovnaya, I., Gu, J., Cheung, I. Y. & Cheung, N. K. Oncotargets GD2 and GD3 are highly expressed in sarcomas of children, adolescents, and young adults. Pediatr Blood Cancer 63, 1780-1785, doi: 10.1002/pbc.26097 (2016).
6 Battula, V. L. et al. Ganglioside GD2 identifies breast cancer stem cells and promotes tumorigenesis. J Clin Invest 122, 2066-2078, doi: 10.1172/JCI59735 (2012).
7 Schulz, G. et al. Detection of ganglioside GD2 in tumor tissues and sera of neuroblastoma patients. Cancer Res 44, 5914-5920 (1984).
8 Sujitjoon, J. et al. GD2-specific chimeric antigen receptor-modified T cells targeting retinoblastoma—assessing tumor and T cell interaction. Transl Oncol 14, 100971, doi: 10.1016/j.tranon.2020.100971 (2021).
9 Cheresh, D. A., Rosenberg, J., Mujoo, K., Hirschowitz, L. & Reisfeld, R. A. Biosynthesis and expression of the disialoganglioside GD2, a relevant target antigen on small cell lung carcinoma for monoclonal antibody-mediated cytolysis. Cancer Res 46, 5112-5118 (1986).
10 Longee, D. C. et al. Disialoganglioside GD2 in human neuroectodermal tumor cell lines and gliomas. Acta Neuropathol 82, 45-54, doi: 10.1007/BF00310922 (1991).
11 Wingerter, A. et al. Exploiting Gangliosides for the Therapy of Ewing's Sarcoma and H3K27M-Mutant Diffuse Midline Glioma. Cancers (Basel) 13, doi: 10.3390/cancers13030520 (2021).
12 Jones, D. T. W. et al. Molecular characteristics and therapeutic vulnerabilities across paediatric solid tumours. Nat Rev Cancer 19, 420-438, doi: 10.1038/s41568-019-0169-x (2019).
13 Dyson, K. A. et al. Emerging trends in immunotherapy for pediatric sarcomas. J Hematol Oncol 12, 78, doi: 10.1186/s13045-019-0756-z (2019).
14 Hingorani, P. et al. Current state of pediatric sarcoma biology and opportunities for future discovery: A report from the sarcoma translational research workshop. Cancer Genet 209, 182-194, doi: 10.1016/j.cancergen.2016.03.004 (2016).
15 Thanindratarn, P., Dean, D. C., Nelson, S. D., Hornicek, F. J. & Duan, Z. Chimeric antigen receptor T (CAR-T) cell immunotherapy for sarcomas: From mechanisms to potential clinical applications. Cancer Treat Rev 82, 101934, doi: 10.1016/j.ctrv.2019.101934 (2020).
16 Ladenstein, R. et al. Primary disseminated multifocal Ewing sarcoma: results Euro-EWING 99 trial. J Clin Oncol 28, 3284-3291, of the doi: 10.1200/JCO.2009.22.9864 (2010).
17 Delattre, O. et al. Gene fusion with an ETS DNA-binding domain caused by chromosome translocation in human tumours. Nature 359, 162-165, doi: 10.1038/359162a0 (1992).
18 Knutson, S. K. et al. Selective inhibition of EZH2 by EPZ-6438 leads to potent antitumor activity in EZH2-mutant non-Hodgkin lymphoma. Mol Cancer Ther 13, 842-854, doi: 10.1158/1535-7163.MCT-13-0773 (2014).
19 Kailayangiri, S. et al. EZH2 Inhibition in Ewing Sarcoma Upregulates G (D2) Expression for Targeting with Gene-Modified T Cells. Mol Ther 27, 933-946, doi: 10.1016/j.ymthe.2019.02.014 (2019).
20 Richter, G. H. et al. EZH2 is a mediator of EWS/FLI1 driven tumor growth and metastasis blocking endothelial and neuro-ectodermal differentiation. Proc Natl Acad Sci USA 106, 5324-5329, doi: 10.1073/pnas.0810759106 (2009).
21 Wiebel, M. et al. Surface expression of the immunotherapeutic target G(D2) in osteosarcoma depends on cell confluency. Cancer Rep (Hoboken) 4, e1394, doi: 10.1002/cnr2.1394 (2021).
22 Subramanian, S. & Ahmad, T. in StatPearls (2023).
23 Quintarelli, C. et al. Innovative and Promising Strategies to Enhance Effectiveness of Immunotherapy for CNS Tumors: Where Are We? Front Immunol 12, 634031, doi: 10.3389/fimmu.2021.634031 (2021).
24 Fangusaro, J. & Bandopadhayay, P. Advances in the classification and treatment of pediatric brain tumors. Curr Opin Pediatr 33, 26-32, doi: 10.1097/MOP.0000000000000975 (2021).
25 Louis, D. N. et al. The 2016 World Health Organization Classification of Tumors of the Central Nervous System: a summary. Acta Neuropathol 131, 803-820, doi: 10.1007/s00401-016-1545-1 (2016).
26 Bandopadhayay, P. et al. Long-term outcome of 4,040 children diagnosed with pediatric low-grade gliomas: an analysis of the Surveillance Epidemiology and End Results (SEER) database. Pediatr Blood Cancer 61, 1173-1179, doi: 10.1002/pbc.24958 (2014).
27 Ater, J. L. et al. Randomized study of two chemotherapy regimens for treatment of low-grade glioma in young children: a report from the Children's Oncology Group. J Clin Oncol 30 2641-2647, doi: 10.1200/JCO.2011.36.6054 (2012).
28 Ostrom, Q. T. et al. CBTRUS Statistical Report: Primary Brain and Other Central Nervous System Tumors Diagnosed in the United States in 2012-2016. Neuro Oncol 21, v1-v100, doi: 10.1093/neuonc/noz150 (2019).
29 Jones, D. T. W., Bandopadhayay, P. & Jabado, N. The Power of Human Cancer Genetics as Revealed by Low-Grade Gliomas. Annu Rev Genet 53, 483-503, doi: 10.1146/annurev-genet-120417-031642 (2019).
30 Jones, C. et al. Pediatric high-grade glioma: biologically and clinically in need of new thinking. Neuro Oncol 19, 153-161, doi: 10.1093/neuonc/now101 (2017).
31 Mount, C. W. et al. Potent antitumor efficacy of anti-GD2 CAR T cells in H3-K27M (+) diffuse midline gliomas. Nat Med 24, 572-579, doi: 10.1038/s41591-018-0006-x (2018).
32 Cavalli, F. M. G. et al. Heterogeneity within the PF-EPN-B ependymoma subgroup. Acta Neuropathol 136, 227-237, doi: 10.1007/s00401-018-1888-x (2018).
33 Maier, H., Dalianis, T. & Kostopoulou, O. N. New Approaches in Targeted Therapy for Medulloblastoma in Children. Anticancer Res 41, 1715-1726, doi: 10.21873/anticanres. 14936 (2021).
34 Northcott, P. A. et al. Medulloblastoma. Nat Rev Dis Primers 5, 11, doi: 10.1038/s41572-019-0063-6 (2019).
35 Ribi, K. et al. Outcome of medulloblastoma in children: long-term complications and quality of life. Neuropediatrics 36, 357-365, doi: 10.1055/s-2005-872880 (2005).
36 Jakacki, R. I. et al. Outcome of children with metastatic medulloblastoma treated with carboplatin during craniospinal radiotherapy: a Children's Oncology Group Phase I/II study. J Clin Oncol 30, 2648-2653, doi: 10.1200/JCO.2011.40.2792 (2012).
37 Martin, A. M., Raabe, E., Eberhart, C. & Cohen, K. J. Management of pediatric and adult patients with medulloblastoma. Curr Treat Options Oncol 15, 581-594, doi: 10.1007/s11864-014-0306-4 (2014).
38 Gajjar, A. et al. Risk-adapted craniospinal radiotherapy followed by high-dose chemotherapy and stem-cell rescue in children with newly diagnosed medulloblastoma (St Jude Medulloblastoma-96): long-term results from a prospective, multicentre trial. Lancet Oncol 7, 813-820, doi: 10.1016/S1470-2045 (06) 70867-1 (2006).
39 Cooney, T., Lindsay, H., Leary, S. & Wechsler-Reya, R. Current studies and future directions for medulloblastoma: A review from the pacific pediatric neuro-oncology consortium (PNOC) disease working group. Neoplasia 35, 100861, doi: 10.1016/j.neo.2022.100861 (2023).
40 Ellison, D. W. et al. Medulloblastoma: clinicopathological correlates of SHH, WNT, and non-SHH/WNT molecular subgroups. Acta Neuropathol 121, 381-396, doi: 10.1007/s00401-011-0800-8 (2011).
41 Clifford, S. C. et al. Wnt/Wingless pathway activation and chromosome 6 loss characterize a distinct molecular sub-group of medulloblastomas associated with a favorable prognosis. Cell Cycle 5, 2666-2670, doi: 10.4161/cc.5.22.3446 (2006).
42 Quinlan, A. & Rizzolo, D. Understanding medulloblastoma. JAAPA 30, 30-36, doi: 10.1097/01.JAA.0000524717.71084.50 (2017).
43 Mennel, H. D., Bosslet, K., Geissel, H. & Bauer, B. L. Immunohistochemically visualized localisation of gangliosides Glac2 (GD3) and Gtri2 (GD2) in cells of human intracranial tumors. Exp Toxicol Pathol 52, 277-285, doi: 10.1016/S0940-2993 (00) 80046-9 (2000).
44 Packer, R. J. et al. Atypical teratoid/rhabdoid tumor of the central nervous system: report on workshop. J Pediatr Hematol Oncol 24, 337-342, doi: 10.1097/00043426-200206000-00004 (2002).
45 Dardis, C. et al. Atypical Teratoid Rhabdoid Tumor: Two Case Reports and an Analysis of Adult Cases with Implications for Pathophysiology and Treatment. Front Neurol 8, 247, doi: 10.3389/fneur.2017.00247 (2017).
46 Khan, S. et al. Clinical phenotypes and prognostic features of embryonal tumours with multi-layered rosettes: a Rare Brain Tumor Registry study. Lancet Child Adolesc Health 5, 800-813, doi: 10.1016/S2352-4642 (21) 00245-5 (2021).
47 Ladenstein, R. et al. Interleukin 2 with anti-GD2 antibody ch14.18/CHO (dinutuximab beta) in patients with high-risk neuroblastoma (HR-NBL1/SIOPEN): a multicentre, randomised, phase 3 trial. Lancet Oncol 19, 1617-1629, doi: 10.1016/S1470-2045 (18) 30578-3 (2018).
48 Kushner, B. H. et al. Humanized 3F8 Anti-GD2 Monoclonal Antibody Dosing With Granulocyte-Macrophage Colony-Stimulating Factor in Patients With Resistant Neuroblastoma: A Phase 1 Clinical Trial. JAMA Oncol 4, 1729-1735, doi: 10.1001/jamaoncol.2018.4005 (2018).
49 Kramer, K. et al. A phase II study of radioimmunotherapy with intraventricular (131) 1-3F8 for medulloblastoma. Pediatr Blood Cancer 65, doi: 10.1002/pbc.26754 (2018).
50 Kramer, K. et al. Compartmental intrathecal radioimmunotherapy: results for treatment for metastatic CNS neuroblastoma. J Neurooncol 97, 409-418, doi: 10.1007/s11060-009-0038-7 (2010).
51 Quintarelli, C. et al. Choice of costimulatory domains and of cytokines determines CAR T-cell activity in neuroblastoma. Oncoimmunology 7, e1433518, doi: 10.1080/2162402X.2018.1433518 (2018)
52 Majzner, R. G. et al. GD2-CAR T cell therapy for H3K27M-mutated diffuse midline gliomas. Nature 603, 934-941, doi: 10.1038/s41586-022-04489-4 (2022).
53 Reppel, L. et al. Targeting disialoganglioside GD2 with chimeric antigen receptor-redirected T cells in lung cancer. J Immunother Cancer 10, doi: 10.1136/jitc-2021-003897 (2022).
54 Esaki, N. et al. ASC amino acid transporter 2, defined by enzyme-mediated activation of radical sources, enhances malignancy of GD2-positive small-cell lung cancer. Cancer Sci 109, 141-153, doi: 10.1111/cas. 13448 (2018).
55 Mansoori, M. et al. High GD2 expression defines breast cancer cells with enhanced invasiveness. Exp Mol Pathol 109, 25-35, doi: 10.1016/j.yexmp.2019.05.001 (2019).
56 Louis, C. U. et al. Antitumor activity and long-term fate of chimeric antigen receptor-positive T cells in patients with neuroblastoma. Blood 118, 6050-6056, doi: 10.1182/blood-2011-05-354449 (2011).
57 Pule, M. A. et al. Virus-specific T cells engineered to coexpress tumor-specific receptors: persistence and antitumor activity in individuals with neuroblastoma. Nat Med 14, 1264-1270, doi: 10.1038/nm. 1882 (2008).
58 Heczey, A. et al. CAR T Cells Administered in Combination with Lymphodepletion and PD-1 Inhibition to Patients with Neuroblastoma. Mol Ther 25, 2214-2224, doi: 10.1016/j.ymthe.2017.05.012 (2017).
59 Gargett, T. et al. Safety and biological outcomes following a phase 1 trial of GD2-specific CAR-T cells in patients with GD2-positive metastatic melanoma and other solid cancers. J Immunother Cancer 12, doi: 10.1136/jitc-2023-008659 (2024).
60 Shum, T. et al. Constitutive Signaling from an Engineered IL7 Receptor Promotes Durable Tumor Elimination by Tumor-Redirected T Cells. Cancer Discov 7, 1238-1247, doi: 10.1158/2159-8290.CD-17-0538 (2017).
61 Caforio, M. et al. GD2 redirected CAR T and activated NK-cell-mediated secretion of IFNgamma overcomes MYCN-dependent IDO1 inhibition, contributing to neuroblastoma cell immune escape. J Immunother Cancer 9, doi: 10.1136/jitc-2020-001502 (2021).
62 Tumino, N. et al. Polymorphonuclear myeloid-derived suppressor cells impair the anti-tumor efficacy of GD2.CAR T-cells in patients with neuroblastoma. J Hematol Oncol 14, 191, doi: 10.1186/s13045-021-01193-0 (2021).
63 de Billy, E. et al. Dual IGF1R/IR inhibitors in combination with GD2-CAR T-cells display a potent anti-tumor activity in diffuse midline glioma H3K27M-mutant. Neuro Oncol 24, 1150-1163, doi: 10.1093/neuonc/noab300 (2022).
64 Del Bufalo, F. et al. GD2-CART01 for Relapsed or Refractory High-Risk Neuroblastoma. N Engl J Med 388, 1284-1295, doi: 10.1056/NEJMoa2210859 (2023).
65 Gargett, T. & Brown, M. P. The inducible caspase-9 suicide gene system as a “safety switch” to limit on-target, off-tumor toxicities of chimeric antigen receptor T cells. Front Pharmacol 5, 235, doi: 10.3389/fphar.2014.00235 (2014).
66 Zhong, X. S., Matsushita, M., Plotkin, J., Riviere, I. & Sadelain, M. Chimeric antigen receptors combining 4-1BB and CD28 signaling domains augment PI3kinase/AKT/Bcl-XL activation and CD8+ T cell-mediated tumor eradication. Mol Ther 18, 413-420, doi: 10.1038/mt.2009.210 (2010).
67 Long, A. H. et al. Reduction of MDSCs with All-trans Retinoic Acid Improves CAR Therapy Efficacy for Sarcomas. Cancer Immunol Res 4, 869-880, doi: 10.1158/2326-6066.CIR-15-0230 (2016).
68 Vaswani, R. G. et al. Identification of (R)-N-((4-Methoxy-6-methyl-2-oxo-1,2-dihydropyridin-3-yl)methyl)-2-methyl-1-(1-(1-(2,2,2-trifluoroethyl) piperidin-4-yl)ethyl)-1H-indole-3-carboxamide (CPI-1205), a Potent and Selective Inhibitor of Histone Methyltransferase EZH2, Suitable for Phase I Clinical Trials for B-Cell Lymphomas. J Med Chem 59, 9928-9941, doi: 10.1021/acs.jmedchem.6b01315 (2016).
69 Duan, R., Du, W. & Guo, W. EZH2: a novel target for cancer treatment. J Hematol Oncol 13, 104, doi: 10.1186/s13045-020-00937-8 (2020).
70 Kung, P. P. et al. Optimization of Orally Bioavailable Enhancer of Zeste Homolog 2 (EZH2) Inhibitors Using Ligand and Property-Based Design Strategies: Identification of Development Candidate (R)-5,8-Dichloro-7-(methoxy (oxetan-3-yl)methyl)-2-((4-methoxy-6-methyl-2-oxo-1,2-dihydropyridin-3-yl)methyl)-3,4-dihydroisoquinolin-1 (2H)-one (PF-06821497). J Med Chem 61, 650-665, doi: 10.1021/acs.jmedchem.7b01375 (2018).
71 McCabe, M. T. et al. EZH2 inhibition as a therapeutic strategy for lymphoma mutations. with EZH2-activating Nature 492, 108-112, doi: 10.1038/nature11606 (2012).
72 Guercio, M. et al. CD28.OX40 co-stimulatory combination is associated with long in vivo persistence and high activity of CAR.CD30 T-cells.
73 Haematologica 106, 987-999, doi: 10.3324/haematol.2019.231183 (2021). Di Stasi, A., De Angelis, B. & Savoldo, B. Gene therapy to improve migration of T cells to the tumor site. Methods Mol Biol 651, 103-118, doi: 10.1007/978-1-60761-786-0_7 (2010).
74 Orlando, D. et al. Adoptive Immunotherapy Using PRAME-Specific T Cells in Medulloblastoma. Cancer Res 78, 3337-3349, doi: 10.1158/0008-5472.CAN-17-3140 (2018).
75 Brudno, J. N. & Kochenderfer, J. N. Recent advances in CAR T-cell toxicity: Mechanisms, manifestations and management. Blood Rev 34, 45-55, doi: 10.1016/j.blre.2018.11.002 (2019).
76 Erber, R. et al. Variable Expression of the Disialoganglioside GD2 in Breast Cancer Molecular Subtypes. Cancers (Basel) 13, doi: 10.3390/cancers13215577 (2021).
77 Gargett, T. et al. GD2-targeting CAR-T cells enhanced by transgenic IL-15 expression are an effective and clinically feasible therapy for glioblastoma. J Immunother Cancer 10, doi: 10.1136/jitc-2022-005187 (2022).
78 Prapa, M. et al. GD2 CAR T cells against human glioblastoma. NPJ Precis Oncol 5, 93, doi: 10.1038/s41698-021-00233-9 (2021).
79 Frey, N. & Porter, D. Cytokine Release Syndrome with Chimeric Antigen Receptor T Cell Therapy. Biol Blood Marrow Transplant 25, e123-e127, doi: 10.1016/j.bbmt.2018.12.756 (2019).
80 Gust, J. et al. Endothelial Activation and Blood-Brain Barrier Disruption in Neurotoxicity after Adoptive Immunotherapy with CD19 CAR-T Cells. Cancer Discov 7, 1404-1419, doi: 10.1158/2159-8290.CD-17-0698 (2017).
81 Guercio, M. et al. Inclusion of the Inducible Caspase 9 Suicide Gene in CAR Construct Increases Safety of CAR.CD19 T Cell Therapy in B-Cell Malignancies. Front Immunol 12, 755639, doi: 10.3389/fimmu.2021.755639 (2021).

1-34. (canceled)

35. Anti-GD2 chimeric antigen receptor comprising, from the N-terminus to the C-terminus:
a) a signal peptide,
b) an anti GD2 single chain antibody domain,
c) a hinge,
d) a trans membrane domain,
e) at least two co-stimulatory signaling domains, and
f) CD3Zeta chain sequence,
wherein said anti GD2 single chain antibody domain comprises anti GD2 VL sequence and anti GD2 VH sequence linked each other by a linker, said linker being a short flexible glycines-rich linker with a length from 7 to 14 amino acids.

36. Anti-GD2 chimeric antigen receptor according to claim 35, wherein
anti GD2 VL sequence comprises CDR1 sequence QSLVHRNGNTY (SEQ ID NO: 5), CDR2 sequence: KVS and CDR3 sequence SQSTHVP (SEQ ID NO: 7);
wherein
anti GD2 VH sequence comprises CDR1 sequence: GSSFTGYN (SEQ ID NO: 8), CDR2 sequence: IDPYYGGT (SEQ ID NO: 9) and CDR3 sequence: VSGMEY (SEQ ID NO: 10).

37. Anti GD2 chimeric antigen receptor according to claim 35, wherein
anti GD2 VL sequence comprises:
(SEQ ID NO: 11)
DILLTQTPLSLPVSLGDQASISCRSSQSLVHRNGNTYLHWYLQKPG
QSPKLLIHKVSNRFSGVPDRFSGSGSGTDFTLKISRVEAEDLGVYF
CSQSTHVPPLTFGAGTKLELKRADAAPTVSIFP,
and
anti GD2 VH sequence comprises:
(SEQ ID NO: 12)
EVKLQQSGPSLVEPGASVMISCKASGSSFTGYNMNWVRQNIGKSLE
WIGAIDPYYGGTSYNQKFKGRATLTVDKSSSTAYMHLKSLTSEDSA
VYYCVSGMEYWGQGTSVTVSS.

38. Anti-GD2 chimeric antigen receptor according to claim 35, wherein said hinge comprises one or more of the following hinges:
IgG-based hinge of sequence
(SEQ ID NO: 23)
AKTTPPSVYGRVTVSSAEPKSCDKTHTCPPCP;
hinge Spacer-CD8α of sequence
(SEQ ID NO: 24)
PAPRPPTPAPTIASQPLSLRPEACRPAAGGAVHTRGLDFA;
CD8stalk of sequence
(SEQ ID NO: 25)
TTTPAPRPPTPAPTIASQPLSLRPEACRPAAGGAVHTRGLDFACD;
hinge CD28 of sequence
(SEQ ID NO: 26)
EVMYPPPYLDNEKSNGTIIHVKGKHLCPSPLFPGPSKP;
hinge CH2-CH3 of sequence
(SEQ ID NO: 27)
ESKYGPPCPSCPAPEFLGGPSVFLFPPKPKDTLMISRTPEVTCVVV
DVSQEDPEVQFNWYVDGVEVHNAKTKPREEQFNSTYRVVSVLTVLH
QDWLNGKEYKCKVSNKGLPSSIEKTISKAKGQPREPQVYTLPPSQE
EMTKNQVSLTCLVKGFYPSDIAVEWESNGQPENNYKTTPPVLDSDG
SFFLYSRLTVDKSRWQEGNVFSCSVMHEALHNHYTQKSLSLSLGK;
and
hinge CH3 of sequence
(SEQ ID NO: 28)
ESKYGPPCPSCPGQPREPQVYTLPPSQEEMTKNQVSLTCLVKGFYP
SDIAVEWESNGQPENNYKTTPPVLDSDGSFFLYSRLTVDKSRWQEG
NVFSCSVMHEALHNHYTQKSLSLSLGK;

39. Anti-GD2 chimeric antigen receptor according to claim 35, wherein said trans membrane domain is chosen from the group consisting of CD8aTM: CDIYIWAPLAGTCGVLLLSLVIT (SEQ ID NO: 6); and CD28TM: FWVLVVVGGVLACYSLLVTVAFIIFWV (SEQ ID NO: 29).

40. Anti-GD2 chimeric antigen receptor according to claim 35, wherein said at least two co-stimulatory signaling domains are chosen from the group consisting of:
A) a sequence obtained by linking:
CD28 cytoplasmic sequence:
(SEQ ID NO: 30)
RSKRSRLLHSDYMNMTPRRPGPTRKHYQPYAPPRDFAAYRS
to
OX40 sequence:
(SEQ ID NO: 32)
RDQRLPPDAHKPPGGGSFRTPIQEEQADAHSTLAKI,
wherein CD28 cytoplasmic sequence is positioned before or after OX40 sequence;
B) a sequence obtained by linking:
CD28 cytoplasmic sequence:
RSKRSRLLHSDYMNMTPRRPGPTRKHYQPYAPPRDFAAYRS (SEQ ID NO: 30) to
CD137 (4-1BB) sequence:
KRGRKKLLYIFKQPFMRPVQTTQEEDGCSCRFPEEEEGGCEL (SEQ ID NO: 31), wherein CD28 cytoplasmic sequence is positioned before or after CD137 (4-1BB) sequence;
C) a sequence obtained by linking:
OX40 sequence:
RDQRLPPDAHKPPGGGSFRTPIQEEQADAHSTLAKI (SEQ ID NO: 32) to CD28 cytoplasmic sequence:
RSKRSRLLHSDYMNMTPRRPGPTRKHYQPYAPPRDFAAYRS (SEQ ID NO: 30), wherein OX40 sequence is positioned before or after CD28 cytoplasmic sequence; and
D) a sequence obtained by linking:
OX40 sequence:
RDQRLPPDAHKPPGGGSFRTPIQEEQADAHSTLAKI (SEQ ID NO: 32) to CD137 (4-1BB) sequence:
KRGRKKLLYIFKQPFMRPVQTTQEEDGCSCRFPEEEEGGCEL (SEQ ID NO: 31), wherein OX40 sequence is positioned before or after CD137 (4-1BB) sequence.

41. Anti-GD2 chimeric antigen receptor according to claim 35, wherein CD3-Zeta chain sequence is RVKFSRSADAPAYQQGQNQLYNELNLGRREEYDVLDKRRGRDPEMGGKPRRKNP QEGLYNELQKDKMAEAYSEIGMKGERRRGKGHDGLYQGLSTATKDTYDALHMQA LPPR* (SEQ ID NO: 53).

42. Anti-GD2 chimeric antigen receptor according to claim 35, further comprising cytoplasmic moiety of CD8cyt of sequence LYCNHRN (SEQ ID NO: 51) between the trans membrane domain and the co-stimulatory signaling domain.

43. Anti-GD2 chimeric antigen receptor according to claim 35, wherein said signal peptide comprises er consists of MEFGLSWLFLVAILKGVQCSR (SEQ ID NO: 3).

44. Anti-GD2 chimeric antigen receptor according to claim 35, wherein said anti-GD2 chimeric antigen receptor comprises or consists of the following sequence:
(SEQ ID NO: 39)
MEFGLSWLFLVAILKGVQCSRDILLTQTPLSLPVSLGDQASISCR
SSQSLVHRNGNTYLHWYLQKPGQSPKLLIHKVSNRFSGVPDRFSG
SGSGTDFTLKISRVEAEDLGVYFCSQSTHVPPLTFGAGTKLELKR
ADAAPTVSIFPGSGGGGSGGEVKLQQSGPSLVEPGASVMISCKAS
GSSFTGYNMNWVRQNIGKSLEWIGAIDPYYGGTSYNQKFKGRATL
TVDKSSSTAYMHLKSLTSEDSAVYYCVSGMEYWGQGTSVTVSSAK
TTPPSVYGRVTVSSAEPKSCDKTHTCPPCPDPKFWVLVVVGGVLA
CYSLLVTVAFIIFWVRSKRSRLLHSDYMNMTPRRPGPTRKHYQPY
APPRDFAAYRSKRGRKKLLYIFKQPFMRPVQTTQEEDGCSCRFPE
EEEGGCELRVKFSRSADAPAYQQGQNQLYNELNLGRREEYDVLDK
RRGRDPEMGGKPRRKNPQEGLYNELQKDKMAEAYSEIGMKGERRR
GKGHDGLYQGLSTATKDTYDALHMQALPPR*.

45. Nucleotide sequence or vector comprising the nucleotide sequence, said nucleotide sequence comprising a nucleotide sequence that encodes an anti-GD2 chimeric antigen receptor as defined in claim 35.

46. Nucleotide sequence or vector according to claim 45, wherein
anti GD2 VL sequence is encoded by the
nucleotide sequence
(SEQ ID NO: 36)
GATATTTTGCTGACCCAAACTCCACTCTCCCTGCCTGTCAGTCTT
GGAGATCAAGCCTCCATCTCTTGCAGATCTAGTCAGAGTCTTGTA
CACCGTAATGGAAACACCTATTTACATTGGTACCTGCAGAAGCCA
GGCCAGTCTCCAAAGCTCCTGATTCACAAAGTTTCCAACCGATTT
TCTGGGGTCCCAGACAGGTTCAGTGGCAGTGGATCAGGGACAGAT
TTCACACTCAAGATCAGCAGAGTGGAGGCTGAGGATCTGGGAGTT
TATTTCTGTTCTCAAAGTACACATGTTCCTCCGCTCACGTTCGGT
GCTGGGACCAAGCTGGAGCTGAAACGGGCTGATGCTGCACCAACT
GTATCCATCTTCCCA,
and
anti GD2 VH sequence is encoded by the
nucleotide sequence
(SEQ ID NO: 37)
GAGGTGAAGCTTCAGCAGTCTGGACCTAGCCTGGTGGAGCCTGGC
GCTTCAGTGATGATATCCTGCAAGGCTTCTGGTTCCTCATTCACT
GGCTACAACATGAACTGGGTGAGGCAGAACATTGGAAAGAGCCTT
GAATGGATTGGAGCTATTGATCCTTACTATGGTGGAACTAGCTAC
AACCAGAAGTTCAAGGGCAGGGCCACATTGACTGTAGACAAATCG
TCCAGCACAGCCTACATGCACCTCAAGAGCCTGACATCTGAGGAC
TCTGCAGTCTATTACTGTGTAAGCGGAATGGAGTACTGGGGTCAA
GGAACCTCAGTCACCGTCTCCTCA.

47. Nucleotide sequence or vector according to claim 45, wherein the nucleotide sequence encoding anti-GD2 chimeric antigen receptor is:
(SEQ ID NO: 52)
ATGGAGTTTGGGCTGAGCTGGCTTTTTCTTGTGGCTATTTTAAAA
GGTGTCCAGTGCTCTAGAGATATTTTGCTGACCCAAACTCCACTC
TCCCTGCCTGTCAGTCTTGGAGATCAAGCCTCCATCTCTTGCAGA
TCTAGTCAGAGTCTTGTACACCGTAATGGAAACACCTATTTACAT
TGGTACCTGCAGAAGCCAGGCCAGTCTCCAAAGCTCCTGATTCAC
AAAGTTTCCAACCGATTTTCTGGGGTCCCAGACAGGTTCAGTGGC
AGTGGATCAGGGACAGATTTCACACTCAAGATCAGCAGAGTGGAG
GCTGAGGATCTGGGAGTTTATTTCTGTTCTCAAAGTACACATGTT
CCTCCGCTCACGTTCGGTGCTGGGACCAAGCTGGAGCTGAAACGG
GCTGATGCTGCACCAACTGTATCCATCTTCCCAGGCTCGGGCGGT
GGTGGGTCGGGTGGCGAGGTGAAGCTTCAGCAGTCTGGACCTAGC
CTGGTGGAGCCTGGCGCTTCAGTGATGATATCCTGCAAGGCTTCT
GGTTCCTCATTCACTGGCTACAACATGAACTGGGTGAGGCAGAAC
ATTGGAAAGAGCCTTGAATGGATTGGAGCTATTGATCCTTACTAT
GGTGGAACTAGCTACAACCAGAAGTTCAAGGGCAGGGCCACATTG
ACTGTAGACAAATCGTCCAGCACAGCCTACATGCACCTCAAGAGC
CTGACATCTGAGGACTCTGCAGTCTATTACTGTGTAAGCGGAATG
GAGTACTGGGGTCAAGGAACCTCAGTCACCGTCTCCTCAGCCAAA
ACGACACCCCCATCAGTCTATGGAAGGGTCACCGTCTCTTCAGCG
GAGCCCAAATCTTGTGACAAAACTCACACATGCCCACCGTGCCCG
GATCCCAAATTTTGGGTGCTGGTGGTGGTTGGTGGAGTCCTGGCT
TGCTATAGCTTGCTAGTAACAGTGGCCTTTATTATTTTCTGGGTG
AGGAGTAAGAGGAGCAGGCTCCTGCACAGTGACTACATGAACATG
ACTCCCCGCCGCCCCGGGCCCACCCGCAAGCATTACCAGCCCTAT
GCCCCACCACGCGACTTCGCAGCCTATCGCTCCAAACGGGGCAGA
AAGAAACTCCTGTATATATTCAAACAACCATTTATGAGACCAGTA
CAAACTACTCAAGAGGAAGATGGCTGTAGCTGCCGATTTCCAGAA
GAAGAAGAAGGAGGATGTGAACTGAGAGTGAAGTTCAGCAGGAGC
GCAGACGCCCCCGCGTACCAGCAGGGCCAGAACCAGCTCTATAAC
GAGCTCAATCTAGGACGAAGAGAGGAGTACGATGTTTTGGACAAG
AGACGTGGCCGGGACCCTGAGATGGGGGGAAAGCCGAGAAGGAAG
AACCCTCAGGAAGGCCTGTACAATGAACTGCAGAAAGATAAGATG
GCGGAGGCCTACAGTGAGATTGGGATGAAAGGCGAGCGCCGGAGG
GGCAAGGGGCACGATGGCCTTTACCAGGGTCTCAGTACAGCCACC
AAGGACACCTACGACGCCCTTCACATGCAGGCCCTGCCCCCTCGC
TAA.

48. Nucleotide or vector sequence according to claim 45, said nucleotide sequence further comprising a nucleotide sequence encoding a suicide gene inducible amino acid sequence linked to the nucleotide sequence encoding said chimeric antigen receptor by a nucleotide sequence encoding a 2A self-cleaving peptide.

49. Nucleotide sequence or vector according to claim 48, wherein the suicide gene inducible amino acid sequence comprises a chimeric Caspase-9 polypeptide, a herpes simplex virus thymidine kinase, or ACD19 sequence.

50. Nucleotide sequence or vector according to claim 45, wherein the nucleotide sequence is:
(SEQ ID NO: 50)
ATGCTCGAGGGAGTGCAGGTGGAGACTATCTCCCCAGGAGACGGG
CGCACCTTCCCCAAGCGCGGCCAGACCTGCGTGGTGCACTACACC
GGGATGCTTGAAGATGGAAAGAAAGTTGATTCCTCCCGGGACAGA
AACAAGCCCTTTAAGTTTATGCTAGGCAAGCAGGAGGTGATCCGA
GGCTGGGAAGAAGGGGTTGCCCAGATGAGTGTGGGTCAGAGAGCC
AAACTGACTATATCTCCAGATTATGCCTATGGTGCCACTGGGCAC
CCAGGCATCATCCCACCACATGCCACTCTCGTCTTCGATGTGGAG
CTTCTAAAACTGGAATCTGGCGGTGGATCCGGAGTCGACGGATTT
GGTGATGTCGGTGCTCTTGAGAGTTTGAGGGGAAATGCAGATTTG
GCTTACATCCTGAGCATGGAGCCCTGTGGCCACTGCCTCATTATC
AACAATGTGAACTTCTGCCGTGAGTCCGGGCTCCGCACCCGCACT
GGCTCCAACATCGACTGTGAGAAGTTGCGGCGTCGCTTCTCCTCG
CTGCATTTCATGGTGGAGGTGAAGGGCGACCTGACTGCCAAGAAA
ATGGTGCTGGCTTTGCTGGAGCTGGCGCgGCAGGACCACGGTGCT
CTGGACTGCTGCGTGGTGGTCATTCTCTCTCACGGCTGTCAGGCC
AGCCACCTGCAGTTCCCAGGGGCTGTCTACGGCACAGATGGATGC
CCTGTGTCGGTCGAGAAGATTGTGAACATCTTCAATGGGACCAGC
TGCCCCAGCCTGGGAGGGAAGCCCAAGCTCTTTTTCATCCAGGCC
TGTGGTGGGGAGCAGAAAGACCATGGGTTTGAGGTGGCCTCCACT
TCCCCTGAAGACGAGTCCCCTGGCAGTAACCCCGAGCCAGATGCC
ACCCCGTTCCAGGAAGGTTTGAGGACCTTCGACCAGCTGGACGCC
ATATCTAGTTTGCCCACACCCAGTGACATCTTTGTGTCCTACTCT
ACTTTCCCAGGTTTTGTTTCCTGGAGGGACCCCAAGAGTGGCTCC
TGGTACGTTGAGACCCTGGACGACATCTTTGAGCAGTGGGCTCAC
TCTGAAGACCTGCAGTCCCTCCTGCTTAGGGTCGCTAATGCTGTT
TCGGTGAAAGGGATTTATAAACAGATGCCTGGTTGCTTTAATTTC
CTCCGGAAAAAACTTTTCTTTAAAACATCAGCTAGCAGAGCCGAG
GGCAGGGGAAGTCTTCTAACATGCGGGGACGTGGAGGAAAATCCC
GGGCCCATGGAGTTTGGGCTGAGCTGGCTTTTTCTTGTGGCTATT
TTAAAAGGTGTCCAGTGCTCTAGAGATATTTTGCTGACCCAAACT
CCACTCTCCCTGCCTGTCAGTCTTGGAGATCAAGCCTCCATCTCT
TGCAGATCTAGTCAGAGTCTTGTACACCGTAATGGAAACACCTAT
TTACATTGGTACCTGCAGAAGCCAGGCCAGTCTCCAAAGCTCCTG
ATTCACAAAGTTTCCAACCGATTTTCTGGGGTCCCAGACAGGTTC
AGTGGCAGTGGATCAGGGACAGATTTCACACTCAAGATCAGCAGA
GTGGAGGCTGAGGATCTGGGAGTTTATTTCTGTTCTCAAAGTACA
CATGTTCCTCCGCTCACGTTCGGTGCTGGGACCAAGCTGGAGCTG
AAACGGGCTGATGCTGCACCAACTGTATCCATCTTCCCAGGCTCG
GGCGGTGGTGGGTCGGGTGGCGAGGTGAAGCTTCAGCAGTCTGGA
CCTAGCCTGGTGGAGCCTGGCGCTTCAGTGATGATATCCTGCAAG
GCTTCTGGTTCCTCATTCACTGGCTACAACATGAACTGGGTGAGG
CAGAACATTGGAAAGAGCCTTGAATGGATTGGAGCTATTGATCCT
TACTATGGTGGAACTAGCTACAACCAGAAGTTCAAGGGCAGGGCC
ACATTGACTGTAGACAAATCGTCCAGCACAGCCTACATGCACCTC
AAGAGCCTGACATCTGAGGACTCTGCAGTCTATTACTGTGTAAGC
GGAATGGAGTACTGGGGTCAAGGAACCTCAGTCACCGTCTCCTCA
GCCAAAACGACACCCCCATCAGTCTATGGAAGGGTCACCGTCTCT
TCAGCGGAGCCCAAATCTTGTGACAAAACTCACACATGCCCACCG
TGCCCGGATCCCAAATTTTGGGTGCTGGTGGTGGTTGGTGGAGTC
CTGGCTTGCTATAGCTTGCTAGTAACAGTGGCCTTTATTATTTTC
TGGGTGAGGAGTAAGAGGAGCAGGCTCCTGCACAGTGACTACATG
AACATGACTCCCCGCCGCCCCGGGCCCACCCGCAAGCATTACCAG
CCCTATGCCCCACCACGCGACTTCGCAGCCTATCGCTCCAAACGG
GGCAGAAAGAAACTCCTGTATATATTCAAACAACCATTTATGAGA
CCAGTACAAACTACTCAAGAGGAAGATGGCTGTAGCTGCCGATTT
CCAGAAGAAGAAGAAGGAGGATGTGAACTGAGAGTGAAGTTCAGC
AGGAGCGCAGACGCCCCCGCGTACCAGCAGGGCCAGAACCAGCTC
TATAACGAGCTCAATCTAGGACGAAGAGAGGAGTACGATGTTTTG
GACAAGAGACGTGGCCGGGACCCTGAGATGGGGGGAAAGCCGAGA
AGGAAGAACCCTCAGGAAGGCCTGTACAATGAACTGCAGAAAGAT
AAGATGGCGGAGGCCTACAGTGAGATTGGGATGAAAGGCGAGCGC
CGGAGGGGCAAGGGGCACGATGGCCTTTACCAGGGTCTCAGTACA
GCCACCAAGGACACCTACGACGCCCTTCACATGCAGGCCCTGCCC
CCTCGCTAA.

51. Cell comprising the anti-GD2 chimeric antigen receptor as defined in claim 35 and/or the vector as defined in claim 45.

52. Cell according to claim 51, wherein said cell further comprises a suicide gene inducible amino acid sequence.

53. Cell according to claim 52, wherein the chimeric Caspase-9 polypeptide comprises:
iCasp9 cassette:
(SEQ ID NO: 1)
MLEGVQVETISPGDGRTFPKRGQTCVVHYTGMLEDGKKVDSSRDR
NKPFKFMLGKQEVIRGWEEGVAQMSVGQRAKLTISPDYAYGATGH
PGIIPPHATLVFDVELLKLESGGGSGVDGFGDVGALESLRGNADL
AYILSMEPCGHCLIINNVNFCRESGLRTRTGSNIDCEKLRRRFSS
LHFMVEVKGDLTAKKMVLALLELARQDHGALDCCVVVILSHGCQA
SHLQFPGAVYGTDGCPVSVEKIVNIFNGTSCPSLGGKPKLFFIQA
CGGEQKDHGFEVASTSPEDESPGSNPEPDATPFQEGLRTFDQLDA
ISSLPTPSDIFVSYSTFPGFVSWRDPKSGSWYVETLDDIFEQWAH
SEDLQSLLLRVANAVSVKGIYKQMPGCFNFLRKKLFFKTSAS,
which is linked by a linker, to:
a T2A peptide (2A):
(SEQ ID NO: 2)
EGRGSLLTCGDVEENPGP.

54. Pharmaceutical composition comprising the nucleotide sequence or vector according to claim 45, or the cell according to claim 51, together with one or more excipients and/or adjuvants.

55. Kit of parts comprising:
A) an anti-GD2 chimeric antigen receptor as defined in claim 35, or a nucleotide sequence or a vector according to claim 45, or a cell according to claim 51, or a pharmaceutical composition according to claim 54; and
B) an enhancer of Zeste Homolog 1 or 2 (EZH2) inhibitor or a pharmaceutical composition comprising an enhancer of Zeste Homolog 1 or 2 (EZH2) inhibitor and one or more excipients and/or adjuvants,
wherein A) and B) are to administered separately or sequentially.

56. Kit according to claim 55, wherein said enhancer of Zeste Homolog 2 inhibitor is chosen from the group consisting of Tazemetostat, CPI-1205, PF-06821497, and GSK126.

57. A method of treating a patient, the method comprising administering to the patient: Anti-GD2 chimeric antigen receptor according to claim 35, nucleotide sequence or vector according to claim 45, cell according to claim 51, or pharmaceutical composition according to claim 54, or kit according to claim 55.

58. A method of treating a neuroblastoma in a patient, the method comprising administering to the patient: Anti-GD2 chimeric antigen receptor according to claim 35, nucleotide sequence or vector according to claim 45, cell according to claim 51, pharmaceutical composition according to claim 54, or kit according to claim 55.

59. A method of treating a solid tumor in a patient, wherein the solid tumor is chosen from the group consisting of extra-cranic tumors; sarcomas; brain tumors; adult-type diffuse gliomas; pediatric-type diffuse low-grade gliomas; pediatric-type diffuse high-grade gliomas; astrocytic gliomas; medulloblastoma; glioneuronal and neuronal tumors; ependymal tumors; choroid plexus tumors; embryonal tumors; pineal tumors; cranial and paraspinal nerve tumors; retinoblastoma; melanoma; lung cancer; gastric cancer; bladder cancer, head and neck cancer; ovarian cancer; breast cancer; pancreatic cancer; and colorectal cancer; the method comprising administering to the patient Anti-GD2 chimeric antigen receptor according to claim 35; nucleotide sequence or vector according to claim 45; cell according to claim 51; pharmaceutical composition according to claim 54; or kit according to claim 55.

60. The method of claim 59, wherein the sarcoma is chosen from osteosarcoma and rhabdomyosarcoma; and the medulloblastoma is chosen from SHH, G3, G4 and WNT medulloblastoma subgroups.

61. The method of claim 59, wherein the solid tumor is a tumor having an expression of GD2 higher than the expression of GD2 in healthy peripheral blood mononuclear cells, wherein the expression of GD2 is measured as the percentage of GD2+ cells with respect to the total number of cells of the tumor or of the healthy peripheral blood mononuclear cells.


SEQUENCE LISTING (53 sequences)

SEQ ID NO: 1 - protein, 402 aa, synthetic construct
MLEGVQVETISPGDGRTFPKRGQTCVVHYTGMLEDGKKVDSSRDRNKPFKFMLGKQEVIRGWEEGVAQMSVGQRAKLTISPDYAYGATGHPGIIPPHATLVFDVELLKLESGGGSGVDGFGDVGALESLRGNADLAYILSMEPCGHCLIINNVNFCRESGLRTRTGSNIDCEKLRRRFSSLHFMVEVKGDLTAKKMVLALLELARQDHGALDCCVVVILSHGCQASHLQFPGAVYGTDGCPVSVEKIVNIFNGTSCPSLGGKPKLFFIQACGGEQKDHGFEVASTSPEDESPGSNPEPDATPFQEGLRTFDQLDAISSLPTPSDIFVSYSTFPGFVSWRDPKSGSWYVETLDDIFEQWAHSEDLQSLLLRVANAVSVKGIYKQMPGCFNFLRKKLFFKTSAS

SEQ ID NO: 2 - protein, 18 aa, synthetic construct
EGRGSLLTCGDVEENPGP

SEQ ID NO: 3 - protein, 21 aa, synthetic construct
MEFGLSWLFLVAILKGVQCSR

SEQ ID NO: 4 - protein, 247 aa, synthetic construct
DILLTQTPLSLPVSLGDQASISCRSSQSLVHRNGNTYLHWYLQKPGQSPKLLIHKVSNRFSGVPDRFSGSGSGTDFTLKISRVEAEDLGVYFCSQSTHVPPLTFGAGTKLELKRADAAPTVSIFPGSGGGGSGGEVKLQQSGPSLVEPGASVMISCKASGSSFTGYNMNWVRQNIGKSLEWIGAIDPYYGGTSYNQKFKGRATLTVDKSSSTAYMHLKSLTSEDSAVYYCVSGMEYWGQGTSVTVSS

SEQ ID NO: 5 - protein, 11 aa, synthetic construct
QSLVHRNGNTY

SEQ ID NO: 6 - protein, 23 aa, synthetic construct
CDIYIWAPLAGTCGVLLLSLVIT

SEQ ID NO: 7 - protein, 7 aa, synthetic construct
SQSTHVP

SEQ ID NO: 8 - protein, 8 aa, synthetic construct
GSSFTGYN

SEQ ID NO: 9 - protein, 8 aa, synthetic construct
IDPYYGGT

SEQ ID NO: 10 - protein, 6 aa, synthetic construct
VSGMEY

SEQ ID NO: 11 - protein, 125 aa, synthetic construct
DILLTQTPLSLPVSLGDQASISCRSSQSLVHRNGNTYLHWYLQKPGQSPKLLIHKVSNRFSGVPDRFSGSGSGTDFTLKISRVEAEDLGVYFCSQSTHVPPLTFGAGTKLELKRADAAPTVSIFP

SEQ ID NO: 12 - protein, 113 aa, synthetic construct
EVKLQQSGPSLVEPGASVMISCKASGSSFTGYNMNWVRQNIGKSLEWIGAIDPYYGGTSYNQKFKGRATLTVDKSSSTAYMHLKSLTSEDSAVYYCVSGMEYWGQGTSVTVSS

SEQ ID NO: 13 - protein, 9 aa, synthetic construct
GSGGGGSGG

SEQ ID NO: 14 - protein, 9 aa, synthetic construct
GGGGSGGGG

SEQ ID NO: 15 - protein, 7 aa, synthetic construct
GGGGSGG

SEQ ID NO: 16 - protein, 8 aa, synthetic construct
GGGSGGGG

SEQ ID NO: 17 - protein, 9 aa, synthetic construct
SGGGGSGGG

SEQ ID NO: 18 - protein, 11 aa, synthetic construct
SGGGGSGGGGS

SEQ ID NO: 19 - protein, 12 aa, synthetic construct
SGGGGSGGGGSG

SEQ ID NO: 20 - protein, 14 aa, synthetic construct
SGGGGSGGGGSGGG

SEQ ID NO: 21 - protein, 10 aa, synthetic construct
SGGGGSGGGG

SEQ ID NO: 22 - protein, 13 aa, synthetic construct
SGGGGSGGGGSGG

SEQ ID NO: 23 - protein, 32 aa, synthetic construct
AKTTPPSVYGRVTVSSAEPKSCDKTHTCPPCP

SEQ ID NO: 24 - protein, 40 aa, synthetic construct
PAPRPPTPAPTIASQPLSLRPEACRPAAGGAVHTRGLDFA

SEQ ID NO: 25 - protein, 45 aa, synthetic construct
TTTPAPRPPTPAPTIASQPLSLRPEACRPAAGGAVHTRGLDFACD

SEQ ID NO: 26 - protein, 38 aa, synthetic construct
EVMYPPPYLDNEKSNGTIIHVKGKHLCPSPLFPGPSKP

SEQ ID NO: 27 - protein, 229 aa, synthetic construct
ESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 28 - protein, 119 aa, synthetic construct
ESKYGPPCPSCPGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 29 - protein, 27 aa, synthetic construct
FWVLVVVGGVLACYSLLVTVAFIIFWV

SEQ ID NO: 30 - protein, 41 aa, synthetic construct
RSKRSRLLHSDYMNMTPRRPGPTRKHYQPYAPPRDFAAYRS

SEQ ID NO: 31 - protein, 42 aa, synthetic construct
KRGRKKLLYIFKQPFMRPVQTTQEEDGCSCRFPEEEEGGCEL

SEQ ID NO: 32 - protein, 36 aa, synthetic construct
RDQRLPPDAHKPPGGGSFRTPIQEEQADAHSTLAKI

SEQ ID NO: 33 - DNA, 1206 bp, synthetic construct
  source 1..1206: mol_type = other DNA
atgctcgagggagtgcaggtggagactatctccccaggagacgggcgcaccttccccaagcgcggccagacctgcgtggtgcactacaccgggatgcttgaagatggaaagaaagttgattcctcccgggacagaaacaagccctttaagtttatgctaggcaagcaggaggtgatccgaggctgggaagaaggggttgcccagatgagtgtgggtcagagagccaaactgactatatctccagattatgcctatggtgccactgggcacccaggcatcatcccaccacatgccactctcgtcttcgatgtggagcttctaaaactggaatctggcggtggatccggagtcgacggatttggtgatgtcggtgctcttgagagtttgaggggaaatgcagatttggcttacatcctgagcatggagccctgtggccactgcctcattatcaacaatgtgaacttctgccgtgagtccgggctccgcacccgcactggctccaacatcgactgtgagaagttgcggcgtcgcttctcctcgctgcatttcatggtggaggtgaagggcgacctgactgccaagaaaatggtgctggctttgctggagctggcgcggcaggaccacggtgctctggactgctgcgtggtggtcattctctctcacggctgtcaggccagccacctgcagttcccaggggctgtctacggcacagatggatgccctgtgtcggtcgagaagattgtgaacatcttcaatgggaccagctgccccagcctgggagggaagcccaagctctttttcatccaggcctgtggtggggagcagaaagaccatgggtttgaggtggcctccacttcccctgaagacgagtcccctggcagtaaccccgagccagatgccaccccgttccaggaaggtttgaggaccttcgaccagctggacgccatatctagtttgcccacacccagtgacatctttgtgtcctactctactttcccaggttttgtttcctggagggaccccaagagtggctcctggtacgttgagaccctggacgacatctttgagcagtgggctcactctgaagacctgcagtccctcctgcttagggtcgctaatgctgtttcggtgaaagggatttataaacagatgcctggttgctttaatttcctccggaaaaaacttttctttaaaacatcagctagc

SEQ ID NO: 34 - DNA, 54 bp, synthetic construct
  source 1..54: mol_type = other DNA
gagggcaggggaagtcttctaacatgcggggacgtggaggaaaatcccgggccc

SEQ ID NO: 35 - DNA, 63 bp, synthetic construct
  source 1..63: mol_type = other DNA
atggagtttgggctgagctggctttttcttgtggctattttaaaaggtgtccagtgctctaga

SEQ ID NO: 36 - DNA, 375 bp, synthetic construct
  source 1..375: mol_type = other DNA
gatattttgctgacccaaactccactctccctgcctgtcagtcttggagatcaagcctccatctcttgcagatctagtcagagtcttgtacaccgtaatggaaacacctatttacattggtacctgcagaagccaggccagtctccaaagctcctgattcacaaagtttccaaccgattttctggggtcccagacaggttcagtggcagtggatcagggacagatttcacactcaagatcagcagagtggaggctgaggatctgggagtttatttctgttctcaaagtacacatgttcctccgctcacgttcggtgctgggaccaagctggagctgaaacgggctgatgctgcaccaactgtatccatcttccca

SEQ ID NO: 37 - DNA, 339 bp, synthetic construct
  source 1..339: mol_type = other DNA
gaggtgaagcttcagcagtctggacctagcctggtggagcctggcgcttcagtgatgatatcctgcaaggcttctggttcctcattcactggctacaacatgaactgggtgaggcagaacattggaaagagccttgaatggattggagctattgatccttactatggtggaactagctacaaccagaagttcaagggcagggccacattgactgtagacaaatcgtccagcacagcctacatgcacctcaagagcctgacatctgaggactctgcagtctattactgtgtaagcggaatggagtactggggtcaaggaacctcagtcaccgtctcctca

SEQ ID NO: 38 - DNA, 33 bp, synthetic construct
  source 1..33: mol_type = other DNA
cagagtcttgtacaccgtaatggaaacacctat

SEQ ID NO: 39 - protein, 525 aa, synthetic construct
MEFGLSWLFLVAILKGVQCSRDILLTQTPLSLPVSLGDQASISCRSSQSLVHRNGNTYLHWYLQKPGQSPKLLIHKVSNRFSGVPDRFSGSGSGTDFTLKISRVEAEDLGVYFCSQSTHVPPLTFGAGTKLELKRADAAPTVSIFPGSGGGGSGGEVKLQQSGPSLVEPGASVMISCKASGSSFTGYNMNWVRQNIGKSLEWIGAIDPYYGGTSYNQKFKGRATLTVDKSSSTAYMHLKSLTSEDSAVYYCVSGMEYWGQGTSVTVSSAKTTPPSVYGRVTVSSAEPKSCDKTHTCPPCPDPKFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 40 - DNA, 23 bp, synthetic construct
  source 1..23: mol_type = other DNA
tctcaaagtacacatgttcctcc

SEQ ID NO: 41 - DNA, 24 bp, synthetic construct
  source 1..24: mol_type = other DNA
ggttcctcattcactggctacaac

SEQ ID NO: 42 - DNA, 24 bp, synthetic construct
  source 1..24: mol_type = other DNA
attgatccttactatggtggaact

SEQ ID NO: 43 - DNA, 18 bp, synthetic construct
  source 1..18: mol_type = other DNA
gtaagcggaatggagtac

SEQ ID NO: 44 - DNA, 27 bp, synthetic construct
  source 1..27: mol_type = other DNA
ggctcgggcggtggtgggtcgggtggc

SEQ ID NO: 45 - DNA, 96 bp, synthetic construct
  source 1..96: mol_type = other DNA
gccaaaacgacacccccatcagtctatggaagggtcaccgtctcttcagcggagcccaaatcttgtgacaaaactcacacatgcccaccgtgcccg

SEQ ID NO: 46 - DNA, 81 bp, synthetic construct
  source 1..81: mol_type = other DNA
ttttgggtgctggtggtggttggtggagtcctggcttgctatagcttgctagtaacagtggcctttattattttctgggtg

SEQ ID NO: 47 - DNA, 123 bp, synthetic construct
  source 1..123: mol_type = other DNA
aggagtaagaggagcaggctcctgcacagtgactacatgaacatgactccccgccgccccgggcccacccgcaagcattaccagccctatgccccaccacgcgacttcgcagcctatcgctcc

SEQ ID NO: 48 - DNA, 126 bp, synthetic construct
  source 1..126: mol_type = other DNA
aaacggggcagaaagaaactcctgtatatattcaaacaaccatttatgagaccagtacaaactactcaagaggaagatggctgtagctgccgatttccagaagaagaagaaggaggatgtgaactg

SEQ ID NO: 49 - DNA, 339 bp, synthetic construct
  source 1..339: mol_type = other DNA
agagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 50 - DNA, 2844 bp, synthetic construct
  source 1..2844: mol_type = other DNA
atgctcgagggagtgcaggtggagactatctccccaggagacgggcgcaccttccccaagcgcggccagacctgcgtggtgcactacaccgggatgcttgaagatggaaagaaagttgattcctcccgggacagaaacaagccctttaagtttatgctaggcaagcaggaggtgatccgaggctgggaagaaggggttgcccagatgagtgtgggtcagagagccaaactgactatatctccagattatgcctatggtgccactgggcacccaggcatcatcccaccacatgccactctcgtcttcgatgtggagcttctaaaactggaatctggcggtggatccggagtcgacggatttggtgatgtcggtgctcttgagagtttgaggggaaatgcagatttggcttacatcctgagcatggagccctgtggccactgcctcattatcaacaatgtgaacttctgccgtgagtccgggctccgcacccgcactggctccaacatcgactgtgagaagttgcggcgtcgcttctcctcgctgcatttcatggtggaggtgaagggcgacctgactgccaagaaaatggtgctggctttgctggagctggcgcggcaggaccacggtgctctggactgctgcgtggtggtcattctctctcacggctgtcaggccagccacctgcagttcccaggggctgtctacggcacagatggatgccctgtgtcggtcgagaagattgtgaacatcttcaatgggaccagctgccccagcctgggagggaagcccaagctctttttcatccaggcctgtggtggggagcagaaagaccatgggtttgaggtggcctccacttcccctgaagacgagtcccctggcagtaaccccgagccagatgccaccccgttccaggaaggtttgaggaccttcgaccagctggacgccatatctagtttgcccacacccagtgacatctttgtgtcctactctactttcccaggttttgtttcctggagggaccccaagagtggctcctggtacgttgagaccctggacgacatctttgagcagtgggctcactctgaagacctgcagtccctcctgcttagggtcgctaatgctgtttcggtgaaagggatttataaacagatgcctggttgctttaatttcctccggaaaaaacttttctttaaaacatcagctagcagagccgagggcaggggaagtcttctaacatgcggggacgtggaggaaaatcccgggcccatggagtttgggctgagctggctttttcttgtggctattttaaaaggtgtccagtgctctagagatattttgctgacccaaactccactctccctgcctgtcagtcttggagatcaagcctccatctcttgcagatctagtcagagtcttgtacaccgtaatggaaacacctatttacattggtacctgcagaagccaggccagtctccaaagctcctgattcacaaagtttccaaccgattttctggggtcccagacaggttcagtggcagtggatcagggacagatttcacactcaagatcagcagagtggaggctgaggatctgggagtttatttctgttctcaaagtacacatgttcctccgctcacgttcggtgctgggaccaagctggagctgaaacgggctgatgctgcaccaactgtatccatcttcccaggctcgggcggtggtgggtcgggtggcgaggtgaagcttcagcagtctggacctagcctggtggagcctggcgcttcagtgatgatatcctgcaaggcttctggttcctcattcactggctacaacatgaactgggtgaggcagaacattggaaagagccttgaatggattggagctattgatccttactatggtggaactagctacaaccagaagttcaagggcagggccacattgactgtagacaaatcgtccagcacagcctacatgcacctcaagagcctgacatctgaggactctgcagtctattactgtgtaagcggaatggagtactggggtcaaggaacctcagtcaccgtctcctcagccaaaacgacacccccatcagtctatggaagggtcaccgtctcttcagcggagcccaaatcttgtgacaaaactcacacatgcccaccgtgcccggatcccaaattttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 51 - protein, 7 aa, synthetic construct
LYCNHRN

SEQ ID NO: 52 - DNA, 1578 bp, synthetic construct
  source 1..1578: mol_type = other DNA
atggagtttgggctgagctggctttttcttgtggctattttaaaaggtgtccagtgctctagagatattttgctgacccaaactccactctccctgcctgtcagtcttggagatcaagcctccatctcttgcagatctagtcagagtcttgtacaccgtaatggaaacacctatttacattggtacctgcagaagccaggccagtctccaaagctcctgattcacaaagtttccaaccgattttctggggtcccagacaggttcagtggcagtggatcagggacagatttcacactcaagatcagcagagtggaggctgaggatctgggagtttatttctgttctcaaagtacacatgttcctccgctcacgttcggtgctgggaccaagctggagctgaaacgggctgatgctgcaccaactgtatccatcttcccaggctcgggcggtggtgggtcgggtggcgaggtgaagcttcagcagtctggacctagcctggtggagcctggcgcttcagtgatgatatcctgcaaggcttctggttcctcattcactggctacaacatgaactgggtgaggcagaacattggaaagagccttgaatggattggagctattgatccttactatggtggaactagctacaaccagaagttcaagggcagggccacattgactgtagacaaatcgtccagcacagcctacatgcacctcaagagcctgacatctgaggactctgcagtctattactgtgtaagcggaatggagtactggggtcaaggaacctcagtcaccgtctcctcagccaaaacgacacccccatcagtctatggaagggtcaccgtctcttcagcggagcccaaatcttgtgacaaaactcacacatgcccaccgtgcccggatcccaaattttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 53 - protein, 112 aa, synthetic construct
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR